SEQUENCE LISTING (198 sequences)

SEQ ID NO: 1 - DNA, 4968 bp, Severe acute respiratory syndrome coronavirus 2
ttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggt

SEQ ID NO: 2 - protein, 1255 aa, Severe acute respiratory syndrome coronavirus 2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 422 aa, Severe acute respiratory syndrome coronavirus 2
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 4 - protein, 221 aa, Severe acute respiratory syndrome coronavirus 2
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 5 - protein, 76 aa, Severe acute respiratory syndrome coronavirus 2
MYSFVSEETGTLIVNSVLLFLAFVVFLLVTLAILTALRLCAYCCNIVNVSLVKPTVYVYSRVKNLNSSEGVPDLLV

SEQ ID NO: 6 - protein, 30 aa, Artificial Sequence
  Description of Artificial Sequence Synthetic polypeptide
FKNIDGYFKIYSKHTPINLVRDLPQGFSAL

SEQ ID NO: 7 - protein, 30 aa, Artificial Sequence
  Description of Artificial Sequence Synthetic polypeptide
PTKLNDLCFTNVYADSFVIRGDEVRQIAPG

SEQ ID NO: 8 - protein, 30 aa, Artificial Sequence
  Description of Artificial Sequence Synthetic polypeptide
IRGDEVRQIAPGQTGKIADYNYKLPDDFTG

SEQ ID NO: 9 - protein, 30 aa, Artificial Sequence
  Description of Artificial Sequence Synthetic polypeptide
RLFRKSNLKPFERDISTEIYQAGSTPCNGC

SEQ ID NO: 10 - protein, 30 aa, Artificial Sequence
  Description of Artificial Sequence Synthetic polypeptide
GVEGFNCYFPLQSYGFQPTNGVGYQPYRVV

SEQ ID NO: 11 - protein, 15 aa, Homo sapiens
MWFLTTLLLWVPVDG

SEQ ID NO: 12 - protein, 28 aa, Homo sapiens
MAAPGSARRPLLLLLLLLLLGLMHCASA

SEQ ID NO: 13 - protein, 28 aa, Homo sapiens
MVCFRLFPVPGSGLVLVCLVLGAVRSYA

SEQ ID NO: 14 - protein, 24 aa, Homo sapiens
MVVMAPRTLFLLLSGALTLTETWA

SEQ ID NO: 15 - protein, 25 aa, Homo sapiens
MAISGVPVLGFFIIAVLMSAQESWA

SEQ ID NO: 16 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 17 - protein, 6 aa, Artificial Sequence
  Description of Artificial Sequence Synthetic peptide
CKAAKN

SEQ ID NO: 18 - protein, 5 aa, Artificial Sequence
  Description of Artificial Sequence Synthetic peptide
CREKA

SEQ ID NO: 19 - protein, 7 aa, Artificial Sequence
  Description of Artificial Sequence Synthetic peptide
ARRPKLD

SEQ ID NO: 20 - protein, 8 aa, Artificial Sequence
  Description of Artificial Sequence Synthetic peptide
DYKDDDDK

SEQ ID NO: 21 - protein, 15 aa, Artificial Sequence
  Description of Artificial Sequence Synthetic peptide
  SITE (1)..(15): This sequence may encompass 1-3 “Glu Ala Ala Ala Lys” repeating units
EAAAKEAAAKEAAAK

SEQ ID NO: 22 - protein, 46 aa, Artificial Sequence
  Description of Artificial Sequence Synthetic polypeptide
AEAAAKEAAAKEAAAKEAAAKALEAEAAAKEAAAKEAAAKEAAAKA

SEQ ID NO: 23 - protein, 5 aa, Artificial Sequence
  Description of Artificial Sequence Synthetic peptide
PAPAP

SEQ ID NO: 24 - protein, 12 aa, Artificial Sequence
  Description of Artificial Sequence Synthetic peptide
AEAAAKEAAAKA

SEQ ID NO: 25 - protein, 68 aa, Artificial Sequence
  Description of Artificial Sequence Synthetic polypeptide
  SITE (1)..(68): This sequence may encompass 10-34 “Ala Pro” repeating units
APAPAPAPAPAPAPAPAPAPAPAPAPAPAPAPAPAPAPAPAPAPAPAPAPAPAPAPAPAPAPAPAPAP

SEQ ID NO: 26 - protein, 20 aa, Artificial Sequence
  Description of Artificial Sequence Synthetic peptide
  SITE (1)..(20): This sequence may encompass 1-4 “Gly Gly Gly Gly Ser” repeating units
GGGGSGGGGSGGGGSGGGGS

SEQ ID NO: 27 - protein, 8 aa, Artificial Sequence
  Description of Artificial Sequence Synthetic peptide
  SITE (1)..(8): This sequence may encompass 6-8 residues
GGGGGGGG

SEQ ID NO: 28 - protein, 17 aa, Artificial Sequence
  Description of Artificial Sequence Synthetic peptide
VSQTSKLTRAETVFPDV

SEQ ID NO: 29 - protein, 6 aa, Artificial Sequence
  Description of Artificial Sequence Synthetic peptide
PLGLWA

SEQ ID NO: 30 - protein, 6 aa, Artificial Sequence
  Description of Artificial Sequence Synthetic peptide
RVLAEA

SEQ ID NO: 31 - protein, 10 aa, Artificial Sequence
  Description of Artificial Sequence Synthetic peptide
EDVVCCSMSY

SEQ ID NO: 32 - protein, 8 aa, Artificial Sequence
  Description of Artificial Sequence Synthetic peptide
GGIEGRGS

SEQ ID NO: 33 - protein, 10 aa, Artificial Sequence
  Description of Artificial Sequence Synthetic peptide
TRHRQPRGWE

SEQ ID NO: 34 - protein, 10 aa, Artificial Sequence
  Description of Artificial Sequence Synthetic peptide
AGNRVRRSVG

SEQ ID NO: 35 - protein, 9 aa, Artificial Sequence
  Description of Artificial Sequence Synthetic peptide
RRRRRRRRR

SEQ ID NO: 36 - protein, 4 aa, Artificial Sequence
  Description of Artificial Sequence Synthetic peptide
GFLG

SEQ ID NO: 37 - protein, 21 aa, Artificial Sequence
  Description of Artificial Sequence Synthetic peptide
YKDHDGDYKDHDIDYKDDDDK

SEQ ID NO: 38 - protein, 16 aa, Artificial Sequence
  Description of Artificial Sequence Synthetic peptide
FKESWREARGTRIERG

SEQ ID NO: 39 - protein, 12 aa, Artificial Sequence
  Description of Artificial Sequence Synthetic peptide
SVSVGMKPSPRP

SEQ ID NO: 40 - protein, 12 aa, Artificial Sequence
  Description of Artificial Sequence Synthetic peptide
PRPSPKMGVSVS

SEQ ID NO: 41 - protein, 12 aa, Artificial Sequence
  Description of Artificial Sequence Synthetic peptide
TDSILRSYDWTY

SEQ ID NO: 42 - protein, 9 aa, Artificial Sequence
  Description of Artificial Sequence Synthetic peptide
CSNIDARAC

SEQ ID NO: 43 - protein, 9 aa, Artificial Sequence
  Description of Artificial Sequence Synthetic peptide
CGNSNPKSC

SEQ ID NO: 44 - protein, 12 aa, Artificial Sequence
  Description of Artificial Sequence Synthetic peptide
GRRTRSRRLRRS

SEQ ID NO: 45 - protein, 9 aa, Artificial Sequence
  Description of Artificial Sequence Synthetic peptide
CTKNSYLMC

SEQ ID NO: 46 - protein, 12 aa, Artificial Sequence
  Description of Artificial Sequence Synthetic peptide
AADNAKTKSFPV

SEQ ID NO: 47 - protein, 7 aa, Artificial Sequence
  Description of Artificial Sequence Synthetic peptide
CRGRRST

SEQ ID NO: 48 - protein, 6 aa, Artificial Sequence
  Description of Artificial Sequence Synthetic peptide
CRSRKG

SEQ ID NO: 49 - protein, 12 aa, Artificial Sequence
  Description of Artificial Sequence Synthetic peptide
FRPNRAQDYNTN

SEQ ID NO: 50 - protein, 15 aa, Artificial Sequence
  Description of Artificial Sequence Synthetic peptide
IAGLATPGWSHWLAL

SEQ ID NO: 51 - protein, 9 aa, Artificial Sequence
  Description of Artificial Sequence Synthetic peptide
CREAGRKAC

SEQ ID NO: 52 - protein, 9 aa, Artificial Sequence
  Description of Artificial Sequence Synthetic peptide
CAGRRSAYC

SEQ ID NO: 53 - protein, 9 aa, Artificial Sequence
  Description of Artificial Sequence Synthetic peptide
CSRPRRSEC

SEQ ID NO: 54 - protein, 5 aa, Artificial Sequence
  Description of Artificial Sequence Synthetic peptide
CGKRK

SEQ ID NO: 55 - protein, 5 aa, Artificial Sequence
  Description of Artificial Sequence Synthetic peptide
CDTRL

SEQ ID NO: 56 - protein, 10 aa, Artificial Sequence
  Description of Artificial Sequence Synthetic peptide
TAASGVRSMH

SEQ ID NO: 57 - protein, 10 aa, Artificial Sequence
  Description of Artificial Sequence Synthetic peptide
LTLRWVGLMS

SEQ ID NO: 58 - protein, 9 aa, Artificial Sequence
  Description of Artificial Sequence Synthetic peptide
CVNHPAFAC

SEQ ID NO: 59 - protein, 9 aa, Artificial Sequence
  Description of Artificial Sequence Synthetic peptide
CLSDGKRKC

SEQ ID NO: 60 - protein, 12 aa, Artificial Sequence
  Description of Artificial Sequence Synthetic peptide
KSLSRHDHIHHH

SEQ ID NO: 61 - protein, 12 aa, Artificial Sequence
  Description of Artificial Sequence Synthetic peptide
SFSIIHTPILPL

SEQ ID NO: 62 - protein, 9 aa, Artificial Sequence
  Description of Artificial Sequence Synthetic peptide
CPHSKPCLC

SEQ ID NO: 63 - protein, 9 aa, Artificial Sequence
  Description of Artificial Sequence Synthetic peptide
CPIEDRPMC

SEQ ID NO: 64 - protein, 9 aa, Artificial Sequence
  Description of Artificial Sequence Synthetic peptide
CTPSPFSHC

SEQ ID NO: 65 - protein, 7 aa, Artificial Sequence
  Description of Artificial Sequence Synthetic peptide
VHLGYAT

SEQ ID NO: 66 - protein, 9 aa, Artificial Sequence
  Description of Artificial Sequence Synthetic peptide
CSNRDARRC

SEQ ID NO: 67 - protein, 9 aa, Artificial Sequence
  Description of Artificial Sequence Synthetic peptide
CQDGRMGFC

SEQ ID NO: 68 - protein, 12 aa, Artificial Sequence
  Description of Artificial Sequence Synthetic peptide
LWATFPPRPPWL

SEQ ID NO: 69 - protein, 12 aa, Artificial Sequence
  Description of Artificial Sequence Synthetic peptide
LLADTTHHRPWT

SEQ ID NO: 70 - protein, 8 aa, Artificial Sequence
  Description of Artificial Sequence Synthetic peptide
CDGLGDDC

SEQ ID NO: 71 - protein, 8 aa, Artificial Sequence
  Description of Artificial Sequence Synthetic peptide
CDGWGPNC

SEQ ID NO: 72 - protein, 12 aa, Artificial Sequence
  Description of Artificial Sequence Synthetic peptide
RLLDTNRPLLPY

SEQ ID NO: 73 - protein, 12 aa, Artificial Sequence
  Description of Artificial Sequence Synthetic peptide
TSPLNIHNGQKL

SEQ ID NO: 74 - protein, 13 aa, Artificial Sequence
  Description of Artificial Sequence Synthetic peptide
SPRGDLAVLGHKY

SEQ ID NO: 75 - protein, 7 aa, Artificial Sequence
  Description of Artificial Sequence Synthetic peptide
SIGYPLP

SEQ ID NO: 76 - protein, 7 aa, Artificial Sequence
  Description of Artificial Sequence Synthetic peptide
LSIPPKA

SEQ ID NO: 77 - protein, 7 aa, Artificial Sequence
  Description of Artificial Sequence Synthetic peptide
FQTPPQL

SEQ ID NO: 78 - protein, 7 aa, Artificial Sequence
  Description of Artificial Sequence Synthetic peptide
LTPATAI

SEQ ID NO: 79 - protein, 17 aa, Artificial Sequence
  Description of Artificial Sequence Synthetic peptide
CNIWGVVLSWIGVFPEC

SEQ ID NO: 80 - protein, 5 aa, Artificial Sequence
  Description of Artificial Sequence Synthetic peptide
NTTTH

SEQ ID NO: 81 - protein, 7 aa, Artificial Sequence
  Description of Artificial Sequence Synthetic peptide
VHPKQHR

SEQ ID NO: 82 - protein, 18 aa, Artificial Sequence
  Description of Artificial Sequence Synthetic peptide
CRKRLDRNCCRTLTVRKC

SEQ ID NO: 83 - protein, 9 aa, Artificial Sequence
  Description of Artificial Sequence Synthetic peptide
CLWTVGGGC

SEQ ID NO: 84 - protein, 12 aa, Artificial Sequence
  Description of Artificial Sequence Synthetic peptide
QPWLEQAYYSTF

SEQ ID NO: 85 - protein, 12 aa, Artificial Sequence
  Description of Artificial Sequence Synthetic peptide
YPHIDSLGHWRR

SEQ ID NO: 86 - protein, 12 aa, Artificial Sequence
  Description of Artificial Sequence Synthetic peptide
SAHGTSTGVPWP

SEQ ID NO: 87 - protein, 12 aa, Artificial Sequence
  Description of Artificial Sequence Synthetic peptide
VPWMEPAYQRFL

SEQ ID NO: 88 - protein, 12 aa, Artificial Sequence
  Description of Artificial Sequence Synthetic peptide
TLPWLEESYWRP

SEQ ID NO: 89 - protein, 4 aa, Artificial Sequence
  Description of Artificial Sequence Synthetic peptide
HWRR

SEQ ID NO: 90 - protein, 9 aa, Artificial Sequence
  Description of Artificial Sequence Synthetic peptide
CSTSMLKAC

SEQ ID NO: 91 - protein, 7 aa, Artificial Sequence
  Description of Artificial Sequence Synthetic peptide
DDTRHWG

SEQ ID NO: 92 - protein, 6 aa, Artificial Sequence
  Description of Artificial Sequence Synthetic peptide
CARPAR

SEQ ID NO: 93 - protein, 6 aa, Artificial Sequence
  Description of Artificial Sequence Synthetic peptide
CKRAVR

SEQ ID NO: 94 - protein, 9 aa, Artificial Sequence
  Description of Artificial Sequence Synthetic peptide
CRSTRANPC

SEQ ID NO: 95 - protein, 9 aa, Artificial Sequence
  Description of Artificial Sequence Synthetic peptide
CPKTRRVPC

SEQ ID NO: 96 - protein, 9 aa, Artificial Sequence
  Description of Artificial Sequence Synthetic peptide
CSGMARTKC

SEQ ID NO: 97 - protein, 5 aa, Artificial Sequence
  Description of Artificial Sequence Synthetic peptide
CRPPR

SEQ ID NO: 98 - protein, 9 aa, Artificial Sequence
  Description of Artificial Sequence Synthetic peptide
CRVASVLPC

SEQ ID NO: 99 - protein, 9 aa, Artificial Sequence
  Description of Artificial Sequence Synthetic peptide
SWCEPGWCR

SEQ ID NO: 100 - protein, 20 aa, Artificial Sequence
  Description of Artificial Sequence Synthetic peptide
LSGTPERSGQAVKVKLKAIP

SEQ ID NO: 101 - protein, 18 aa, Artificial Sequence
  Description of Artificial Sequence Synthetic peptide
CHVLWSTRCCVSNPRWKC

SEQ ID NO: 102 - protein, 7 aa, Artificial Sequence
  Description of Artificial Sequence Synthetic peptide
LSALPRT

SEQ ID NO: 103 - protein, 7 aa, Artificial Sequence
  Description of Artificial Sequence Synthetic peptide
CLPVASC

SEQ ID NO: 104 - protein, 9 aa, Artificial Sequence
  Description of Artificial Sequence Synthetic peptide
  MOD_RES (6)..(6): R or M
  MOD_RES (8)..(8): Any amino acid
  MOD_RES (9)..(9): W or L
ELRGDXAXX

SEQ ID NO: 105 - protein, 12 aa, Artificial Sequence
  Description of Artificial Sequence Synthetic peptide
  MOD_RES (3)..(3): K or R
  MOD_RES (5)..(7): Any amino acid
  MOD_RES (8)..(8): T or S
  MOD_RES (11)..(11): Any amino acid
GVXGXXXXRDXR

SEQ ID NO: 106 - protein, 14 aa, Artificial Sequence
  Description of Artificial Sequence Synthetic peptide
HITSLLSHTTHREP

SEQ ID NO: 107 - protein, 16 aa, Artificial Sequence
  Description of Artificial Sequence Synthetic peptide
ANTPCGPYTHDCPVKR

SEQ ID NO: 108 - protein, 21 aa, Artificial Sequence
  Description of Artificial Sequence Synthetic peptide
CGFELETCCGFECVRQCPERC

SEQ ID NO: 109 - protein, 30 aa, Artificial Sequence
  Description of Artificial Sequence Synthetic polypeptide
QPFMQCLCLIYDASCRNVPPIFNDVYWIAF

SEQ ID NO: 110 - protein, 7 aa, Artificial Sequence
  Description of Artificial Sequence Synthetic peptide
VNTANST

SEQ ID NO: 111 - protein, 9 aa, Artificial Sequence
  Description of Artificial Sequence Synthetic peptide
CTSGTHPRC

SEQ ID NO: 112 - protein, 32 aa, Artificial Sequence
  Description of Artificial Sequence Synthetic polypeptide
SGEWVIKEARGWKHWVFYSCCPTTPYLDITYH

SEQ ID NO: 113 - protein, 7 aa, Artificial Sequence
  Description of Artificial Sequence Synthetic peptide
YSGKWGW

SEQ ID NO: 114 - protein, 36 aa, Artificial Sequence
  Description of Artificial Sequence Synthetic polypeptide
LETTCASLCYPSYQCSYTMPHPPVVPPHPMTYSCQY

SEQ ID NO: 115 - protein, 7 aa, Artificial Sequence
  Description of Artificial Sequence Synthetic peptide
YPRLLTP

SEQ ID NO: 116 - protein, 9 aa, Artificial Sequence
  Description of Artificial Sequence Synthetic peptide
CSQSHPRHC

SEQ ID NO: 117 - protein, 9 aa, Artificial Sequence
  Description of Artificial Sequence Synthetic peptide
CSKSSDYQC

SEQ ID NO: 118 - protein, 9 aa, Artificial Sequence
  Description of Artificial Sequence Synthetic peptide
CKSTHPLSC

SEQ ID NO: 119 - protein, 10 aa, Artificial Sequence
  Description of Artificial Sequence Synthetic peptide
CTGKSCLRVG

SEQ ID NO: 120 - protein, 12 aa, Artificial Sequence
  Description of Artificial Sequence Synthetic peptide
SFKPSGLPAQSL

SEQ ID NO: 121 - protein, 9 aa, Artificial Sequence
  Description of Artificial Sequence Synthetic peptide
CTANSSAQC

SEQ ID NO: 122 - protein, 9 aa, Artificial Sequence
  Description of Artificial Sequence Synthetic peptide
CLSSRLDAC

SEQ ID NO: 123 - protein, 9 aa, Artificial Sequence
  Description of Artificial Sequence Synthetic peptide
GHKAKGPRK

SEQ ID NO: 124 - protein, 7 aa, Artificial Sequence
  Description of Artificial Sequence Synthetic peptide
HAIYPRH

SEQ ID NO: 125 - protein, 12 aa, Artificial Sequence
  Description of Artificial Sequence Synthetic peptide
THRPPMWSPVWP

SEQ ID NO: 126 - protein, 13 aa, Artificial Sequence
  Description of Artificial Sequence Synthetic peptide
HLNILSTLWKYRC

SEQ ID NO: 127 - protein, 7 aa, Artificial Sequence
  Description of Artificial Sequence Synthetic peptide
CAGALCY

SEQ ID NO: 128 - protein, 9 aa, Artificial Sequence
  Description of Artificial Sequence Synthetic peptide
CLEVSRKNC

SEQ ID NO: 129 - protein, 12 aa, Artificial Sequence
  Description of Artificial Sequence Synthetic peptide
RPRTRLHTHRNR

SEQ ID NO: 130 - protein, 11 aa, Artificial Sequence
  Description of Artificial Sequence Synthetic peptide
ACTTPHAWLCG

SEQ ID NO: 131 - protein, 14 aa, Artificial Sequence
  Description of Artificial Sequence Synthetic peptide
GLAHSFSDFARDFV

SEQ ID NO: 132 - protein, 15 aa, Artificial Sequence
  Description of Artificial Sequence Synthetic peptide
GYRPVHNIRGHWAPG

SEQ ID NO: 133 - protein, 12 aa, Artificial Sequence
  Description of Artificial Sequence Synthetic peptide
TGNYKALHPHNG

SEQ ID NO: 134 - protein, 9 aa, Artificial Sequence
  Description of Artificial Sequence Synthetic peptide
CRTIGPSVC

SEQ ID NO: 135 - protein, 9 aa, Artificial Sequence
  Description of Artificial Sequence Synthetic peptide
CTSTSAPYC

SEQ ID NO: 136 - protein, 9 aa, Artificial Sequence
  Description of Artificial Sequence Synthetic peptide
CSYTSSTMC

SEQ ID NO: 137 - protein, 8 aa, Artificial Sequence
  Description of Artificial Sequence Synthetic peptide
CMPRLRGC

SEQ ID NO: 138 - protein, 12 aa, Artificial Sequence
  Description of Artificial Sequence Synthetic peptide
TPSYDTYAAELR

SEQ ID NO: 139 - protein, 12 aa, Artificial Sequence
  Description of Artificial Sequence Synthetic peptide
RLSSVDSDLSGC

SEQ ID NO: 140 - protein, 4 aa, Artificial Sequence
  Description of Artificial Sequence Synthetic peptide
CAQK

SEQ ID NO: 141 - protein, 12 aa, Artificial Sequence
  Description of Artificial Sequence Synthetic peptide
SGVYKVAYDWQH

SEQ ID NO: 142 - protein, 7 aa, Artificial Sequence
  Description of Artificial Sequence Synthetic peptide
LMLPRAD

SEQ ID NO: 143 - protein, 9 aa, Artificial Sequence
  Description of Artificial Sequence Synthetic peptide
CSCFRDVCC

SEQ ID NO: 144 - protein, 9 aa, Artificial Sequence
  Description of Artificial Sequence Synthetic peptide
CRDVVSVIC

SEQ ID NO: 145 - protein, 13 aa, Artificial Sequence
  Description of Artificial Sequence Synthetic peptide
CVALCREACGEGC

SEQ ID NO: 146 - protein, 7 aa, Artificial Sequence
  Description of Artificial Sequence Synthetic peptide
GLSGGRS

SEQ ID NO: 147 - protein, 6 aa, Artificial Sequence
  Description of Artificial Sequence Synthetic peptide
WYRGRL

SEQ ID NO: 148 - protein, 9 aa, Artificial Sequence
  Description of Artificial Sequence Synthetic peptide
CPGPEGAGC

SEQ ID NO: 149 - protein, 14 aa, Artificial Sequence
  Description of Artificial Sequence Synthetic peptide
SMSIARLVSFLEYR

SEQ ID NO: 150 - protein, 17 aa, Artificial Sequence
  Description of Artificial Sequence Synthetic peptide
GPEDTSRAPENQQKTGC

SEQ ID NO: 151 - protein, 9 aa, Artificial Sequence
  Description of Artificial Sequence Synthetic peptide
CKGGRAKDC

SEQ ID NO: 152 - protein, 9 aa, Artificial Sequence
  Description of Artificial Sequence Synthetic peptide
CARSKNKDC

SEQ ID NO: 153 - protein, 9 aa, Artificial Sequence
  Description of Artificial Sequence Synthetic peptide
CHAQGSAEC

SEQ ID NO: 154 - protein, 24 aa, Artificial Sequence
  Description of Artificial Sequence Synthetic peptide
LEPRWGFGWWLKLSTHTTESRSMV

SEQ ID NO: 155 - protein, 14 aa, Artificial Sequence
  Description of Artificial Sequence Synthetic peptide
ACSTEALRHCGGGS

SEQ ID NO: 156 - protein, 7 aa, Artificial Sequence
  Description of Artificial Sequence Synthetic peptide
ASSLNIA

SEQ ID NO: 157 - protein, 15 aa, Artificial Sequence
  Description of Artificial Sequence Synthetic peptide
DSLKSYWYLQKFSWR

SEQ ID NO: 158 - protein, 18 aa, Artificial Sequence
  Description of Artificial Sequence Synthetic peptide
DWLKAFYDKVAEKLKEAF

SEQ ID NO: 159 - protein, 18 aa, Artificial Sequence
  Description of Artificial Sequence Synthetic peptide
KSKTEYYNAWAVWERNAP

SEQ ID NO: 160 - protein, 21 aa, Artificial Sequence
  Description of Artificial Sequence Synthetic peptide
GNGEQREMAVSRLRDCLDRQA

SEQ ID NO: 161 - protein, 22 aa, Artificial Sequence
  Description of Artificial Sequence Synthetic peptide
HTPGNSNKWKHLQENKKGRPRR

SEQ ID NO: 162 - protein, 24 aa, Artificial Sequence
  Description of Artificial Sequence Synthetic peptide
RRRRRRRRRGPLGLAGEEEEEEEE

SEQ ID NO: 163 - protein, 23 aa, Artificial Sequence
  Description of Artificial Sequence Synthetic peptide
GAFSWGSLWSGIKNFGSTVKNYG

SEQ ID NO: 164 - protein, 5 aa, Artificial Sequence
  Description of Artificial Sequence Synthetic peptide
RLRWR

SEQ ID NO: 165 - protein, 13 aa, Artificial Sequence
  Description of Artificial Sequence Synthetic peptide
LGQQQPFPPQQPY

SEQ ID NO: 166 - protein, 16 aa, Artificial Sequence
  Description of Artificial Sequence Synthetic peptide
ILGKLLSTAAGLLSNL

SEQ ID NO: 167 - protein, 19 aa, Artificial Sequence
  Description of Artificial Sequence Synthetic peptide
TFFYGGSRGKRNNFKTEEY

SEQ ID NO: 168 - protein, 12 aa, Artificial Sequence
  Description of Artificial Sequence Synthetic peptide
  MOD_RES (11)..(11): Any amino acid
  MOD_RES (12)..(12): Bpg-Gly
LRKLRKRLLRXG

SEQ ID NO: 169 - protein, 10 aa, Artificial Sequence
  Description of Artificial Sequence Synthetic peptide
LRKLRKRLLR

SEQ ID NO: 170 - protein, 22 aa, Artificial Sequence
  Description of Artificial Sequence Synthetic peptide
MVRRFLVTLRIRRACGPPRVRV

SEQ ID NO: 171 - protein, 10 aa, Artificial Sequence
  Description of Artificial Sequence Synthetic peptide
TWKTSRISIF

SEQ ID NO: 172 - protein, 10 aa, Artificial Sequence
  Description of Artificial Sequence Synthetic peptide
FGRLVSSIRY

SEQ ID NO: 173 - protein, 18 aa, Artificial Sequence
  Description of Artificial Sequence Synthetic peptide
EPIHRSTLTALLHWRGWV

SEQ ID NO: 174 - protein, 6 aa, Artificial Sequence
  Description of Artificial Sequence Synthetic peptide
HYFKFD

SEQ ID NO: 175 - protein, 6 aa, Artificial Sequence
  Description of Artificial Sequence Synthetic peptide
HFRRHL

SEQ ID NO: 176 - protein, 6 aa, Artificial Sequence
  Description of Artificial Sequence Synthetic peptide
  MOD_RES (3)..(3): Cit
HWXGWV

SEQ ID NO: 177 - protein, 5 aa, Artificial Sequence
  Description of Artificial Sequence Synthetic peptide
DDAAG

SEQ ID NO: 178 - protein, 4 aa, Artificial Sequence
  Description of Artificial Sequence Synthetic peptide
DAAG

SEQ ID NO: 179 - protein, 9 aa, Artificial Sequence
  Description of Artificial Sequence Synthetic peptide
  MOD_RES (9)..(9): Lact-E
SARWHYFKE

SEQ ID NO: 180 - protein, 8 aa, Artificial Sequence
  Description of Artificial Sequence Synthetic peptide
  MOD_RES (1)..(1): Dap(A)
  MOD_RES (8)..(8): Lact-E
ARWHYFKE

SEQ ID NO: 181 - protein, 7 aa, Artificial Sequence
  Description of Artificial Sequence Synthetic peptide
MWFRHYK

SEQ ID NO: 182 - protein, 8 aa, Artificial Sequence
  Description of Artificial Sequence Synthetic peptide
NKFRGKYK

SEQ ID NO: 183 - protein, 8 aa, Artificial Sequence
  Description of Artificial Sequence Synthetic peptide
NARKFYKG

SEQ ID NO: 184 - protein, 8 aa, Artificial Sequence
  Description of Artificial Sequence Synthetic peptide
FYWHCLDE

SEQ ID NO: 185 - protein, 8 aa, Artificial Sequence
  Description of Artificial Sequence Synthetic peptide
FYCHWALE

SEQ ID NO: 186 - protein, 8 aa, Artificial Sequence
  Description of Artificial Sequence Synthetic peptide
FYCHTIDE

SEQ ID NO: 187 - protein, 4 aa, Artificial Sequence
  Description of Artificial Sequence Synthetic peptide
RRGW

SEQ ID NO: 188 - protein, 7 aa, Artificial Sequence
  Description of Artificial Sequence Synthetic peptide
KHRFNKD

SEQ ID NO: 189 - protein, 4 aa, Artificial Sequence
  Description of Artificial Sequence Synthetic peptide
APAR

SEQ ID NO: 190 - protein, 7 aa, Artificial Sequence
  Description of Artificial Sequence Synthetic peptide
CKAAKNK

SEQ ID NO: 191 - protein, 14 aa, Artificial Sequence
  Description of Artificial Sequence Synthetic peptide
CKAAKNDYKDDDDK

SEQ ID NO: 192 - protein, 13 aa, Artificial Sequence
  Description of Artificial Sequence Synthetic peptide
CREKADYKDDDDK

SEQ ID NO: 193 - protein, 15 aa, Artificial Sequence
  Description of Artificial Sequence Synthetic peptide
ARRPKLDDYKDDDDK

SEQ ID NO: 194 - DNA, 24 bp, Artificial Sequence
  Description of Artificial Sequence Synthetic oligonucleotide
gattataaagatgatgatgataaa

SEQ ID NO: 195 - DNA, 42 bp, Artificial Sequence
  Description of Artificial Sequence Synthetic oligonucleotide
tgcaaagcggcgaaaaacgattataaagatgatgatgataaa

SEQ ID NO: 196 - DNA, 39 bp, Artificial Sequence
  Description of Artificial Sequence Synthetic oligonucleotide
tgccgcgaaaaagcggattataaagatgatgatgataaa

SEQ ID NO: 197 - DNA, 45 bp, Artificial Sequence
  Description of Artificial Sequence Synthetic oligonucleotide
gcgcgccgcccgaaactggatgattataaagatgatgatgataaa

SEQ ID NO: 198 - protein, 30 aa, Artificial Sequence
  Description of Artificial Sequence Synthetic polypeptide
RLFRKSNLKPFERDISTEIYQAGSTPCNGV

(19) United States
(12) Patent Application Publication
(10) Pub. No.: US 2024/0299529 A1
(43) Pub. Date: Sep. 12, 2024

(54) ADAPTER POLYPEPTIDES AND METHODS OF USING THE SAME

(71) Applicants: OHIO STATE INNOVATION FOUNDATION, Columbus, OH (US); SPOT BIOSYSTEMS LTD., Palo Alto, CA (US)

(72) Inventors: Ly James LEE, Columbus, OH (US); Chi-Ling CHIANG, Columbus, OH (US); Yifan MA, Columbus, OH (US)

(21) Appl. No.: 18/018,519

(22) PCT Filed: Aug. 4, 2021
(86) PCT No.: PCT/US2021/044449

§ 371 Date: Jan. 27, 2023

Related U.S. Application Data
(60) Provisional application No. 63/061,749, filed on Aug. 5, 2020.

(51) Int. Cl. A61K 39/215; A61K 39/00; A61K 47/69; C07K 14/005; C07K 16/28

(52) U.S. Cl. CPC A61K 39/215; A61K 47/6901; C07K 14/005; C07K 16/2803; C07K 16/2827; C07K 16/2863; A61K 2039/55555; C07K 2317/24; C07K 2317/77; C07K 2317/92; C12N 2770/20022; C12N 2770/20034

(57) ABSTRACT

Described herein are compositions of extracellular vesicles, and methods and systems of producing the extracellular vesicles. Also described herein are methods of using the extracellular vesicles.

### CROSS REFERENCE TO RELATED APPLICATIONS

This application claims the benefit of U.S. Provisional Patent Application No. 63/061,749 filed on Aug. 5, 2020. Priority is claimed pursuant to 35 U.S.C. § 119. The above noted patent application is incorporated by reference as if set forth fully herein.

### INCORPORATION BY REFERENCE

All publications, patents, and patent applications mentioned in this specification are herein incorporated by reference, in their entireties, to the same extent as if each individual publication, patent, or patent application was specifically and individually indicated to be incorporated by reference.

### BACKGROUND

Extracellular vesicles are secreted by a wide variety of cell types. In general, extracellular vesicles such as exosomes, microvesicles, and apoptotic bodies are membrane-bound and can be loaded with a therapeutic cargo. For example, exosomes are a type of membrane-bound extracellular vesicle that are secreted by most eukaryotic cells. Exosome biogenesis may begin with pinching off of endosomal invaginations into the multivesicular body, forming intraluminal vesicles. If the multivesicular body fuses with the plasma membrane of the cell, the intraluminal vesicles may be released as exosomes. Microvesicles are budded out from a cell membrane surface. Apoptotic bodies, on the other hand, are released from dead cells. Exosomes, microvesicles, and apoptotic bodies can be released in vivo or in vitro, such as in cell-culture.

Extracellular vesicles have been explored as a vehicle for encapsulating and delivering therapeutics. Directing the extracellular vesicles to a target is generally challenging, as the majority of the extracellular vesicles are degraded in the liver, spleen, and/or kidney. Also, designing and manufacturing extracellular vesicles for encapsulating therapeutics for targeted delivery is time-consuming and expensive. For example, an extracellular vesicle designed for targeting one cell type may not effectively target another cell type. Therefore, there remains a need for extracellular vesicle that can be readily modified to target multiple cell types. There also remains a need for extracellular vesicles that can encapsulate sufficient quantity and quality of therapeutics to be delivered to the targeted cell.

### SUMMARY

This disclosure provides extracellular vesicles designed to target a wide variety of cell-types, including different cells and organs within the body and cells associated with a disease or disorder. In some instances, the extracellular vesicles provided herein can be readily modified to specifically bind to a target. For example, they may contain an extracellular domain (e.g., an extracellular domain of a transmembrane protein within the membrane of the extracellular vesicle) that binds to a cell-surface marker. In general, the extracellular vesicles provided herein comprise an adapter polypeptide with an extracellular domain and optionally a transmembrane domain that binds to a cell-surface marker.

Disclosed herein, in some aspects, are compositions comprising at least one extracellular vesicle, said extracellular vesicle comprising: at least one adapter polypeptide comprising a peptide sequence that binds to an Fc region of an antibody with a dissociation constant (Kd) of less than or equal to 10⁻⁹M, wherein said adapter polypeptide comprises an extracellular domain; said antibody complexed with said adapter polypeptide, wherein said antibody binds a first cell-surface marker associated with a diseased cell; and at least one therapeutic. Described herein, in some aspects, are compositions comprising at least one extracellular vesicle, said extracellular vesicle comprising: at least one adapter polypeptide comprising a peptide sequence that is at least 70% identical to a Fc receptor that specifically recognizes a Fc region of an antibody, wherein said adapter polypeptide comprises an extracellular domain; said antibody complexed with said adapter polypeptide, wherein said antibody binds a first cell-surface marker associated with a diseased cell; and at least one therapeutic. In some aspects, said Fc receptor is a Fc-gamma receptor, Fc-alpha receptor, or Fc-epsilon receptor. In some aspects, said Fc receptor comprises FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said diseased cell, wherein said targeting domain is attached to said extracellular domain of said adapter polypeptide. In some aspects, said targeting domain is selected from the group consisting of a tumor homing peptide, a tumor targeting domain, a tissue-targeting domain, a cell-penetrating peptide, a viral membrane protein, and any combination or fragment thereof. In some aspects, said diseased cell is a cancer cell or a non-cancerous lesion cell. In some aspects, said first cell-surface marker comprises EGFR, PD-L1, or ROR1. In some aspects, said first cell-surface marker and said second cell-surface marker are different. In some aspects, said first cell-surface marker and said second cell-surface marker are identical. In some aspects, said antibody is a humanized monoclonal antibody. In some aspects, said antibody is selected from the group consisting of humanized anti-EGFR antibody clone C225, humanized anti-ROR1 antibody clone 2A2, and humanized anti-PD-L1 antibody clone SP142. In some aspects, said humanized monoclonal antibody comprises an IgG. In some aspects, said humanized monoclonal antibody comprises an IgG1 or IgG3. In some aspects, said antibody is non-covalently complexed with said adapter polypeptide. In some aspects, said Fc region of said antibody is configured to complex to said adapter polypeptide in an acidic environment. In some aspects, said Fc region of said antibody is configured to be released from complexes to said adapter polypeptide in an acidic environment. In some aspects, said at least one therapeutic is within said extracellular vesicle. In some aspects, said at least one therapeutic is expressed on an extracellular surface of said extracellular vesicle. In some aspects, said at least one therapeutic is attached to said extracellular domain. In some aspects, said at least one therapeutic comprises a therapeutic polynucleotide, a therapeutic polypeptide, a therapeutic compound, a cancer drug, or a combination thereof. In some aspects, said therapeutic polynucleotide comprises a messenger RNA, a microRNA, a shRNA, or a combination thereof. In some aspects, said extracellular vesicle is an exosome, a microvesicle, or an apoptotic body. In some aspects, said extracellular vesicle is an exosome.

Described herein, in some instances, are methods of treating a subject, said methods comprising administering a therapeutically effective amount of a pharmaceutical composition to said subject, wherein said pharmaceutical composition comprises the compositions described herein. In some aspects, said pharmaceutical composition comprises at least one pharmaceutically acceptable excipient. In some aspects, said subject has cancer or a non-cancerous lesion. In some aspects, said subject has glioma. In some aspects, subject has muscular dystrophy. In some aspects, said muscular dystrophy is selected from the group consisting of Duchenne muscular dystrophy, Becker muscular dystrophy, facioscapulohumeral muscular dystrophy, congenital muscular dystrophy, and myotonic dystrophy. In some aspects, said subject has a retinal disease. In some aspects, said retinal disease is retinitis pigmentosa or Leber's congenital amaurosis. In some aspects, said therapeutically effective amount of said pharmaceutical composition comprises a therapeutically effective dose. In some aspects, said subject is administered said therapeutically effective amount of said pharmaceutical composition at a therapeutically effective frequency. In some aspects, said subject is administered said therapeutically effective amount of said pharmaceutical composition at a frequency of at least once per year. In some aspects, said subject is administered said therapeutically effective amount of said pharmaceutical composition at a frequency of at least once every six months. In some aspects, said subject is administered said therapeutically effective amount of said pharmaceutical composition at a frequency of at least once per month. In some aspects, said subject is administered said therapeutically effective amount of said pharmaceutical composition at a frequency of at least once per week. In some aspects, said pharmaceutical composition is an aqueous formulation. In some aspects, said pharmaceutical composition is formulated for injection. In some aspects, said pharmaceutical composition is administered to said subject intraventricularly, intracerebrally, intracerebellarly, intracerebroventricularly, intraperenchymally, subcutaneously, or a combination thereof.

Described herein, in some cases, are methods of producing a composition, said methods comprising: transfecting an extracellular vesicle donor cell with at least one heterologous polynucleotide encoding an adapter polypeptide, wherein said adapter polypeptide comprises a peptide sequence that is at least 70% identical to a Fc receptor, wherein said Fc receptor recognizes a Fc region of an antibody; collecting an extracellular vesicle released from said extracellular vesicle donor cell, wherein said extracellular vesicle released from said extracellular vesicle donor cell expresses said adapter polypeptide, wherein said adapter polypeptide comprises an extracellular domain, and wherein said extracellular vesicle comprises at least one therapeutic; and complexing said antibody to said extracellular domain, wherein said antibody binds a first cell-surface marker associated with a diseased cell. In some aspects, said Fc receptor is a Fc-gamma receptor, Fc-alpha receptor, or Fc-epsilon receptor. In some aspects, said Fc receptor is FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said diseased cell, wherein said targeting domain is attached to said extracellular domain. In some aspects, said first cell-surface marker or said second cell-surface marker is associated with a cancer cell or a non-cancerous lesion cell. In some aspects, said first cell-surface marker comprises EGFR, PD-L1, or ROR1. In some aspects, said first cell-surface marker and said second cell-surface marker are different. In some aspects, said first cell-surface marker and said second cell-surface markers are identical. In some aspects, said targeting domain is selected from the group consisting of a tumor homing peptide, a tumor targeting domain, a tissue-targeting domain, a cell-penetrating peptide, a viral membrane protein, and any combination or fragment thereof. In some aspects, said at least one therapeutic is within said extracellular vesicle. In some aspects, said at least one therapeutic is expressed on an extracellular surface of said extracellular vesicle. In some aspects, said at least one therapeutic is attached to said extracellular domain. In some aspects, said at least one therapeutic comprises a therapeutic polynucleotide, a therapeutic polypeptide, a therapeutic compound, a cancer drug, or a combination thereof. In some aspects, said therapeutic polynucleotide comprises a messenger RNA, a microRNA, a shRNA, or a combination thereof. In some aspects, said extracellular vesicle released from said extracellular vesicle donor cell is an exosome, a microvesicle, or an apoptotic body. In some aspects, said extracellular vesicle released from said extracellular vesicle donor cell is an exosome. In some aspects, said extracellular vesicle donor cell comprises electroporation, microfluidic electroporation, microchannel electroporation, or nanochannel electroporation. In some aspects, said microchannel electroporation or said nanochannel electroporation comprises use of micropore patterned silicon wafers, nanopore patterned silicon wafers, track etch membranes, ceramic micropore membranes, ceramic nanopore membranes, other porous materials, or a combination thereof. In some aspects, transfecting said extracellular vesicle donor cell comprises nanochannel electroporation, and wherein said at least one heterologous polynucleotide is nanoelectroporated into said extracellular vesicle donor cell via a nanochannel located on a biochip. In some aspects, transfecting said extracellular vesicle donor cell comprises use of a gene gun, micro-needle array, nano-needle array, sonication, or chemical permeation. In some aspects, said at least one heterologous polynucleotide is a plasmid.

Described herein, in some cases, are compositions comprising at least one extracellular vesicle, comprising: at least one adapter polypeptide comprising a peptide sequence that binds to an Fc region of an antibody with a dissociation constant (Kd) of less than or equal to 10⁻⁹M, wherein said adapter polypeptide comprises an extracellular domain; said antibody complexed with said adapter polypeptide, wherein said antibody specifically binds a first cell-surface marker associated with an immune cell; and at least one viral mimic peptide. Described herein, in some cases, is a composition comprising at least one extracellular vesicle, comprising: at least one adapter polypeptide comprising a peptide sequence that is at least 70% identical to a Fc receptor that binds to an Fc region of an antibody, wherein said adapter polypeptide comprises an extracellular domain; said antibody complexed with said adapter polypeptide, wherein said antibody specifically binds a first cell-surface marker associated with an immune cell; and at least one viral mimic peptide. In some aspects, said Fc receptor is a Fc-gamma receptor, Fc-alpha receptor, or Fc-epsilon receptor. In some aspects, said Fc receptor comprises FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said immune cell, wherein said targeting domain is attached to said extracellular domain of said adapter polypeptide. In some aspects, said immune cell is a T cell, a B cell, a dendritic cell, a macrophage, or a natural killer (NK) cell. In some aspects, said first cell-surface marker comprises LILRA4, CD3, CD19, CD20, or CD28. In some aspects, said first cell-surface marker and said second cell-surface marker are different. In some aspects, said first cell-surface marker and said second cell-surface marker are identical. In some aspects, said antibody is a humanized monoclonal antibody. In some aspects, said antibody is an IgG. In some aspects, said antibody is an IgG1 or IgG3. In some aspects, said antibody is non-covalently complexed with said adapter polypeptide. In some aspects, said Fc region of said antibody is configured to complex to said adapter polypeptide in an acidic environment. In some aspects, said Fc region of said antibody is configured to be released from complexed to said adapter polypeptide in an acidic environment. In some aspects, said at least one viral mimic peptide is expressed on an extracellular surface of said extracellular vesicle. In some aspects, said at least one viral mimic peptide is attached to said extracellular domain. In some aspects, said at least one viral mimic peptide comprises a peptide sequence that is at least 70% identical with a SARS-COV-2 viral protein. In some aspects, said SARS-COV-2 viral protein comprises an Envelopment (E) protein, a Nucleocapsid (N) protein, a Membrane (M) protein, or a Spike (S) protein. In some aspects, said SARS-COV-2 viral protein is said S protein. In some aspects, said extracellular vesicle comprises an exosome, a microvesicle, or an apoptotic body. In some aspects, said extracellular vesicle is an exosome.

Described herein, in some cases, are methods of vaccinating a subject, said methods comprising administering a therapeutically effective amount of a pharmaceutical composition to said subject, wherein said pharmaceutical composition comprises a composition described herein. In some aspects, said pharmaceutical composition comprises at least one pharmaceutically acceptable excipient. In some aspects, said therapeutically effective amount of said pharmaceutical composition comprises a therapeutically effective dose. In some aspects, said subject is administered said therapeutically effective amount of said pharmaceutical composition at a therapeutically effective frequency. In some aspects, said subject is administered said therapeutically effective amount of said pharmaceutical composition at a frequency of at least once per year. In some aspects, said subject is administered said therapeutically effective amount of said pharmaceutical composition at a frequency of at least once every six months. In some aspects, said subject is administered said therapeutically effective amount of said pharmaceutical composition at a frequency of at least once per month. In some aspects, said subject is administered said therapeutically effective amount of said pharmaceutical composition at a frequency of at least once per week. In some aspects, said pharmaceutical composition is an aqueous formulation. In some aspects, said pharmaceutical composition is formulated for injection. In some aspects, said pharmaceutical composition is administered to said subject intranasally, intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

Described herein, in some cases, are methods of producing a composition described herein, said methods comprising: transfecting an extracellular vesicle donor cell with at least one heterologous polynucleotide encoding an adapter polypeptide, wherein said adapter polypeptide comprises a peptide sequence that is at least 70% identical to a Fc receptor, wherein said Fc receptor recognizes a Fc region of an antibody; collecting an extracellular vesicle released from said extracellular vesicle donor cell, wherein said extracellular vesicle released from said extracellular vesicle donor cell expresses said adapter polypeptide, wherein said adapter polypeptide comprises an extracellular domain, and wherein said extracellular vesicle comprises at least one viral mimic peptide; and complexing said antibody to said extracellular domain, wherein said antibody binds a first cell-surface marker associated with an immune cell. In some aspects, said Fc receptor is a Fc-gamma receptor, Fc-alpha receptor, or Fc-epsilon receptor. In some aspects, said Fc receptor comprises FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said immune cell, wherein said targeting domain is attached to said extracellular domain. In some aspects, said immune cell is a T cell, a B cell, a dendritic cell, a macrophage, or a natural killer (NK) cell. In some aspects, said first cell-surface marker comprises LILRA4, CD3, CD19, CD20, or CD28. In some aspects, said first cell-surface marker and said second cell-surface marker are different. In some aspects, said first cell-surface marker and said second cell-surface markers are identical. In some aspects, said antibody comprises a humanized monoclonal antibody. In some aspects, said antibody is an IgG. In some aspects, said antibody is an IgG1 or IgG3. In some aspects, said antibody is non-covalently complexed with said adapter polypeptide. In some aspects, said Fc region of said antibody is configured to complex to said adapter polypeptide in an acidic environment. In some aspects, said Fc region of said antibody is configured to be released from complexed to said adapter polypeptide in an acidic environment. In some aspects, said at least one viral mimic peptide is expressed on an extracellular surface of said extracellular vesicle. In some aspects, said at least one viral mimic peptide is attached to said extracellular domain. In some aspects, said at least one viral mimic peptide comprises a peptide sequence that is at least 70% identical with a SARS-COV-2 viral protein. In some aspects, said SARS-COV-2 viral protein comprises an Envelopment (E) protein, a Nucleocapsid (N) protein, a Membrane (M) protein, or a Spike (S) protein. In some aspects, said SARS-COV-2 viral protein is said S protein. In some aspects, said extracellular vesicle comprises an exosome, a microvesicle, or an apoptotic body. In some aspects, said extracellular vesicle is an exosome. In some aspects, transfecting said extracellular vesicle donor cell comprises electroporation, microfluidics electroporation, microchannel electroporation, or nanochannel electroporation. In some aspects, said microchannel electroporation or said nanochannel electroporation comprises use of micropore patterned silicon wafers, nanopore patterned silicon wafers, track etch membranes, ceramic micropore membranes, ceramic nanopore membranes, other porous materials, or a combination thereof. In some aspects, transfecting said extracellular vesicle donor cell comprises nanochannel electroporation, and wherein said at least one heterologous polynucleotide is nanoelectroporated into said extracellular vesicle donor cell via a nanochannel located on a biochip. In some aspects, transfecting said extracellular vesicle donor cell comprises a use of a gene gun, micro-needle array, nano-needle array, sonication, or chemical permeation. In some aspects, said at least one heterologous polynucleotide is a plasmid.

Disclosed herein, in some aspects, are compositions comprising at least one extracellular vesicle, said extracellular vesicle comprising: at least one adapter polypeptide comprising a peptide sequence that binds to an Fc region of a binding molecule with a dissociation constant (Kd) of less than or equal to 10⁻⁹M, wherein said adapter polypeptide comprises an extracellular domain; said binding molecule complexed with said adapter polypeptide, wherein said binding molecule binds a first cell-surface marker associated with a diseased cell; and at least one therapeutic. Described herein, in some aspects, are compositions comprising at least one extracellular vesicle, said extracellular vesicle comprising: at least one adapter polypeptide comprising a peptide sequence that is at least 70% identical to a Fc receptor that specifically recognizes a Fc region of a binding molecule, wherein said adapter polypeptide comprises an extracellular domain; said binding molecule complexed with said adapter polypeptide, wherein said binding molecule binds a first cell-surface marker associated with a diseased cell; and at least one therapeutic. In some aspects, said Fc receptor is a Fc-gamma receptor, Fc-alpha receptor, or Fc-epsilon receptor. In some aspects, said Fc receptor comprises FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said diseased cell, wherein said targeting domain is attached to said extracellular domain of said adapter polypeptide. In some aspects, said targeting domain is selected from the group consisting of a tumor homing peptide, a tumor targeting domain, a tissue-targeting domain, a cell-penetrating peptide, a viral membrane protein, and any combination or fragment thereof. In some aspects, said diseased cell is a cancer cell or a non-cancerous lesion cell. In some aspects, said first cell-surface marker comprises EGFR, PD-L1, or ROR1. In some aspects, said first cell-surface marker and said second cell-surface marker are different. In some aspects, said first cell-surface marker and said second cell-surface marker are identical. In some aspects, said binding molecule is a humanized monoclonal antibody. In some aspects, said binding molecule is selected from the group consisting of humanized anti-EGFR antibody clone C225, humanized anti-ROR1 antibody clone 2A2, and humanized anti-PD-L1 antibody clone SP142. In some aspects, said humanized monoclonal antibody comprises an IgG. In some aspects, said humanized monoclonal antibody comprises an IgG1 or IgG3. In some aspects, said binding molecule is non-covalently complexed with said adapter polypeptide. In some aspects, said Fc region of said binding molecule is configured to complex to said adapter polypeptide in an acidic environment. In some aspects, said Fc region of said binding molecule is configured to be released from complexes to said adapter polypeptide in an acidic environment. In some aspects, said at least one therapeutic is within said extracellular vesicle. In some aspects, said at least one therapeutic is expressed on an extracellular surface of said extracellular vesicle. In some aspects, said at least one therapeutic is attached to said extracellular domain. In some aspects, said at least one therapeutic comprises a therapeutic polynucleotide, a therapeutic polypeptide, a therapeutic compound, a cancer drug, or a combination thereof. In some aspects, said therapeutic polynucleotide comprises a messenger RNA, a microRNA, a shRNA, or a combination thereof. In some aspects, said extracellular vesicle is an exosome, a microvesicle, or an apoptotic body. In some aspects, said extracellular vesicle is an exosome.

Described herein, in some instances, are methods of treating a subject, said methods comprising administering a therapeutically effective amount of a pharmaceutical composition to said subject, wherein said pharmaceutical composition comprises the compositions described herein. In some aspects, said pharmaceutical composition comprises at least one pharmaceutically acceptable excipient. In some aspects, said subject has cancer or a non-cancerous lesion. In some aspects, said subject has glioma. In some aspects, subject has muscular dystrophy. In some aspects, said muscular dystrophy is selected from the group consisting of Duchenne muscular dystrophy, Becker muscular dystrophy, facioscapulohumeral muscular dystrophy, congenital muscular dystrophy, and myotonic dystrophy. In some aspects, said subject has a retinal disease. In some aspects, said retinal disease is retinitis pigmentosa or Leber's congenital amaurosis. In some aspects, said therapeutically effective amount of said pharmaceutical composition comprises a therapeutically effective dose. In some aspects, said subject is administered said therapeutically effective amount of said pharmaceutical composition at a therapeutically effective frequency. In some aspects, said subject is administered said therapeutically effective amount of said pharmaceutical composition at a frequency of at least once per year. In some aspects, said subject is administered said therapeutically effective amount of said pharmaceutical composition at a frequency of at least once every six months. In some aspects, said subject is administered said therapeutically effective amount of said pharmaceutical composition at a frequency of at least once per month. In some aspects, said subject is administered said therapeutically effective amount of said pharmaceutical composition at a frequency of at least once per week. In some aspects, said pharmaceutical composition is an aqueous formulation. In some aspects, said pharmaceutical composition is formulated for injection. In some aspects, said pharmaceutical composition is administered to said subject intraventricularly, intracerebrally, intracerebellarly, intracerebroventricularly, intraperenchymally, subcutaneously, or a combination thereof.

Described herein, in some cases, are methods of producing a composition, said methods comprising: transfecting an extracellular vesicle donor cell with at least one heterologous polynucleotide encoding an adapter polypeptide, wherein said adapter polypeptide comprises a peptide sequence that is at least 70% identical to a Fc receptor, wherein said Fc receptor recognizes a Fc region of a binding molecule; collecting an extracellular vesicle released from said extracellular vesicle donor cell, wherein said extracellular vesicle released from said extracellular vesicle donor cell expresses said adapter polypeptide, wherein said adapter polypeptide comprises an extracellular domain, and wherein said extracellular vesicle comprises at least one therapeutic; and complexing said binding molecule to said extracellular domain, wherein said binding molecule binds a first cell-surface marker associated with a diseased cell. In some aspects, said Fc receptor is a Fc-gamma receptor, Fc-alpha receptor, or Fc-epsilon receptor. In some aspects, said Fc receptor is FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said diseased cell, wherein said targeting domain is attached to said extracellular domain. In some aspects, said first cell-surface marker or said second cell-surface marker is associated with a cancer cell or a non-cancerous lesion cell. In some aspects, said first cell-surface marker comprises EGFR, PD-L1, or ROR1. In some aspects, said first cell-surface marker and said second cell-surface marker are different. In some aspects, said first cell-surface marker and said second cell-surface markers are identical. In some aspects, said targeting domain is selected from the group consisting of a tumor homing peptide, a tumor targeting domain, a tissue-targeting domain, a cell-penetrating peptide, a viral membrane protein, and any combination or fragment thereof. In some aspects, said at least one therapeutic is within said extracellular vesicle. In some aspects, said at least one therapeutic is expressed on an extracellular surface of said extracellular vesicle. In some aspects, said at least one therapeutic is attached to said extracellular domain. In some aspects, said at least one therapeutic comprises a therapeutic polynucleotide, a therapeutic polypeptide, a therapeutic compound, a cancer drug, or a combination thereof. In some aspects, said therapeutic polynucleotide comprises a messenger RNA, a microRNA, a shRNA, or a combination thereof. In some aspects, said extracellular vesicle released from said extracellular vesicle donor cell is an exosome, a microvesicle, or an apoptotic body. In some aspects, said extracellular vesicle released from said extracellular vesicle donor cell is an exosome. In some aspects, said extracellular vesicle donor cell comprises electroporation, microfluidic electroporation, microchannel electroporation, or nanochannel electroporation. In some aspects, said microchannel electroporation or said nanochannel electroporation comprises use of micropore patterned silicon wafers, nanopore patterned silicon wafers, track etch membranes, ceramic micropore membranes, ceramic nanopore membranes, other porous materials, or a combination thereof. In some aspects, transfecting said extracellular vesicle donor cell comprises nanochannel electroporation, and wherein said at least one heterologous polynucleotide is nanoelectroporated into said extracellular vesicle donor cell via a nanochannel located on a biochip. In some aspects, transfecting said extracellular vesicle donor cell comprises use of a gene gun, micro-needle array, nano-needle array, sonication, or chemical permeation. In some aspects, said at least one heterologous polynucleotide is a plasmid.

Described herein, in some cases, are composition comprising at least one extracellular vesicle, comprising: at least one adapter polypeptide comprising a peptide sequence that binds to an Fc region of a binding molecule with a dissociation constant (Kd) of less than or equal to 10⁻⁹M, wherein said adapter polypeptide comprises an extracellular domain; said binding molecule complexed with said adapter polypeptide, wherein said binding molecule specifically binds a first cell-surface marker associated with an immune cell; and at least one viral mimic peptide. Described herein, in some cases, is a composition comprising at least one extracellular vesicle, comprising: at least one adapter polypeptide comprising a peptide sequence that is at least 70% identical to a Fc receptor that binds to an Fc region of a binding molecule, wherein said adapter polypeptide comprises an extracellular domain; said binding molecule complexed with said adapter polypeptide, wherein said binding molecule specifically binds a first cell-surface marker associated with an immune cell; and at least one viral mimic peptide. In some aspects, said Fc receptor is a Fc-gamma receptor, Fc-alpha receptor, or Fc-epsilon receptor. In some aspects, said Fc receptor comprises FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said immune cell, wherein said targeting domain is attached to said extracellular domain of said adapter polypeptide. In some aspects, said immune cell is a T cell, a B cell, a dendritic cell, a macrophage, or a natural killer (NK) cell. In some aspects, said first cell-surface marker comprises LILRA4, CD3, CD19, CD20, or CD28. In some aspects, said first cell-surface marker and said second cell-surface marker are different. In some aspects, said first cell-surface marker and said second cell-surface marker are identical. In some aspects, said binding molecule is a humanized monoclonal antibody. In some aspects, said binding molecule is an IgG. In some aspects, said binding molecule is an IgG1 or IgG3. In some aspects, said binding molecule is non-covalently complexed with said adapter polypeptide. In some aspects, said Fc region of said binding molecule is configured to complex to said adapter polypeptide in an acidic environment. In some aspects, said Fc region of said binding molecule is configured to be released from complexed to said adapter polypeptide in an acidic environment. In some aspects, said at least one viral mimic peptide is expressed on an extracellular surface of said extracellular vesicle. In some aspects, said at least one viral mimic peptide is attached to said extracellular domain. In some aspects, said at least one viral mimic peptide comprises a peptide sequence that is at least 70% identical with a SARS-COV-2 viral protein. In some aspects, said SARS-COV-2 viral protein comprises an Envelopment (E) protein, a Nucleocapsid (N) protein, a Membrane (M) protein, or a Spike (S) protein. In some aspects, said SARS-COV-2 viral protein is said S protein. In some aspects, said extracellular vesicle comprises an exosome, a microvesicle, or an apoptotic body. In some aspects, said extracellular vesicle is an exosome.

Described herein, in some cases, are methods of vaccinating a subject, said methods comprising administering a therapeutically effective amount of a pharmaceutical composition to said subject, wherein said pharmaceutical composition comprises a composition described herein. In some aspects, said pharmaceutical composition comprises at least one pharmaceutically acceptable excipient. In some aspects, said therapeutically effective amount of said pharmaceutical composition comprises a therapeutically effective dose. In some aspects, said subject is administered said therapeutically effective amount of said pharmaceutical composition at a therapeutically effective frequency. In some aspects, said subject is administered said therapeutically effective amount of said pharmaceutical composition at a frequency of at least once per year. In some aspects, said subject is administered said therapeutically effective amount of said pharmaceutical composition at a frequency of at least once every six months. In some aspects, said subject is administered said therapeutically effective amount of said pharmaceutical composition at a frequency of at least once per month. In some aspects, said subject is administered said therapeutically effective amount of said pharmaceutical composition at a frequency of at least once per week. In some aspects, said pharmaceutical composition is an aqueous formulation. In some aspects, said pharmaceutical composition is formulated for injection. In some aspects, said pharmaceutical composition is administered to said subject intranasally, intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

Described herein, in some cases, are methods of producing a composition described herein, said methods comprising: transfecting an extracellular vesicle donor cell with at least one heterologous polynucleotide encoding an adapter polypeptide, wherein said adapter polypeptide comprises a peptide sequence that is at least 70% identical to a Fc receptor, wherein said Fc receptor recognizes a Fc region of a binding molecule; collecting an extracellular vesicle released from said extracellular vesicle donor cell, wherein said extracellular vesicle released from said extracellular vesicle donor cell expresses said adapter polypeptide, wherein said adapter polypeptide comprises an extracellular domain, and wherein said extracellular vesicle comprises at least one viral mimic peptide; and complexing said binding molecule to said extracellular domain, wherein said binding molecule binds a first cell-surface marker associated with an immune cell. In some aspects, said Fc receptor is a Fc-gamma receptor, Fc-alpha receptor, or Fc-epsilon receptor. In some aspects, said Fc receptor comprises FcγRI (CD64), FcγRII (CD32), or FcγRIII (CD16). In some aspects, said Fc receptor is CD64. In some aspects, said adapter polypeptide further comprises a targeting domain that binds a second cell-surface marker associated with said immune cell, wherein said targeting domain is attached to said extracellular domain. In some aspects, said immune cell is a T cell, a B cell, a dendritic cell, a macrophage, or a natural killer (NK) cell. In some aspects, said first cell-surface marker comprises LILRA4, CD3, CD19, CD20, or CD28. In some aspects, said first cell-surface marker and said second cell-surface marker are different. In some aspects, said first cell-surface marker and said second cell-surface markers are identical. In some aspects, said binding molecule comprises a humanized monoclonal antibody. In some aspects, said binding molecule is an IgG. In some aspects, said binding molecule is an IgG1 or IgG3. In some aspects, said binding molecule is non-covalently complexed with said adapter polypeptide. In some aspects, said Fc region of said binding molecule is configured to complex to said adapter polypeptide in an acidic environment. In some aspects, said Fc region of said binding molecule is configured to be released from complexed to said adapter polypeptide in an acidic environment. In some aspects, said at least one viral mimic peptide is expressed on an extracellular surface of said extracellular vesicle. In some aspects, said at least one viral mimic peptide is attached to said extracellular domain. In some aspects, said at least one viral mimic peptide comprises a peptide sequence that is at least 70% identical with a SARS-COV-2 viral protein. In some aspects, said SARS-COV-2 viral protein comprises an Envelopment (E) protein, a Nucleocapsid (N) protein, a Membrane (M) protein, or a Spike (S) protein. In some aspects, said SARS-COV-2 viral protein is said S protein. In some aspects, said extracellular vesicle comprises an exosome, a microvesicle, or an apoptotic body. In some aspects, said extracellular vesicle is an exosome. In some aspects, transfecting said extracellular vesicle donor cell comprises electroporation, microfluidics electroporation, microchannel electroporation, or nanochannel electroporation. In some aspects, said microchannel electroporation or said nanochannel electroporation comprises use of micropore patterned silicon wafers, nanopore patterned silicon wafers, track etch membranes, ceramic micropore membranes, ceramic nanopore membranes, other porous materials, or a combination thereof. In some aspects, transfecting said extracellular vesicle donor cell comprises nanochannel electroporation, and wherein said at least one heterologous polynucleotide is nanoelectroporated into said extracellular vesicle donor cell via a nanochannel located on a biochip. In some aspects, transfecting said extracellular vesicle donor cell comprises a use of a gene gun, micro-needle array, nano-needle array, sonication, or chemical permeation. In some aspects, said at least one heterologous polynucleotide is a plasmid.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative aspects.

FIG. 1 depicts a schematic of a targeting extracellular vesicle (“EXO”) comprising a monoclonal antibody (mAb) and a tumor homing peptide (THP) linked to a CD64 on the extracellular vesicle surface. These extracellular vesicles (EVs) can target tumors and lesions, with exemplary targets recited in FIG. 1. The EVs with CD64 or THP-CD64 can be generated by transfection of donor cells with human CD64 plasmid DNA or human THP-CD64 plasmid DNA to express either human CD64 or human THP-CD64 on the surface of EVs (including exosomes) secreted from transfected donor cells. CD64 provides a biological anchor for binding to a humanized monoclonal antibody (hmAB). The extracellular D1-D2 hinge of human CD64 binds to the lower hinge region of Fc in human IgG1 with high affinity (a dissociation constant (K_{d}) as high as ˜10⁻⁹ M (nanomolar). In addition to the ability of the bound hmAb to specifically recognize cell targets, targeting by a small tumor homing peptide (THP) can also be engineered onto the N-terminal of CD64. Dual targeting of both hmAbs and THPs on the EV (or exosome) surface enhances targeting and delivery to tumors and other lesions in vivo. Examples of hmAbs for cancer/tumor targeting include, but are not limited to, anti-hEGFR such as Cetuximab, anti-hPD-L1 such as Atezolizumab, and anti-humanized ROR1. Examples of THPs for cancer/tumor targeting include, but are not limited to, CKAAKN (CK), CREKA (CR), and ARRPKLD (AR).

FIGS. 2A-2D illustrates an exemplary construct design for plasmids encoding CD64 with additional tumor homing peptides. FIG. 2A. Plasmids were constructed with the vector carrying genes for Ampicillin resistance (AmpR) and the EGFR marker for transformation and transfection, respectively. The functional CD64 was encoded by the coding sequence of CD64 (CD64_CDS) driven by the EF-1αpromoter. FIG. 2B. The CD64_CDS (355 amino acids) included a (i) signal peptide (SP), (ii) an extracellular region (D1, D2, and D3), (iii) a transmembrane (TM) region, and (iv) an intracellular (IC) domain, and the THPs were inserted into the gap between the signal peptide and the extracellular D1 region, allowing expression of the THP at the N-terminus of CD64. FIG. 2C. The THPs were connected by a Flag (DYKDDDK) linker to the N-terminus of extracellular D1, limiting the conformational block of the Fc binding region at the D1-D2 hinge of CD64. FIG. 2D. List of exemplary peptide and nucleotide sequences of selected THPs [Flag_control, CKAAKN (CK), CREKA (CR), and ARRPKLD (AR)].

FIGS. 3A-3C illustrates how addition of the THP onto CD64 does not affect binding affinity with human IgG (hIgG). FIG. 3A. Schematic of purified CD64 proteins with different THPs bound to the immobilized hIgG on a solid support and reacted with ELISA substrates. The K_{d} value was determined by the monovalent modeling between CD64 and hIgG. FIG. 3B. The affinity index K_{d} of hIgG and recombinant wild-type CD64 (wt_CD64) was measured. FIG. 3C. The affinity index K_{d} of different engineered THP-CD64 proteins with hIgG suggested that the engineered CD64 with different THPs (Flag, CK, CR, AR) did not affect the high binding affinity with mAb in nM-level comparing to wt_CD64.

FIGS. 4A-4B illustrates EV number and content of endogenous mRNA from nanochannel electroporation (NEP) transfected mouse embryonic fibroblasts (MEFs) with THP-CD64 and therapeutic RNA plasmids. FIG. 4A. EV number per cell produced by untreated MEFs in PBS (control group), MEFs transfected with both THP-CD64 and human TP53 plasmids by NEP, and MEFs transfected with both THP-CD64 and shKRAS G12D mutation plasmids by NEP at 24 h.

FIG. 4B. Fold change of TP53 mRNA in EVs produced by untreated and NEP-transfected MEFs by qRT-PCR.

FIGS. 5A-5D illustrates that THP-CD64 expressing exosomes retained high binding affinity with hIgG. FIG. 5A. Schematic of purified exosomes with engineered THP-CD64 were captured by latex beads and incubated with anti-CD64-APC, anti-CD63-BV510 and FITC-conjugated hIgG for flow cytometry assay. FIG. 5B. Profiling of surface expression followed the standard protocol to gate the singlet bead and CD63+ exosome population in order to determine mean fluorescence intensity (MFI) of CD64 expression and hIgG binding. FIG. 5C. Surface co-expression of CD64 within the CD63+ exosomal population was determined by MFI of FITC and confirmed the exosomal expression of engineered CD64 with either Flag, CK, CR or AR THP. FIG. 5D. Surface co-expression of hIgG and CD64 within the CD63+ exosomal population was determined by MFI of FITC and confirmed the high binding affinity of hIgG on exosomes expressing CD64 with either Flag, CK, CR or AR THP.

FIG. 6 illustrates uptake of liposome and EVs in cancer spheroids from a human pancreatic cancer cell line, PANC-1. The purified EVs released from mouse embryonic fibroblast (MEF) cells after transfection of either Flag-CD64 or CK-CD64 plasmid DNA (CK-CD64) were formulated with either humanized anti-EGFR mAb (Cetuximab) or hIgG. The cancer spheroids were treated with PKH67(green)-labeled liposome (lipofectamine 3000) and various EVs for 24 hours, and subsequently processed by fixation, permeation, and staining with anti-hIgG-TRITC (red) and DAPI (blue). The cross section of cancer spheroids was imaged under confocal microscopy. Cancer spheroid treatment with various EVs all showed better spheroid uptake than the commercial lipofectamine 3000 based on fluorescence intensity and distribution. Among various EVs, the dual targeting EV (CK-CD64-Cet_Exo) revealed the highest spheroid uptake.

FIGS. 7A-7C illustrates dual targeting of CK-CD64 and humanized anti-EGFR mAb (Cetuximab) enhances EV uptake in PANC-1 cancer spheroid cells, particularly the CD24+CD44+ subpopulation. FIG. 7A. The PANC-1 cancer spheroids were formed and cultured for a week to reach a diameter of ˜500 μm, then treated with ˜10⁹ PKH67-labled exosomes in culture media for 24 h. FIG. 7B. The treated spheroids were disassembled into single-cell suspension to identify the subpopulations by CD24 and CD44 expression using flow cytometry. FIG. 7C. The mean fluorescence intensity of PKH67 measured in CD24lowCD44low or CD24+CD44+ subpopulations represent their EV uptake. The engineered EVs containing Flag-CD64, CK-CD64, CR-CD64, or AR-CD64 with humanized antibody binding (Cetuximab: anti-EGFR, Atezolizumab: anti-PD-L1, or hIgG) all showed good cellular uptake, particularly for the CD44+CD24+ subpopulation. The dual targeting EVs with anti-hEGFR (Cetuximab) and CK-CD64 provided the best cellular uptake for both PANC-1 cell subpopulations.

FIG. 8 depicts that the uptake of extracellular vesicles is enhanced by targeting ROR1, which is highly expressed in 85% of pancreatic cancer, within spheroids formed from PANC-1. PANC-1 spheroids were formed and stably cultured for a week until a diameter of 300-500 μm was obtained, and then treated with 10\hat{ }10 PKH67-labeled exosomes in culture media for 24 hours.

FIG. 9 depicts the enhanced uptake of ROR1-targeted extracellular vesicles in vivo in a PANC-1 orthotopic model. Mice (4 weeks post xenograftment of PANC-1 extracellular vesicles) were treated with 1.0E12/50 μl (intraperitoneal) injection with extracellular vesicle solution (250 μl) and sacrificed after 24 hours. PKH26 (Excitation/Emission: 535/580 nm); GFP (465/540 nm); IVIS (Epi-illumination, Bin:(M)1, FOV:22, f2, 5s. Distribution: brain/heart/lung/liver/spleen/pancreas/kidney.

FIG. 10 depicts the enhanced uptake of ROR1-targeted extracellular vesicles by penetration through tumor tissue. Anti-ROR1 targeting enriches the extracellular vesicle uptake in tumor lesions, whereas the update of the CK_peptide extracellular vesicle is not significant compared to the flag control.

FIGS. 11A-11D illustrates an exemplary design of vacosomes and five proposed vaccine peptides (i.e. Spike, S-protein, fragments) from the epitope and structural predictions for COVID-19 vaccine development. FIGS. 11A. ACE2 acts as the receptor for the SARS-COV-2 virus and allows it to infect the cell. FIG. 11B. A strong vaccination through T-cell receptor (TCR) complex can be synergistically achieved by vaccination peptides on the N-terminal of CD64 and co-stimulation by the preloaded anti-&CD3/CD28 mAb on the hinge D1-D2 of CD64. Exosomes that overexpress various viral protein fragments fused to CD64 on the exosomal surface can serve as a vaccine (designated ‘vacosome’). FIG. 11C. The formation of an immunological synapse between engineered CD64 and TCR can be confirmed by the fluorescent tag and T-cell surface markers staining using fluorescence-activated cell sorter (FACS). Similarly, the co-loading of mAb targeting antigen presenting cells (APCs) such as B cells (anti-αCD19/CD20) and dendritic cells (DCs) (anti-αLILRA4) should enhance APC-T cell responses. FIG. 11D. Five fusion S-protein fragment candidates that have high potential to serve as a vaccine peptide for COVID-19 are selected from the epitope and structural predictions. They can be expressed on vacosomes generated via NEP transfected donor cells such as human mesenchymal stem cells (MSCs) and DCs.

FIGS. 12A-12B illustrates binding affinity strength of human immunoglobins and classical Fc receptors. Fc receptors embedded in the plasma membrane contain intracellular domains or subunits that can trigger a downstream activation or suppression. FIG. 12A. IgG affinity-altering variants are highlighted with respective human Fcγ receptor members, from very high (deep orange), high (orange), medium (yellow), low (light blue), to no binding (dark blue). FcRn receptor binds to IgG subclasses under acidic conditions (e.g. pH-6) but decrease the binding ability in physiological conditions, pH=7.4. FIG. 12B. IgE has very high binding affinity with FcεRI receptor, but low affinity with FcεRII receptor. IgA has low binding affinity with FcαRI receptor. *** The binding affinity between human immunoglobins and Fc repeaters is shown as constant K_{d} at the level as Very High+++: ˜10⁻⁹ M; High++: 10⁻⁹ to 10⁻⁸ M; Medium: ˜ 10⁻⁷ M; or Low: >10⁻⁷ M.

FIGS. 13A-13D illustrate the dynamics of EV release in NEP. FIG. 13A depicts EVs secretion profiles over time after NEP. FIG. 13B depicts fold change of TP53 mRNA expression within the EVs over time was measured by qPCR. FIG. 13C depicts CD64 expression on EVs surface was measured through ELISA for EVs collected every 8 h. FIG. 13D shows the expression level of KRAS^{G12D} shRNA within the EVs every 8 h after NEP.

FIG. 14A-C illustrates sequential NEP (sNEP) designs for TP53 mRNA/CD64 EVs. FIG. 14A provides EV number and TP53 mRNA expression in the (FIG. 14A) 8-h, (FIG. 14B) 16-h, and (FIG. 14C) 24-h sNEP cases. Ctrl is one-time NEP with CD64 plasmid.

FIG. 15A-F provides characterization of the as-prepared targeting EVs (tEVs). FIG. 15A provides size distribution of blank EVs (Control) and engineered EVs obtained by NEP, (FIG. 15B) exosomal biomarkers on as-prepared EVs, (FIG. 15C) SEM and (FIG. 15D) CryoTEM images of representative EVs. (FIG. 15E) single EV capture and co-localization characterization using ILN biochips on TIRF microscope with fluorescence labelled anti-CD64 and molecular beacons for KRAS^{G12D} shRNA and TP53 mRNA, (FIG. 15F) ratios of EVs containing CD64 protein, KRAS^{G12D} shRNA, TP53 mRNA, and co-localization of CD64/KRAS^{G12D} shRNA and CD64/TP53 mRNA.

FIG. 16A-G depict that binding tumor-specific antibodies (αhROR1 and αhEGFR) on CD64/EV surface can enhance cellular internalization of EVs in PANC-1 cells. FIG. 16A provides the uptake efficiency of humanized antibodies on CD64 flag-peptide FIG. 16B provides the uptake efficiency of humanized antibodies on CD64 with CK-peptide. FIG. 16C quantifies the relative EV uptake by each formulation FIG. 16D compares staining of PANC-1 cells without treatment (Con), and with IgG_EV, αEGFR_EV, and αROR1_EV treatment for 4 hours. FIG. 16E-F provides an EV uptake assay on 3D tumor spheroids of PANC-1 cells for αEGFR_EVs (FIG. 16E) and αROR1_EVs (FIG. 16F). FIG. 16G provides a substitution assay with human serum (50%) for κ hours at 37° C.

FIG. 17A-D depict a TRANSWELL®-based transcytosis assay and results. FIG. 17A provides a schematic of the assay. FIG. 17B provides various inhibitors selected to block endocytosis and EV secretion (including pitstop 2, an inhibitor of clathrin-mediated endocytosis; methyl-β-cyclodextrin, an inhibitor of caveolae-mediated endocytosis; cytochalasin D, an inhibitor of micropinocytosis; and neticonazole, an inhibitor of exosomal secretion). FIG. 17C provides data from a transcytosis assay by PANC-1 using various inhibitors (“ctl”: 1E10 non-targeting EVs without inhibitors in upper PANC-1 cells; “Pos ctl”: no upper cellular layer). FIG. 17D compares transcytosis levels by PANC-1 using targeting hmAbs on the EV surface.

FIG. 18A-B demonstrates that human serum IgG does not affect human mAb on the EV surface. FIG. 18A-B depict that αhEGFR_EV (left panels) and αhROR1_EV (right panels) incubated with human serum (50%) for 6 h at 37° C., then treated with monolayer PANC-1 cells, maintained the same targeting ability after human serum incubation.

FIG. 19A-B depicts biodistribution of targeting EVs in a PANC-1 orthotopic NS mice. FIG. 19A depicts in vivo imaging (IVIS) and FIG. 19B depicts expression in brain, heart, lung, liver, spleen, pancreas, and kidney.

### DETAILED DESCRIPTION

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present aspects disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Any systems, methods, compositions, and platforms described herein are modular and not limited to sequential steps. Accordingly, terms such as “first” and “second” do not necessarily imply priority, order of importance, or order of acts.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “increased”, “increasing”, or “increase” are used herein to generally mean an increase by a statically significant amount. In some cases, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some cases, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. Other examples of “decrease” include a decrease of at least 2-fold, at least 5-fold, at least 10-fold, at least 20-fold, at least 50-fold, at least 100-fold, at least 1000-fold or more as compared to a reference level. The decrease can be, for example, at least 10%, at least 20%, at least 30%, at least 40% or more, and is preferably down to a level accepted as within the range of normal for an individual without a given disease.

As used herein, a “cell” generally refers to a biological cell. A cell is the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell is a synthetically made, sometimes termed an artificial cell). A cell can be derived from a cell line.

The terms “transfection” or “transfected” generally refers to introduction of a nucleic acid molecule into a cell by non-viral or viral-based methods. The nucleic acid molecules can be gene sequences encoding complete proteins or functional portions thereof. In some cases, the nucleic acid molecules can be non-coding sequences. In some cases, the transfection methods are utilized for introducing nucleic acid molecules into a cell for generating a transgenic animal. Such techniques can include pronuclear microinjection, retrovirus mediated gene transfer into germ lines, gene targeting into embryonic stem cells, electroporation of embryos, sperm mediated gene transfer, and in vitro transformation of somatic cells, such as cumulus or mammary cells, or adult, fetal, or embryonic stem cells, followed by nuclear transplantation.

“Nanoelectroporation” or “nanochannel electroporation” refers to transfecting a cell with at least one heterologous polynucleotide such as a vector by loading the at least one heterologous polynucleotide into a nanochannel and accelerating the at least on heterologous polynucleotide into the cell with by generating an electric field. The cell to be transfected is situated at an opening of the nanochannel, where the electric field of the nanoelectroporation creates pores in the cell membrane to allow the at least one heterologous polynucleotide to be introduced into the cell.

A “plasmid,” as used herein, generally refers to a non-viral expression vector, e.g., a nucleic acid molecule that encodes for genes and/or regulatory elements necessary for the expression of genes. The term “vector,” as used herein, generally refers to a nucleic acid molecule capable transferring or transporting a payload nucleic acid molecule. The payload nucleic acid molecule can be generally linked to, e.g., inserted into, the vector nucleic acid molecule. A vector can include sequences that direct autonomous replication in a cell, or can include sequences sufficient to allow integration into host cell gene (e.g., host cell DNA). Examples of a vector can include, but are not limited to, plasmids (e.g., DNA plasmids or RNA plasmids), transposons, cosmids, bacterial artificial chromosomes, and viral vectors. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to Gamma-retroviral, Alpha-retroviral, Foamy viral, lentiviral, adenoviral, or adeno-associated viral vectors. A vector of any of the embodiments of the present disclosure can comprise exogenous, endogenous, or heterologous control sequences such as promoters and/or enhancers.

The term “nucleotide,” as used herein, generally refers to a base-sugar-phosphate combination. A nucleotide can comprise a synthetic nucleotide. A nucleotide can comprise a synthetic nucleotide analog. Nucleotides are monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. In some cases, a polynucleotide is exogenous (e.g. a heterologous polynucleotide) to a cell. In some cases, a polynucleotide is endogenous to a cell. In some cases, a polynucleotide can exist in a cell-free environment. In some cases, a polynucleotide is a gene or fragment thereof. In some cases, a polynucleotide is DNA. In some cases, a polynucleotide is RNA. A polynucleotide can have any three dimensional structure, and can perform any function, known or unknown. In some cases, a polynucleotide comprises one or more analogs (e.g. altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), non-coding RNA, ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides is interrupted by non-nucleotide components. Nucleotide or nucleic acid described herein can be modified to comprise modified nucleic acid, nucleic acid analog, modified sugars, sugar analogs, modified nucleic acid linkage, backbone phosphate modification, or a combination thereof.

As used herein, the terms “polypeptide”, “peptide”, and “protein” are used interchangeably to refer to a polymer of amino acid residues. In some cases, a polypeptide refers to a full-length polypeptide as translated from a coding open reading frame, or as processed to its mature form. In some cases, a polypeptide or peptide can be a degradation fragment or a processing fragment of a protein that nonetheless uniquely or identifiably maps to a particular protein. In some cases, a polypeptide can be a single linear polymer chain of amino acids bonded together by peptide bonds between the carboxyl and amino groups of adjacent amino acid residues. A polypeptide can be modified, for example, by the addition of carbohydrate, phosphorylation, etc. As used herein, the terms “fragment” or equivalent terms can refer to a locus of a protein that has less than the full length of the protein and optionally maintains the function of the protein.

“Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN, FASTA, gapped BLAST, BLASTP, BLASTN, or GCG.

The terms “antibody” and “immunoglobulin” are used interchangeably herein and cover fully assembled antibodies, antibody fragments that can bind antigen, for example, Fab, F(ab′)2, Fv, single chain antibodies (scFv), diabodies, antibody chimeras, hybrid antibodies, bispecific antibodies, and the like. In some cases, a binding domain of an antibody is any domain that specifically binds to an antigen, including a binding domain of an antibody or a non-antibody binding domain. In some cases, an antibody binding domain binds to tumor cells, such as an antibody against a tumor cell surface receptor or a tumor antigen. An antibody can be a monoclonal antibody, a polyclonal antibody, a recombinant antibody, or an antigen binding fragment thereof, for example, a heavy chain variable domain (VH) and a light chain variable domain (VL). In some cases, a binding domain of a non-antibody scaffold can be a lipocalin, an anticalin, ‘T-body’, an affibody, a peptibody, a DARPin, an affimer, an avimer, a knottin, a monobody, an affinity clamp, an ectodomain, a receptor ectodomain, a receptor, a cytokine, a ligand, an immunocytokine, a centryin, a T-cell receptor, or a recombinant T-cell receptor. In some cases, a binding domain of an antibody construct is an antigen binding domain from a monoclonal antibody and comprises a light chain and a heavy chain. In some cases, an antibody is a derivatized antibody, such as an antibody modified by glycosylation, acetylation, pegylation, phosphorylation, amidation, derivatization by known protecting/blocking groups, proteolytic cleavage, or the like. An antibody can also be modified, such as by defucosylation or deglycosylation.

The terms “monoclonal antibody” and “mAb” are used interchangeably herein and refer to an antibody obtained from a substantially homogeneous population of antibodies. In some cases, the individual monoclonal antibodies obtained from the substantially homogenous population of antibodies are identical except for naturally occurring mutations that may be present in minor amounts. In some cases, a monoclonal antibody that binds to a tumor antigen comprises a light chain of a tumor antigen antibody and a heavy chain of a tumor antigen antibody. In some cases, the monoclonal antibody binds to an antigen on the surface of an immune cell (immune cell antigen) and comprises a light chain of an anti-immune cell antigen antibody and a heavy chain of an anti-immune cell antigen antibody. In some cases, the monoclonal antibody specifically binds to an antigen present on the surface of an antigen presenting cell (APC antigen) and comprises the light chain of an anti-APC antigen antibody and the heavy chain of an anti-APC antigen antibody, which bind an APC antigen.

The term “antibody fragment” as used herein refers to a molecule that comprises a portion of an intact antibody, preferably the antigen-binding or variable region of an intact antibody. Examples of antibody fragments include Fab, Fab, F(ab′)2, Fv fragments, and single chain fragment variable (scFv); diabodies; linear antibodies; single-chain antibody molecules; and multi-specific antibodies formed from antibody fragments. Digesting antibodies with papain produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions.

The term “human antibody” includes all antibodies that have one or more variable and constant regions derived from human immunoglobulin sequences. In some cases, all of the variable and constant domains of the antibody are derived from human immunoglobulin sequences (referred to as a “fully human antibody”).

The term “recombinant human antibody,” as used herein, is intended to include all human antibodies that are prepared, expressed, created, or isolated by recombinant methods, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies may have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

“Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. In some versions, V regions are humanized by designing consensus sequences of human and mouse V regions, and converting residues outside the CDRs that are different between the consensus sequences.

As used herein, the term “in vivo” is used to describe an event that takes place in a subject's body.

As used herein, the term “ex vivo” is used to describe an event that takes place outside of a subject's body. An “ex vivo” assay cannot be performed on a subject. Rather, it is performed upon a sample separate from a subject. Ex vivo is used to describe an event occurring in an intact cell outside a subject's body.

As used herein, the term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, a “microenvironment” refers to the extracellular environment in which cells targeted by the extracellular vesicles described herein are located. In some cases, the microenvironment can have an extracellular space(s) in which proteases and other soluble proteins and factors are located. A microenvironment can contain, for example, blood vessels, immune cells, fibroblasts, bone marrow-derived inflammatory cells, lymphocytes, signaling molecules and the extracellular matrix (ECM).

“Treating” or “treatment” can refer to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit can refer to eradication of a disorder being treated or amelioration of symptoms of a disorder being treated. Also, a therapeutic benefit may be achieved with the eradication or amelioration of one or more physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For a prophylactic benefit, a subject at risk of developing a particular disease, or a subject reporting one or more of the physiological symptoms of a disease can undergo treatment, even though a diagnosis of a disease has not been made.

The terms “effective amount” and “therapeutically effective amount,” are used interchangeably herein and generally refer to a quantity of a pharmaceutical composition, for example a pharmaceutical composition comprising the composition described herein, that is sufficient to result in a desired activity upon administration to a subject in need thereof. Within the context of the present disclosure, the term “therapeutically effective” refers to that quantity of a pharmaceutical composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component is “pharmaceutically acceptable” in the sense of being compatible with the other ingredients of a pharmaceutical formulation. It can also be suitable for use in contact with the tissue or organ of humans and non-human mammals without excessive toxicity, irritation, allergic response, immunogenicity, or other problems or complications, commensurate with a reasonable benefit/risk ratio.

The term “pharmaceutical composition” refers to the compositions disclosed herein with other chemical components, such as diluents or carriers. The pharmaceutical compositions can facilitate administration to the subject. Multiple techniques of administering a compound exist in the art including, but not limited to, oral, injection, aerosol, parenteral, and topical administration.

The terms “patient” or “subject” are used interchangeably herein and encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

### Overview

The present disclosure relates to the design and production of extracellular vesicles expressing at least one adapter polypeptide (e.g., Fc receptor, CD64) that can be complexed with an antibody. The antibody can direct and target the extracellular vesicle to a cell expressing a cell-surface marker (e.g., an antigen for the antibody) that can be recognized and bound by the antibody. The cell expressing the cell-surface marker can be a diseased cell. In some cases, the cell expressing the cell-surface marker is a cancer cell, a tumor cell, a non-cancerous lesion cell, a cell as part of a damaged tissue, a cell as part of a healthy tissue, or an immune cell.

In some cases, the adapter polypeptide can be further engineered to include an additional targeting domain to enhance the targeting and accumulation of the extracellular vesicle at the targeted cell. This targeting domain can bind to the same or different cell-surface marker expressed by the same targeted cell as that targeted by the antibody complexed to the adapter polypeptide. The extracellular vesicle can be configured to target a different cell (e.g., different cell type, diseased cell) or different cellular marker by complexing the adapter polypeptide with another antibody that targets a different cell-surface marker expressed by the different cell. The extracellular vesicle can be designed to carry a payload such as a therapeutic to be delivered to the targeted cell. The therapeutic delivered by the extracellular vesicle can include a therapeutic polynucleotide, a therapeutic polypeptide, a therapeutic compound, or a cancer drug, or a combination thereof.

This disclosure also provides methods of producing extracellular vesicles comprising the at least one adapter polypeptide and comprising large quantities and qualities of a therapeutic such as a therapeutic polynucleotide (e.g., therapeutic messenger RNA). Some approaches described herein involve transfecting at least one heterologous polynucleotide by nanoelectroporation into a cell, where the at least one heterologous polynucleotide is transcribed and/or translated into at least one adapter polypeptide and/or at least one therapeutic. In some cases, the adapter polypeptide comprises an Fc receptor or a fragment thereof, which can be complexed with a Fc region of an antibody. In some embodiments, the transfected cell is stimulated by the nanoelectroporation to produce and secrete a large number of extracellular vesicles comprising large quantities of the therapeutic (e.g., therapeutic mRNA). The secreted extracellular vesicles can be complexed with any antibody comprising an Fc region, where the complexed antibody targets and directs the extracellular vesicle to a targeted cell expressing a first cell-surface marker that can be recognized and bound by the complexed antibody. The at least one adapter polypeptide can include a targeting domain that binds to a second cell-surface marker expressed by the same targeted cell. As such, the accumulation of the extracellular vesicles comprising the dual targeting domains (e.g., the antibody and targeting domains) at the targeted cell may be increased compared to accumulation of extracellular vesicle without the targeting by the antibody, the targeting domain, or a combination thereof.

### Extracellular Vesicles

Described herein are compositions comprising an extracellular vesicle. Also described herein are methods for producing the extracellular vesicle. In some cases, the extracellular vesicle comprises at least one adapter polypeptide, an antibody complexed with the adapter polypeptide, and at least one therapeutic. In some cases, the adapter polypeptide comprises a targeting domain. In some cases, the at least one adapter polypeptide comprises a peptide sequence that is at least 40%, 50%, 60%, 70%, 80%, 90%, 95%, or 99% identical to a peptide sequence of a cell surface protein. In some cases, the adapter polypeptide comprises a peptide that is at least 70% identical to the CD63 surface protein. In some cases, the adapter polypeptide comprises a peptide that is at least 70% identical to another cell surface protein. In some cases, the adapter polypeptide comprises a peptide that is at least 70% identical to a cell surface protein selected from the group ROR1, PD-L1, EpCAM, EGFR, EGFRIII, EGFRVIII, GPC1, GPC3, DLL3, LICAM, GLAST, and CD138.

In some cases, the at least one adapter polypeptide comprises a peptide sequence that is at least 40%, 50%, 60%, 70%, 80%, 90%, 95%, or 99% identical to a peptide sequence of a Fc binding domain, a Fc receptor, or a fragment thereof that recognizes and binds a Fc region of the antibody. In certain cases, the extracellular vesicle surface protein is the Fc receptor. Fc receptor includes a Fc-gamma receptor, Fc-alpha receptor, or Fc-epsilon receptor. Exemplary Fc receptor includes FcγRI (CD64), FcγRIIa (CD32a), FcγRIIb1(CD32b), FcγRIIb2 (CD32b), FcγRIIc1 (CD32c), FcγRIIc2 (CD32c), FcγRIIc3 (CD32c), FcγRIIc4 (CD32c), FcγRIIc5 (CD32c), FcγRIIIA (CD16a), FcγRIIIB (CD16b), FcεRI, FcεRII (CD23), FcαRI (CD89), Fcα/μR, FcRn, DC-SIGN, or plgR. In some instances, the at least one adapter polypeptide comprises a peptide sequence that is at least 40%, 50%, 60%, 70%, 80%, 90%, 95%, or 99% identical to a peptide sequence of any one of the Fc receptors described herein. In some instances, the at least one adapter polypeptide comprises a peptide sequence that is at least 40%, 50%, 60%, 70%, 80%, 90%, 95%, or 99% identical to a peptide sequence of any one of the Fc receptors: FcγRI (CD64), FcγRII (CD32), or FcγRIII (CD16). In some instances, the at least one adapter polypeptide comprises a peptide sequence that is at least 40%, 50%, 60%, 70%, 80%, 90%, 95%, or 99% identical to a peptide sequence of Fc receptor FcγRI (CD64).

In some cases, the extracellular vesicle described herein comprises at least two adapter polypeptides. In some cases, one of the at least two adapter polypeptides comprises an adapter polypeptide comprising the peptide sequence of a Fc binding domain, a Fc receptor, or a fragment thereof. In some cases, one of the at least two adapter polypeptides comprises the peptide sequence of CD47 or a fragment thereof. In some cases, the extracellular vesicle described herein comprises a first and a second adapter polypeptides, where the first adapter polypeptide comprises the peptide sequence of a Fc binding domain, a Fc receptor, or a fragment thereof and the second adapter polypeptide comprises the peptide sequence of CD47 or a fragment thereof

In some cases, the extracellular vesicle comprises an antibody complexed with the adapter polypeptide comprising a peptide sequence comprising a Fc binding domain, Fc receptor, or a fragment thereof. In some cases, the antibody complexed with the adapter polypeptide binds to a first cell-surface marker of expressed by a targeted cell. In some instances, the adapter polypeptide comprises an extracellular domain. In some cases, a targeting domain is attached to the extracellular domain, where the targeting domain binds to a second cell-surface marker of expressed by the same targeted cell expressing the first cell-surface marker. In some instances, the targeting domain attached to the extracellular domain binds to a second cell-surface marker expressed by the same targeted cell. In some instances, the targeted cell is a cell as part of a healthy tissue or an immune cell. In some instances, the targeted cell is a diseased cell. In some cases, the diseased cell is a cancer cell, a tumor cell, a non-cancerous lesion cell, or a cell as part of a damaged tissue. In some cases, the first and second cell-surface marker expressed by the targeted cell are identical. In some cases, the first and second cell-surface marker expressed by the targeted cell are different.

In some instances, both the antibody and the targeting domain respectively bind the first and second cell-surface marker expressed by the targeted cell. In certain cases, the antibody and the targeting domain bind the first and second cell-surface marker simultaneously. In certain cases, the antibody and the targeting domain bind the first and second cell-surface marker sequentially. In some cases, the antibody is released from being complexed to the adapter polypeptide (i.e. no longer bound to the first cell-surface marker), while the targeting domain remains bound to the second cell-surface marker.

In some cases, the extracellular vesicle described herein is an exosome. In some cases, the exosome comprises an adapter polypeptide comprising a peptide sequence of a Fc binding domain, a Fc receptor, or a fragment thereof. The adapter polypeptide comprising the Fc binding domain, the Fc receptor, or the fragment thereof can be complexed with a Fc region of any one of the antibody described herein. In some cases, the adapter peptide comprises a targeting domain. In some cases, the targeting domain is a tumor homing peptide (THP), a tissue homing peptide, a tissue-targeting domain, a cell-penetrating peptide, a viral membrane protein, or a combination thereof. In some cases, the targeting domain is a tissue homing peptide or a tumor homing peptide. In some cases, the targeting domain is a tumor homing peptide (THP). In some cases, the targeting domain is a tissue homing peptide.

In some cases, the first cell-surface marker can be any one the cell-surface marker (e.g. an antigen or a fragment thereof) described herein that can be recognized and bound by an antibody described herein. In some cases, the second cell-surface marker can be recognized and bound by the targeting domain can be the same or different from the first cell-surface marker. In some cases, the second cell-surface marker can be any macromolecules or proteins expressed on the surface of the cell. In some cases, the second cell-surface marker is expressed by a cell of a specific tissue. In some cases, the second cell-surface marker is expressed by a cancerous cell or by a non-cancerous lesion cell. Non-limiting example of the second cell-surface marker includes vascular receptor, fibronectin receptor, CD44, CD24, ESA, SSEA1, CD133, CD34, CD19, CD38, CD26, CD166, or CD90.

In some instances, the accumulation of the extracellular vesicle comprising the antibody complexed with the at least one adapter polypeptide at the cell expressing the first and second cell-surface marker is higher than accumulation of extracellular vesicle without the antibody complexed with the at least one adapter polypeptide at the cell expressing the first and second cell-surface marker. In some instances, the accumulation of the extracellular vesicle comprising the antibody complexed with the at least one adapter polypeptide at the cell expressing the first and second cell-surface marker is at least 2 fold, 5 fold, 10 fold, 50 fold, 100 fold, or higher compared to the accumulation of extracellular vesicle without the antibody complexed with the at least one adapter polypeptide at the cell expressing the first and second cell-surface marker. In certain cases, the accumulation of the extracellular vesicle comprising the antibody complexed with the at least one adapter polypeptide at the cell expressing the first and second cell-surface marker is higher than accumulation of extracellular vesicle without the at least one adapter polypeptide at the cell expressing the first and second cell-surface marker. In some instances, the accumulation of the extracellular vesicle comprising the antibody complexed with the at least one adapter polypeptide at the cell expressing the first and second cell-surface marker is at least 2 fold, 5 fold, 10 fold, 50 fold, 100 fold, or higher compared to the accumulation of extracellular vesicle without the at least one adapter polypeptide at the cell expressing the first and second cell-surface marker.

The antibody complexed with the adapter polypeptide comprises any one of the antibody fragment or the binding domain of an antibody described herein. In some cases, the antibody is fused to a Fc region, where the Fc region is recognized by the adapter polypeptide. In some instances, the Fc region comprises IgA, IgD, IgE, IgG, or IgM. In some cases, the Fc region comprises IgA, IgD, or IgG. In some instances, the Fc region comprises IgG. IgG can be IgG1, IgG2, IgG3, or IgG4. In some instances, the Fc region comprises IgG1 or IgG3. In some cases, the antibody described herein comprises a Fc region comprising IgG1 or IgG3 to be complexed with the adapter polypeptide described herein. In some cases, the antibody is complexed to the adapter polypeptide via non-covalent complexing of the adapter polypeptide to the Fc region of the antibody. In some cases, the antibody is a monoclonal antibody. In some instances, the antibody is a humanized antibody. In some cases, the antibody is a humanized monoclonal antibody.

In some cases, the extracellular vesicle described herein comprises at least one adapter polypeptide. In some instances, the adapter polypeptide comprises at least one targeting domain attached to the extracellular domain of the adapter polypeptide. In some cases, the at least one targeting domain is a tumor homing peptide (THP), a tissue-targeting domain, a cell-penetrating peptide, a viral membrane protein, or a combination thereof. In some cases, the tissue-targeting domain is a tissue homing peptide.

In some instances, the at least one targeting domain is the tumor homing peptide, where the tumor homing peptide targets a cancerous cell. In some instances, the at least one targeting domain is the tumor homing peptide, where the tumor homing peptide targets a cell as part of a tumor (such as a spheroid tumor). In some instances, the at least one targeting domain is the tumor homing peptide, where the tumor homing peptide targets a non-cancerous lesion cell.

In some cases, the adapter polypeptide comprises at least one, two, three, four, five, or more targeting domains. In some instances, the at least two targeting domains can be identical. In some cases, the at least two targeting domains can be different. The targeting domain can be complexed to the N-terminus of the adapter polypeptide. In an alternative, the targeting domain can be complexed to the C-terminus of the adapter polypeptide. In some instances, the targeting domain can be integrated into the adapter polypeptide. In some cases, the targeting domain is complexed to the adapter polypeptide via a peptide linker. In some cases, the linker peptide comprises 5 to 200 amino acids. In other cases, the linker peptide comprises 5 to 25 amino acids. In some instances, the linker peptide can be rigid (e.g. (EAAAK)₁₋₃, A(EAAAK)₄ALEA(EAAAK)₄A, PAPAP, AEAAAKEAAAKA, or (AP)₁₀₋₃₄,), flexible (e.g. (GGGGS)₁₋₄ or (Gly)₆₋₈), or cleavable (e.g. VSQTSKLTR↓AETVFPDV, PLG↓LWA, RVL↓AEA, EDVVCC↓SMSY, GGIEGR↓GS, TRHRQPR↓GWE, AGNRVRR↓SVG, RRRRRRR↓R↓R, or GFLG↓). In some cases, the linker peptide is a FLAG linker comprising a peptide sequence of DYKDDDDK. In certain cases, the FLAG linker can be a 3×FLAG linker comprising a peptide sequence of YKDHD-G-DYKDHD-I-DYKDDDDK.

In some cases, the adapter polypeptide comprises at least one tumor homing peptide. In some cases, the adapter polypeptide comprises at least two, three, four, five, or more tumor homing peptides. In some instances, the at least two tumor homing peptides are identical. In some cases, the at least two tumor homing peptides are different. In some cases, the tumor homing peptide is fused to an N-terminus of the adapter polypeptide. In some cases, the tumor homing peptide is fused to an C-terminus of the adapter polypeptide. In some cases, the tumor homing peptide can be integrated at any peptide location of the adapter polypeptide. In some instances, the tumor homing peptide comprises at least 3, 4, 5, 6, 7, 8, 9, 10, 15, 20, 25, 30, 40, 50, or 100 amino acids. In some cases, the tumor homing peptide is a CDX (FKESWREARGTRIERG) peptide. In some cases, the tumor homing peptide is a CREKA peptide. In some cases, the tumor homing peptide is a CKAAKN peptide. In some cases, the tumor homing peptide is a ARRPKLD peptide. Other exemplary tumor homing peptide can include those that target lung cancer (including SVSVGMKPSPRP, PRPSPKMGVSVS, TDSILRSYDWTY, CSNIDARAC, and ARRPKLD), gastric cancer (including CGNSNPKSC, GRRTRSRRLRRS, CTKNSYLMC, and AADNAKTKSFPV), pancreatic cancer (including CRGRRST, CRSRKG, and CKAAKN), prostate cancer (including FRPNRAQDYNTN, IAGLATPGWSHWLAL, CREAGRKAC, and CAGRRSAYC), squamous carcinoma (including CSRPRRSEC, CGKRK, and CDTRL), melanoma (including TAASGVRSMH, LTLRWVGLMS, CVNHPAFAC, and CLSDGKRKC), hepatocellular carcinoma (including KSLSRHDHIHHH, and SFSIIHTPILPL), colon cancer (including CPHSKPCLC, CPIEDRPMC, CTPSPFSHC, and VHLGYAT), bladder cancer (including CSNRDARRC and CQDGRMGFC), breast cancer (including CREKA), glioma (including LWATFPPRPPWL and LLADTTHHRPWT), ovarian cancer (including CDGLGDDC, CDGWGPNC, and RLLDTNRPLLPY), and head and neck cancer (including TSPLNIHNGQKL and SPRGDLAVLGHKY).

In some cases, the adapter polypeptide comprises at least one tissue-targeting domain, which targets and directs the extracellular vesicle comprising the adapter polypeptide to a specific tissue. In some cases, the tissue-targeting domain is the tissue homing domain. In some cases, the adapter polypeptide comprises at least two, three, four, five, or more tissue-targeting peptides. In some instances, the at least two tissue-targeting peptides are identical. In some cases, the at least two tissue-targeting peptides are different. In some cases, tissue-targeting peptide is fused to an N-terminus of the adapter polypeptide. In some cases, the tissue-targeting peptide is fused to a C-terminus of the adapter polypeptide. In some cases, the tissue-targeting peptide can be integrated at any peptide location of the adapter polypeptide. In some instances, the tissue-targeting peptide comprises at least 3, 4, 5, 6, 7, 8, 9, 10, 15, 20, 25, 30, 40, 50, or 100 amino acids. Exemplary tissue-targeting domain which targets endothelial or cardiac tissue includes SIGYPLP, LSIPPKA, FQTPPQL, LTPATAI, CNIWGVVLSWIGVFPEC, NTTTH, VHPKQHR(tetramer), CRKRLDRNCCRTLTVRKC, CLWTVGGGC, QPWLEQAYYSTF, YPHIDSLGHWRR, LLADTTHHRPWT, SAHGTSTGVPWP, VPWMEPAYQRFL, TLPWLEESYWRP, HWRR, CSTSMLKAC, DDTRHWG, CARPAR, CKRAVR, CRSTRANPC, CPKTRRVPC, CSGMARTKC, or CRPPR. Exemplary tissue-targeting domain which targets pancreatic tissue includes CRVASVLPC, SWCEPGWCR, LSGTPERSGQAVKVKLKAIP, CHVLWSTRCCVSNPRWKC, or LSALPRT. Exemplary tissue-targeting domain which targets kidney tissue includes CLPVASC, ELRGD(R/M)AX(W/L), GV(K/R)GX3(T/S)RDXR, HITSLLSHTTHREP, or ANTPCGPYTHDCPVKR. Exemplary tissue-targeting domain which targets lung tissue includes CGFELETCCGFECVRQCPERC, QPFMQCLCLIYDASCRNVPPIFNDVYWIAF, VNTANST, CTSGTHPRC, or SGEWVIKEARGWKHW-VFYSCCPTTPYLDITYH. Exemplary tissue-targeting domain which targets intestinal tissue includes YSGKWGW, LETTCASLCYPSYQCSYTMPHPPVVPPHPMTYSCQY, YPRLLTP, CSQSHPRHC, CSKSSDYQC, CKSTHPLSC, CTGKSCLRVG, SFKPSGLPAQSL, or CTANSSAQC. Exemplary tissue-targeting domain which targets brain tissue can include CLSSRLDAC, GHKAKGPRK, HAIYPRH, THRPPMWSPVWP, HLNILSTLWKYRC, CAGALCY, CLEVSRKNC, RPRTRLHTHRNR(D-aa), ACTTPHAWLCG, GLAHSFSDFARDFV, GYRPVHNIRGHWAPG, TGNYKALHPHNG, CRTIGPSVC, CTSTSAPYC, CSYTSSTMC, CMPRLRGC, TPSYDTYAAELR, RLSSVDSDLSGC, CAQK, or SGVYKVAYDWQH. Additional exemplary tissue-targeting domain targeting various tissue includes LMLPRAD (targeting adrenal gland), CSCFRDVCC (targeting retina), CRDVVSVIC (targeting retina), CVALCREACGEGC (targeting skin hypodermal vasculature), GLSGGRS (targeting uterus), WYRGRL (targeting cartilage), CPGPEGAGC (targeting breast vasculature), SMSIARLVSFLEYR (targeting prostate), GPEDTSRAPENQQKTGC (targeting skin Langerhans), CKGGRAKDC (targeting white fat vasculature), CARSKNKDC (targeting wound or damaged tissue), CHAQGSAEC (targeting thymus), LEPRWGFGWWLKLSTHTTESRSMV (targeting ear or cochlea tissue), ACSTEALRHCGGGS (targeting retinal vessel) or ASSLNIA (targeting muscle tissue).

In some cases, the adapter polypeptide comprises at least two, three, four, five, or more cell-penetrating peptides. In some cases, the adapter polypeptide comprising the cell-penetrating peptide increases the rate of the extracellular vesicle being fused or endocytosed by the targeted cell. In some instances, the at least two cell-penetrating peptides are identical. In some cases, the at least two cell-penetrating peptides are different. In some cases, the cell-penetrating peptide is fused to an N-terminus of the adapter polypeptide. In some cases, the cell-penetrating peptide is fused to an C-terminus of the adapter polypeptide. In some cases, the cell-penetrating peptide can be integrated at any peptide location of the adapter polypeptide. In some instances, the cell-penetrating peptide comprises at least 3, 4, 5, 6, 7, 8, 9, 10, 15, 20, 25, 30, 40, 50, or 100 amino acids. Non-limiting example of the cell-penetrating peptide includes DSLKSYWYLQKFSWR, DWLKAFYDKVAEKLKEAF, KSKTEYYNAWAVWERNAP, GNGEQREMAVSRLRDCLDRQA, HTPGNSNKWKHLQENKKGRPRR, DWLKAFYDKVAEKLKEAF, R9GPLGLAGE8, Ac-GAFSWGSLWSGIKNFGSTVKNYG, RLRWR, LGQQQPFPPQQPY, ILGKLLSTAAGLLSNL, TFFYGGSRGKRNNFKTEEY, Ac-LRKLRKRLLRX-Bpg-G, Ac-LRKLRKRLLR, or MVRRFLVTLRIRRACGPPRVRV.

In some cases, the adapter polypeptide comprises at least two, three, four, five, or more viral membrane proteins or fragments thereof. In some cases, the adapter polypeptide comprising the viral membrane protein increases the rate of the extracellular vesicle being fused or endocytosed by the targeted cell. In some instances, the at least two viral membrane proteins are identical. In some cases, the at least two viral membrane proteins are different. In some cases, the viral membrane protein is fused to an N-terminus of the adapter polypeptide. In some cases, the viral membrane protein is fused to a C-terminus of the adapter polypeptide. In some cases, the viral membrane protein can be integrated at any peptide location of the adapter polypeptide. In some instances, the viral membrane protein comprises at least 3, 4, 5, 6, 7, 8, 9, 10, 15, 20, 25, 30, 40, 50, or 100 amino acids. Non-limiting example of the viral membrane protein includes hemagglutinin, glycoprotein 41, envelop protein, VSV G, HSV01 gB, ebolavirus glycoprotein, or fusion-associated small transmembrane (FAST) protein.

In some cases, the extracellular vesicle described herein comprises at least one therapeutic. In some cases, the therapeutic is a therapeutic polynucleotide. In some cases, the therapeutic is a therapeutic polypeptide. In some instances, the therapeutic is a therapeutic compound. In some cases, the therapeutic is a cancer drug comprising therapeutic polynucleotide, therapeutic polypeptide, therapeutic compound, or a combination thereof. In some instances, the extracellular vesicle comprises a plurality of therapeutics, where the plurality of therapeutics comprises therapeutic polynucleotide, therapeutic polypeptide, therapeutic compound, or a combination thereof.

In some cases, the extracellular vesicle comprises the at least one therapeutic, where the at least one therapeutic is expressed on an extracellular surface of the extracellular vesicle. In some cases, the at least one therapeutic is expressed on the surface of the extracellular vesicle by attaching the at least one therapeutic to the adapter polypeptide. In some instances, the at least one therapeutic is expressed and inserted into the membrane of the extracellular vesicle. In some cases, the at least one therapeutic is within the extracellular vesicle.

In some cases, the extracellular vesicle can be any membrane-bound particle. In some cases, the extracellular vesicle can be any membrane-bound particle secreted by a cell. In some instances, the extracellular vesicle can be any membrane-bound particle produced in vitro. In some instances, the extracellular vesicle can be any membrane-bound particle produced without a cell. In some cases, the extracellular vesicle can be an exosome, a microvesicle, a retrovirus-like particle, an apoptotic body, an apoptosome, an oncosome, an exopher, an enveloped virus, an exomere, or other very large extracellular vesicle. In some cases, the extracellular vesicle is an exosome.

In some cases, the extracellular vesicle can have a diameter about 10 nm to about 10,000 nm. In some cases, the extracellular vesicle can have a diameter about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 50 nm to about 100 nm, about 50 nm to about 500 nm, about 50 nm to about 1,000 nm, about 50 nm to about 5,000 nm, about 50 nm to about 10,000 nm, about 100 nm to about 500 nm, about 100 nm to about 1,000 nm, about 100 nm to about 5,000 nm, about 100 nm to about 10,000 nm, about 500 nm to about 1,000 nm, about 500 nm to about 5,000 nm, about 500 nm to about 10,000 nm, about 1,000 nm to about 5,000 nm, about 1,000 nm to about 10,000 nm, or about 5,000 nm to about 10,000 nm. In some cases, the extracellular vesicle can have a diameter about 10 nm, about 50 nm, about 100 nm, about 500 nm, about 1,000 nm, about 5,000 nm, or about 10,000 nm. In some cases, the extracellular vesicle can have a diameter at least about 10 nm, about 50 nm, about 100 nm, about 500 nm, about 1,000 nm, or about 5,000 nm. In some cases, the extracellular vesicle can have a diameter at most about 50 nm, about 100 nm, about 500 nm, about 1,000 nm, about 5,000 nm, or about 10,000 nm.

### Antibodies

Described herein are compositions comprising an extracellular vesicle comprising at least one adapter polypeptide complexed with an antibody. In some cases, the antibody is complexed to the adapter polypeptide via non-covalent complexing between the adapter polypeptide to the Fc region of the antibody. In some cases, the antibody is a monoclonal antibody. In some instances, the antibody is a humanized antibody. In some cases, the antibody is a humanized monoclonal antibody.

In some instances, upon complexing with the at least one adapter polypeptide, the antibody directs the extracellular vesicle to a cell by binding to a cell-surface marker expressed by the cell. In some cases, the antibody directs the extracellular vesicle to a diseased cell by binding to a cell-surface marker expressed by the diseased cell. In some cases, the diseased cell is a cancer cell. In some instances, the diseased cell is a non-cancerous lesion cell. In some cases, the diseased cell is a tumor cell. In some instances, the cell-surface marker is an antigen associated with a cancer cell or a non-cancerous lesion cell. Exemplary cell-surface marker associated with the cancer cell or the non-cancerous lesion cell that can be recognized and bound by the antibody described herein includes 1-40-β-amyloid, 4-1BB (CD137), 5AC, 5′-nucleotidase, 5T4, activated F9, F10, activin receptor-like kinase 1, ACVR2B, adenocarcinoma antigen, alpha-fetoprotein, amyloid, angiopoietin 2, angiopoietin 3, anthrax toxin, protective antigen, AOC3 (VAP-1), AXL, B7-H3, Bacillus anthracis anthrax, BAFF, BAFF-R, BCMA, beta amyloid, B-lymphoma cell, Cls, C242 antigen, C5, CA-125, CA-125 (imitation), calcitonin, calcitonin gene-related peptide, calcitonin gene-related peptide alpha, Canis lupus familiaris IL31, carbonic anhydrase 9 (CA-IX), cardiac myosin, CCL11 (eotaxin-1), CCR2, CCR4, CCR5, CD11, CD18, CD123, CD125, CD134, CD147 (basigin), CD15, CD152, CD154 (CD40L), CD19, CD19, CD3E, CD2, CD20, CD200, CD22, CD23 (IgE receptor), CD25 (α chain of IL-2 receptor), CD27, CD276, CD278, aka ICOS, CD28, CD3, CD3 epsilon, CD30 (TNFRSF8), CD319, CD33, CD37, CD38, CD3E, CD4, CD40, CD41 (integrin alpha-IIb), CD44 v6, CD45, CD5, CD51, CD52, CD56, CD6, CD70, CD74, CD79B, CD80, CD97B, CEA, CEACAM5, CEA-related antigen, CFD, CGRP, Claudin 18 Isoform 2, CLDN18.2, Clostridium difficile, clumping factor A, c-Met, coagulation factor III, complement C5a, MCSF, CSF1, CSF1R, CSF2, CTGF, CTLA-4, CXCR4 (CD184), cytomegalovirus, cytomegalovirus glycoprotein B, dabigatran, dendritic cell-associated lectin 2, DLL3, DLL4, DPP4, DR5, E. coli shiga toxin type-1, E. coli shiga toxin type-2, ebolavirus glycoprotein, EGFL7, EGFR, EGFR extracellular domain III, EGFR, cMet, EGFR, HER1, EGRF, ERBB1 HER1, endoglin, endotoxin, EpCAM, EPHA3, ephrin receptor A3, episialin, ERBB3 (HER3), ERBB3, HER3, Escherichia coli, F protein of respiratory syncytial virus, FAP, FCGRT, FGF 23, FGFR2, fibrin II, beta chain, fibronectin extra domain-B, folate hydrolase, folate receptor 1, folate receptor alpha, Frizzled receptor, GCGR, GD2 ganglioside, GDF-8, gelatinase B, glypican 3, GMCSF, GMCSF receptor α-chain, GPNMB, GPRC5D, CD3, growth differentiation factor 8, GUCY2C, hemagglutinin, hepatitis B surface antigen, hepatitis B virus, HER1, HER2, HER2/neu, HER2/neu, CD3, HGF, HGFR, HHGFR, histone complex, HIV-1, HLA-DR ?, HNGF, Hsp90, human scatter factor receptor kinase, human TNF, human beta-amyloid, ICAM-1, ICOSL, IFN-α, IFN-γ, IgE, IgE Fc region, IGF-1 receptor (CD221), IGF1, IGF2, IGF1R, CD221, IGHE, IL 17A, IL 17A and IL 17F, IL 20, IL 3 receptor, IL-1, IL-12, IL-13, IL-17, IL17A and IL 17F, ILIA, IL-1B, IL2, IL-22, IL23, IL23A, IL31RA, IL-4, IL-4Rα, IL-5, IL-6, IL6 receptor, IL-6 receptor, IL6R, IL-6R, IL9, ILGF2, influenza A virus hemagglutinin, integrin α4β7, integrin α4, integrin α4β7, integrin α5β1, integrin αIIbβ3, integrin αvβ3, integrin β7, interferon gamma, interferon receptor, interferon α/β receptor, interferon gamma-induced protein, interleukin 1 alpha, interleukin 13, interleukin 17 alpha, interleukin 17 alpha, TNF, interleukin 17A, ITGA2 (CD49b), ITGB2 (CD18), kallikrein, KIR2D, LAG3, Lewis-Y antigen, LFA-1 (CD11a), LINGO-1, lipoteichoic acid, LIV-1, LOXL2, LRRC15, L-selectin (CD62L), LTA, LYPD3, MASP-2, MCAM, MCP-1, mesothelin, MIF, MS4A1, MSLN, MST1R (aka RON), MUC1, mucin CanAg, mucosal addressin cell adhesion molecule, myelin-associated glycoprotein, myostatin, NACP, NCA-90 (granulocyte antigen), nectin-4, neural apoptosis-regulated proteinase 1, NGF, NGNA ganglioside, NKG2A, NOGO-A, Notch 1, Notch receptor, NRP1, OX-40, oxLDL, PCDC1, PCSK9, PD-1, PDCD1, PDCD1, CD279, PDGF-Rα, PDGFRA, PD-L1, phosphate-sodium co-transporter, phosphatidylserine, platelet-derived growth factor receptor beta, prostatic carcinoma cells, Pseudomonas aeruginosa, Pseudomonas aeruginosa type III secretion system, PTK7, rabies virus G glycoprotein, rabies virus glycoprotein, RANKL, respiratory syncytial virus, RGMA, RHD, Rhesus factor, root plate-specific spondin 3, ROR1, RSV fusion glycoprotein, RSVFR, RTN4, sclerostin, SDC1, selectin P, serum amyloid A protein, serum amyloid P component, SLAMF7, SLITRK6, SOST, sphingosine-1-phosphate, Staphylococcus aureus, Staphylococcus aureus alpha toxin, Staphylococcus aureus bi-component leukocidin, STEAP1, TAG-72, tau protein, T-cell receptor, TEM1, tenascin C, TFPI, TGF beta 1, TGF beta 2, TGF-β, TIGIT, TNFR superfamily member 4, TNF-α, TRAIL-R1, TRAIL-R2, TRAP, TROP-2, TSLP, tumor antigen CTAA16.88, tumor specific glycosylation of MUC1, TWEAK receptor, TYRPI (glycoprotein 75), VEGF-A, VEGF-A and Ang-2, VEGFR-1, VEGFR2, vimentin, VSIR, VWF, or Zaire ebolavirus glycoprotein. In some instances, the cell-surface marker that is recognized and bound by the antibody described herein comprises EGFR, PD-L1, or ROR1.

In some instances, the antibody complexed with the at least one adapter polypeptide comprises any one of the antibody fragments or binding domains of the antibodies described herein. In some cases, the antibody complexed with the at least one adapter polypeptide comprises a Fc region comprising a peptide sequence that binds one or more Fc binding domains or Fc receptors or fragments thereof described herein. The Fc region can be from an antibody. The Fc region of an antibody can be selected from the classes of immunoglobins, IgA, IgD, IgE, IgG, or IgM. Several different classes can be further divided into isotypes such as IgG1, IgG2, IgG3, IgG4, IgA1, and IgA2. The heavy-chain constant regions of Fc that correspond to the different classes of immunoglobulins can be α, δ, ε, γ, and μ, respectively. The light chains can be one of either kappa or K and lambda or 2. The Fc region comprises a number of Fc domains, CH1, CH2, CH3 and CH4, according to the type of antibody.

Any antibody comprising the Fc region comprising immunoglobin IgA, IgD, IgE, IgG, or IgM can be complexed with the adapter polypeptide comprising the Fc binding domain, Fc receptor, or a fragment thereof. In some cases, the Fc region has an IgG1 isotype. In some cases, the Fc region has an IgG2 isotype. In some cases, the Fc region has an IgG3 isotype. In some cases, the Fc region has an IgG4 isotype. In some cases, the Fc region has a hybrid isotype comprising constant regions from two or more isotypes. Antibody to be complexed with the adapter polypeptide can include any antibody or antigen binding fragment comprising an Fc domain. Exemplary antibody to be complexed with the adapter polypeptide described herein comprises Cetuximab (including clone C225), Atezolizumab, anti-PD-L1 (including clone SP142) or anti-ROR1 mAb (including clone 2A2).

In some cases, the Fc region comprises a peptide sequence having a K_{d} for the at least one adapter polypeptide comprising any one of the Fc binding domain, the Fc receptor, or the fragment thereof described herein. In some cases, the Fc region comprises a peptide sequence having a K_{d} for the at least one adapter polypeptide comprising the Fc receptor or the fragment thereof. In some cases, the Fc receptor comprises FcγRI (CD64), FcγRIIa (CD32a), FcγRIIb1(CD32b), FcγRIIb2 (CD32b), FcγRIIc1 (CD32c), FcγRIIc2 (CD32c), FcγRIIc3 (CD32c), FcγRIIc4 (CD32c), FcγRIIc5 (CD32c), FcγRIIIA (CD16a), FcγRIIIB (CD16b), FcεRI, FcεRII (CD23), FcαRI (CD89), Fcα/μR, FcRn, DC-SIGN, or plgR. In some cases, the Fc region comprises a peptide sequence having a K_{d} for the at least one adapter polypeptide comprising FcγRI (CD64), FcγRIIa (CD32a), FcγRIIb1(CD32b), FcγRIIb2 (CD32b), FcγRIIc1 (CD32c), FcγRIIc2 (CD32c), FcγRIIc3 (CD32c), FcγRIIc4 (CD32c), FcγRIIc5 (CD32c), FcγRIIIA (CD16a), or FcγRIIIB (CD16b). In some cases, the Fc region comprises a peptide sequence having a K_{d} for the at least one adapter polypeptide comprising FcγRI (CD64).

In some instance, the Fc region has a peptide sequence that is modified, as compared to a wild type Fc sequence, to alter at least one constant region-mediated biological effector function relative to the corresponding antibody comprising the wild type Fc region. For example, an Fc region can be modified by amino acid substitution to decrease or increase at least one Fc-mediated binding to an Fc receptor as determined to the change in dissociation constant (K_{d}).

In some cases, any one of the antibody described herein can comprise a Fc region that is modified to increase at least one Fc region-mediated biological effector function relative to an antibody comprising an unmodified Fc domain. For example, an antibody described herein with a modified Fc region that binds to any one of the Fc binding domain or Fc receptor described herein with increased affinity than the corresponding antibody comprising the wild type Fc region. Such modified Fc region can be produced according to the methods known to a skilled artisan. In some instances, the Fc region described herein comprises a peptide sequence having at least one, two, three, four, five, six, seven, eight, nine, ten or more amino acid modifications.

In some instances, the Fc region comprising the at least one amino acid modification exhibits decreased K_{d} of binding to any one of the Fc binding domain or Fc receptor described herein relative to the K_{d} of binding between wild type Fc to the same Fc binding domain or Fc receptor. In some instances, the Fc region comprising the at least one amino acid modification exhibits decreased K_{d} to any one of the Fc binding domain or Fc receptor described herein relative to the K_{d} of binding between wild type Fc to the same Fc binding domain or Fc receptor across a pH range of 6.5 to 8.4. In some instances, the Fc region comprising the at least one amino acid modification is configured to be complexed to the adapter polypeptide comprising the Fc receptor at an acidic pH or acidic microenvironment.

In some instances, the Fc region comprising the at least one amino acid modification exhibits increased K_{d} of binding to any one of the Fc binding domain or Fc receptor described herein relative to the K_{d} of binding between wild type Fc to the same Fc binding domain or Fc receptor. In some instances, the Fc region comprising the at least one amino acid modification exhibits increased K_{d} to any one of the Fc binding domain or Fc receptor described herein relative to the K_{d} of binding between wild type Fc to the same Fc binding domain or Fc receptor across a pH range of 6.5 to 8.4. In some instances, the Fc region comprising the at least one amino acid modification is configured to be released from being complexed to the adapter polypeptide comprising the Fc receptor at an acidic pH or acidic microenvironment.

### Fc Binding

In some instances, the adapter polypeptide comprising the Fc binding domain or the Fc receptor can be complexed with the Fc region of any one of the antibody described herein. In some cases, the Fc binding domain is a fragment of the Fc receptor that binds to the Fc region of the antibody. Exemplary Fc receptor includes FcγRI (CD64), FcγRIIa (CD32a), FcγRIIb1(CD32b), FcγRIIb2 (CD32b), FcγRIIc1 (CD32c), FcγRIIc2 (CD32c), FcγRIIc3 (CD32c), FcγRIIc4 (CD32c), FcγRIIc5 (CD32c), FcγRIIIA (CD16a), FcγRIIIB (CD16b), FcεRI, FcεRII (CD23), FcαRI (CD89), Fcα/μR, FcRn, DC-SIGN, or plgR. In some instances, the at least one adapter polypeptide comprises a peptide sequence that is at least 40%, 50%, 60%, 70%, 80%, 90%, 95%, or 99% identical to a peptide sequence of any one of the Fc receptor: FcγRI (CD64), FcγRII (CD32), or FcγRIII (CD16). In some instances, the at least one adapter polypeptide comprises a peptide sequence that is at least 40%, 50%, 60%, 70%, 80%, 90%, 95%, or 99% identical to a peptide sequence of Fc receptor FcγRI (CD64).

In some cases, the adapter polypeptide comprises a Fc binding domain comprising bacterial protein that binds to the Fc region of the antibody. In some cases, the Fc binding domain comprises Protein A, Protein G, Protein L, Protein Z, Protein LG, Protein LA, Protein AG, or a fragment thereof. In some cases, the Fc binding domain comprises peptide or peptidomimetics. Exemplary Fc binding peptide sequence or peptidomimetic includes TWKTSRISIF, FGRLVSSIRY, EPIHRSTLTALL HWRGWV, HYFKFD, HFRRHL, HWCitGWV, D2AAG, DAAG, cyclo[(Nα-Ac)S(A)-RWHYFK-Lact-E], cyclo[(Nα-Ac)-Dap(A)-RWHYFK-Lact-E], cyclo[Link-M-WFRHYK], NKFRGKYK, NARKFYKG, FYWHCLDE(1), FYCHWALE(2), FYCHTIDE, RRGW, KHRFNKD, APAR, PAM, Fc-111, FcBP-1, FcBP-2, Fc-III-4C, FcRM,

### Therapeutic Cargo

Described herein, in some cases, are extracellular vesicles comprising at least one therapeutic. In some instances, the at least one therapeutic is a therapeutic polynucleotide encoded by the at least one heterologous polynucleotide or vector (e.g., plasmid) transfected into the cell that produces and secrete the extracellular vesicles. In some cases, the at least one therapeutic polynucleotide comprises a nucleic acid sequence that can be translated into a therapeutic polypeptide by the cell targeted and bound by the adapter polypeptide described herein.

In some cases, the extracellular vesicle comprises at least one therapeutic polynucleotide. In some cases, the extracellular vesicle comprises at least 1, 2, 5, 10, 50, 100, 500, 1,000, or more copies of the therapeutic polynucleotide. In some instances, the extracellular vesicle comprises at least two therapeutic polynucleotides. In some instances, the extracellular vesicle comprises at least two therapeutic polynucleotides, where the at least two therapeutic polynucleotides are different. In some cases, the at least two different therapeutic polynucleotides encapsulated by the extracellular vesicle comprise different ratios. For example, the ratio between the first and the second of the two different therapeutic polynucleotides can be 1:1,000, 1:500, 1:100, 1:50, 1:10, 1:5, 1:4, 1:3, 1:2, or 1:1.

In some cases, the therapeutic polynucleotide comprises mRNA, tRNA, SRP RNA, tRNA, tmRNA, snRNA, snoRNA, gRNA, aRNA, crRNA, lncRNA, miRNA, ncRNA, piRNA, siRNA, shRNA, or a combination thereof. In some cases, the therapeutic polynucleotide comprises mRNA. In some cases, the mRNA is intact, i.e. encoding a full length of a protein. In some cases, the mRNA encodes a portion of the protein. In some cases, the mRNA comprises at least 100, 200, 500, 1,000, 5,000, or more of RNA nucleotides. In some instances, therapeutic polynucleotide comprises DNA. In some instances, therapeutic polynucleotide comprises DNA such as vectors (e.g., plasmids) that encode therapeutic polypeptide.

In some instances, a copy number of the therapeutic polynucleotide encapsulated in the extracellular vesicle is at least 1, 2, 3, 5, 10, 100, or more copies of the therapeutic polynucleotide. In some instances, a copy number of the therapeutic polynucleotide comprising RNA encapsulated in the extracellular vesicle is at least 1, 2, 3, 5, 10, 100, or more copies of the therapeutic polynucleotide. In some instances, a copy number of the therapeutic polynucleotide comprising therapeutic messenger RNA encapsulated in the extracellular vesicle is least 1, 2, 3, 5, 10, 100, or more copies of the therapeutic messenger RNA.

In some instances, a copy number of the therapeutic polynucleotide (e.g. RNA therapeutic) encapsulated in the extracellular vesicle produced from cell transfected by microchannel electroporating or nanochannel electroporating is increased compared to a copy number of the therapeutic polynucleotide encapsulated in the extracellular vesicle produced from cell transfected by other methods of transfection by at least 0.1 fold, 0.2 fold, 0.5 fold, 2 fold, 5 fold, 10 fold, 50 fold, 100 fold, 500 fold, 1,000 fold, 5,000 fold, 10,000 fold, or more. In some instances, a copy number of the therapeutic polynucleotide (e.g. RNA therapeutic) encapsulated in the extracellular vesicle produced from cell transfected by microchannel electroporating or nanochannel electroporating is increased compared to a copy number of the therapeutic polynucleotide encapsulated in the extracellular vesicle by introducing the therapeutic polynucleotide directly into the extracellular vesicle (i.e. directly transfecting the therapeutic polynucleotide into the extracellular vesicle) by at least 0.1 fold, 0.2 fold, 0.5 fold, 2 fold, 5 fold, 10 fold, 50 fold, 100 fold, 500 fold, 1,000 fold, 5,000 fold, 10,000 fold, or more.

In some instances, a therapeutic polynucleotide (e.g., an RNA therapeutic) encapsulated within an extracellular vesicle produced from a cell transfected by microchannel electroporation or nanochannel electroporation as described herein is more intact compared to a therapeutic polynucleotide encapsulated in an extracellular vesicle produced from a cell transfected by other methods of transfection. For example, more copies of the RNA therapeutic encapsulated in the extracellular vesicle produced from cell transfected by microchannel electroporating or nanochannel electroporating are intact or full length messenger RNA that can be translated into therapeutic polynucleotide by the targeted cell. In some cases, the copy number of intact RNA therapeutic encapsulated in the extracellular vesicle produced from cell transfected by microchannel electroporating or nanochannel electroporating is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to copy number of intact RNA therapeutic encapsulated in the extracellular vesicle produced from cell transfected by other methods of transfection. In some cases, the copy number of intact RNA therapeutic encapsulated in the extracellular vesicle produced from cell transfected by microchannel electroporating or nanochannel electroporating is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to copy number of intact RNA therapeutic encapsulated in the extracellular vesicle produced from introducing the therapeutic polynucleotide directly into the extracellular vesicle (i.e. directly transfecting the therapeutic polynucleotide into the extracellular vesicle).

In some instances, the extracellular vesicle described herein comprises at least one therapeutic polypeptide. In some cases, the at least one therapeutic polypeptide is encoded by the at least one heterologous polynucleotide or vector (e.g., plasmid) transfected into an extracellular vesicle donor cell.

In some cases, the therapeutic polynucleotides can be translated by the extracellular vesicle donor cells to obtain at least one therapeutic polypeptide. In some cases, the therapeutic polypeptide is attached to the adapter polypeptide described herein. In some cases, the therapeutic polypeptide is inserted into the membrane of the extracellular vesicle. In some cases, the therapeutic polypeptides encoded by the therapeutic polynucleotides can be encapsulated by the extracellular vesicles produced and secreted by the extracellular vesicle donor cells. In some cases, the extracellular vesicles can encapsulate both therapeutic polynucleotides and therapeutic polypeptides encoded by the nanoelectroporated vectors (e.g., plasmids). In some cases, the extracellular vesicles can be exosomes.

In some instances, the extracellular vesicle described herein can comprise at least one therapeutic compound. In some cases, the at least one therapeutic compound is complexed or anchored by any one of the adapter polypeptide described herein. In some cases, the at least one therapeutic compound is within the extracellular vesicle. Exemplary therapeutic compound includes cancer drug.

Treatment with Extracellular Vesicles

Described herein are methods of treating a disease in a subject by administrating a therapeutically effective amount of the composition or pharmaceutical composition comprising the extracellular vesicles described herein. In some cases, the extracellular vesicle comprises the at least one adapter polypeptide and at least one therapeutic described herein. In some cases, the adapter polypeptide comprises the Fc receptor to be complexed with a Fc region of any one of the antibodies described herein. In some cases, the adapter polypeptide further comprises a targeting domain comprising a tumor homing peptide, a tissue homing peptide, a tissue-targeting domain, a cell-penetrating peptide, a viral membrane protein, and any combination or fragment thereof. In some instances, the antibody and the targeting domain respectively bind to a first and second cell-surface marker associated with a diseased cell, wherein upon binding to the diseased cell the extracellular vesicle delivers the at least one therapeutic to the diseased cell. In some cases, the diseased cell is a cancer cell. In some cases, the diseased cell is a non-cancerous lesion cell. In some instances, the diseased cell is a tumor cell. In some instances, the at least one therapeutic comprises a therapeutic polynucleotide, a therapeutic polypeptide, a therapeutic compound, a cancer drug, or a combination thereof.

In some cases, targeted cell uptake of the therapeutic delivered by the extracellular vesicle comprising the antibody complexed with the at least one adapter polypeptide and the targeting domain is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or higher compared to targeted cell uptake of the therapeutic delivered by the an extracellular vesicle without the antibody complexed with the adapter polypeptide. In some cases, targeted cell uptake of the therapeutic delivered by the extracellular vesicle comprising the antibody complexed with the at least one adapter polypeptide and the targeting domain is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or higher compared to targeted cell uptake of the therapeutic delivered by the an extracellular vesicle without the adapter polypeptide. In some instances, the targeted cell with the increased uptake of the therapeutic delivered by the extracellular vesicle comprising the antibody complexed with the at least one adapter polypeptide and the targeting domain is a cancerous cell, a non-cancerous lesion cell, a cell as part of a tumor, or a cell as part of a tissue.

In some cases, described herein are methods of treating a disease with an extracellular vesicle comprising an adapter polypeptide and a therapeutic polynucleotide as described herein. In some cases, described herein are methods of treating a tumor with an extracellular vesicle comprising an adapter polypeptide and a therapeutic polynucleotide. In some cases, the methods of treating the tumor comprise delivering a therapeutic polynucleotide, a therapeutic polypeptide, a therapeutic compound, a cancer drug, or a combination thereof, via the extracellular vesicles to the tumor cells. Non-limiting examples of the tumor cells that can be treated with the methods described herein include cells of lung cancer, breast cancer, colorectal cancer, prostate cancer, skin cancer, stomach cancer, liver cancer, breast cancer, or brain cancer. In some cases, the cancer cell targeted by the extracellular vesicles represents a subpopulation within a cancer cell population, such as a cancer stem cell.

In some cases, described herein methods of treating a disease by administering the extracellular vesicles comprising adapter polypeptide and therapeutic polynucleotides to a subject in need thereof. In some cases, the extracellular vesicles comprising adapter polypeptide and therapeutic polynucleotides are administered daily, every day, every alternate day, five days a week, once a week, every other week, two weeks per month, three weeks per month, once a month, twice a month, three times per month, or more. The extracellular vesicles comprising adapter polypeptide and therapeutic polynucleotides can be administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the subject's status improves, the dose of the extracellular vesicles comprising adapter polypeptide and therapeutic polynucleotides being administered can be temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday can be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some cases, an effective amount of the extracellular vesicles comprising adapter polypeptide and therapeutic polynucleotides can be administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, or once for longer period of time.

Once improvement of the subject's disease or conditions associated with the disease have occurred, a maintenance dose of extracellular vesicles is administered if necessary. Subsequently, the dosage or the frequency of administration, or both, can be reduced, as a function of the symptoms, to a level at which the improved disease, disorder or condition is retained.

In some cases, the amount of the extracellular vesicles comprising adapter polypeptide and therapeutic polynucleotides that correspond to such an amount varies depending upon factors such as the severity of the disease, the identity (e.g., weight) of the subject or host in need of treatment, but nevertheless is routinely determined in a manner known in the art according to the particular circumstances surrounding the case, including, e.g., the specific extracellular vesicles being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day. In some cases, the dosage can be at least partially determined by occurrence or severity of grade 3 or grade 4 adverse events in the subject.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some cases, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Production of Extracellular Vesicles

Described herein, in some cases, are methods and systems for producing the extracellular vesicle comprising the adapter polypeptide described herein. Also described herein, in some cases, are methods and systems for producing the extracellular vesicle comprising the adapter polypeptide and the at least one therapeutic described herein.

In some cases, the method comprises introducing at least one heterologous polynucleotide into a cell. In some cases, the at least one heterologous polynucleotide is a vector. In some cases, the vector is a plasmid. In some instances, the at least one heterologous polynucleotide introduced into the cell encodes at least one adapter polypeptide described herein. In some cases, the at least one heterologous polynucleotide encodes at least one targeting domain. In some instances, the at least one heterologous polynucleotide comprises at least therapeutic polynucleotide described herein. In some instances, the at least one heterologous polynucleotide encodes the at least one therapeutic polynucleotide described herein. In some instances, the at least one heterologous polynucleotide encodes at least one therapeutic polypeptide described herein.

In some instances, at least two heterologous polynucleotides are introduced into the same cell, where a first heterologous polynucleotide comprising a first vector (e.g., plasmid) encoding at least one adapter polypeptide. In some cases, a second heterologous polynucleotide introduced into the same cell comprises a second vector (e.g., plasmid) encoding the at least one therapeutic polynucleotide or the at least one therapeutic polypeptide. The first and the second heterologous polynucleotide can be introduced into the same cell simultaneously or sequentially. In some cases, the first and the second heterologous polynucleotide can be introduced into the same cell by the same method of transfection. In some cases, the first and the second heterologous polynucleotide can be introduced into the same cell by the different methods of transfection.

In some cases, the heterologous polynucleotide can be introduced into the cell via the use of expression vectors. In the context of an expression vector, the vector can be readily introduced into the cell described herein by any method in the art. For example, the expression vector can be transferred into the cell by biological, chemical, or physical methods of transfection.

Biological methods of transfection for introducing the heterologous polynucleotide of interest into the cell can include the use of DNA and RNA vectors. Viral vectors, and especially retroviral vectors, have become the most widely used method for inserting genes into non-human mammalian cells. Other viral vectors, in some cases, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical methods of transfection for introducing the heterologous polynucleotide into the cell can include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo, or in vivo). In another aspect, the nucleic acid is associated with a lipid. The nucleic acid associated with a lipid, in some cases, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some cases, they are present in a bilayer structure, as micelles, or with a “collapsed” structure. Alternately, they are simply be interspersed in a solution, possibly forming aggregates that are not uniform in size or shape. Lipids are fatty substances which are, in some cases, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some cases, dimyristyl phosphatidylcholine (“DMPC”) is obtained from Sigma, St. Louis, Mo.; in some cases, dicetyl phosphate (“DCP”) is obtained from K & K Laboratories (Plainview, N.Y.); cholesterol (“Choi”), in some cases, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids, in some cases, assume a micellar structure or merely exist as nonuniform aggregates of lipid molecules. Also contemplated are lipofectamine-nucleic acid complexes.

Physical methods of transfection for introducing the heterologous polynucleotide into the cell can include calcium phosphate precipitation, lipofection, particle bombardment, microinjection, gene gun, electroporation, micro-needle array, nano-needle array, sonication, or chemical permeation. Electroporation includes microfluidics electroporation, microchannel electroporation, or nanochannel electroporation. In certain cases, the extracellular vesicle cell is transfected with the at least one heterologous polynucleotide by microchannel electroporation or nanochannel electroporation. In some instances, the microchannel electroporation or the nanochannel electroporation comprises use of micropore patterned silicon wafers, nanopore patterned silicon wafers, track etch membranes, ceramic micropore membranes, ceramic nanopore membranes, other porous materials, or a combination thereof. In some instances, the at least one heterologous polynucleotide or the at least one vector (e.g., plasmid) is nanoelectroporated into the extracellular vesicle donor cell via a nanochannel located on a biochip.

In some cases, extracellular vesicle donor cells can be grown and attached on a surface of a substrate. In some cases, the substrate comprises a biochip. In some cases, the surface of the substrate comprises metallic material. In some cases, the substrates comprise metallic material. Non-limiting examples of metallic material include aluminum (Al), indium tin oxide (ITO, In₂O₃:SnO2), chromium (Cr), gallium arsenide (GaAs), gold (Au), molybdenum (Mo), organic residues and photoresist, platinum (Pt), silicon (Si), silicon dioxide (SiO₂), silicon on insulator (SOI), silicon nitride (Si₃N₄) tantalum (Ta), titanium (Ti), titanium nitride (TiN), tungsten (W). In some cases, the metallic material can be treated or etched to create an array or channels. In some cases, the metallic surface can be treated or etched with phosphoric acid (H₃PO₄), acetic acid, nitric acid (HNO₃), water (H₂O), hydrochloric acid (HCl), (HNO₃), ceric ammonium nitrate ((NH₄)₂Ce(NO₃)₆, citric acid (C₆H₈O₇), hydrogen peroxide (H₂O₂), aqua regia, iodine solution, sulfuric acid (H₂SO₄), hydrofluoric acid (HF), potassium hydroxide (KOH), ethylenediamine pyrocatechol (EDP), tetramethylammonium hydroxide (TMAH), buffered oxide, ammonium fluoride (NH₄F), SCl, Cl₂, CCl₄, SiCl₄, BCl₃, SiCl₄, BCl₃, CCl₂F₂, CF₄, O₂, CF₄, SF₆, NF₃, CHF₃, or a combination thereof.

In some cases, the metallic surface can be treated with a gas or plasma to increase hydrophilicity. In some cases, the metallic surface can be treated with a gas or plasma to increase hydrophobicity. Exemplary gas or plasma for increasing hydrophilicity or hydrophobicity of the metallic surface include oxygen, nitrogen, ammonia, argon, chlorine, fluorine, bromine, iodine, astatine, hydrogen, or a combination thereof.

In some cases, the extracellular vesicle donor cells can be grown and attached to a surface of a substrate made of polymers such as polypropylene, polyethylene, polystyrene, ABS, polyamide, polyethylene copolymer, epoxy, polyester, polyvinylchloride, phenolic, polytetrafluoroethylene, polyethylene copolymer, fluorinated ethylene propylene, polyvinylidene, silicone, natural rubber, latex, polyurethane, styrene butadiene rubber, fluorocarbon copolymer elastomer, polyethylene terephthalate, polycarbonate, polyamide, polyaramid, polyaryl ether ketone, polyacetal, polyphenylene oxide, PBT, polysulfone, polyethersulfone, polyarylsulfone, polyphenylene sulfide, polytetrafluoroethylene, beryllium oxide etc. In some cases, the surface made of polymers can be semi-permeable with at least one pore. In some instances, pore size of the semi-permeable polymer surface can be between about 0.01 μm to about 10 μm. In some embodiment, pore size of the semi-permeable polymer surface can be between about 0.01 μm to about 0.05 μm, about 0.01 μm to about 0.1 μm, about 0.01 μm to about 0.5 μm, about 0.01 μm to about 1 μm, about 0.01 μm to about 5 μm, about 0.01 μm to about 10 μm, about 0.05 μm to about 0.1 μm, about 0.05 μm to about 0.5 μm, about 0.05 μm to about 1 μm, about 0.05 μm to about 5 μm, about 0.05 μm to about 10 μm, about 0.1 μm to about 0.5 μm, about 0.1 μm to about 1 μm, about 0.1 μm to about 5 μm, about 0.1 μm to about 10 μm, about 0.5 μm to about 1 μm, about 0.5 μm to about 5 μm, about 0.5 μm to about 10 μm, about 1 μm to about 5 μm, about 1 μm to about 10 μm, or about 5 μm to about 10 μm. In some embodiment, pore size of the semi-permeable polymer surface can be between about 0.01 μm, about 0.05 μm, about 0.1 μm, about 0.5 μm, about 1 μm, about 5 μm, or about 10 μm. In some embodiment, pore size of the semi-permeable polymer surface can be between at least about 0.01 μm, about 0.05 μm, about 0.1 μm, about 0.5 μm, about 1 μm, or about 5 μm. In some embodiment, pore size of the semi-permeable polymer surface can be between at most about 0.05 μm, about 0.1 μm, about 0.5 μm, about 1 μm, about 5 μm, or about 10 μm.

In some cases, the surface of the polymer can be treated with a gas or plasma to increase hydrophilicity. In some cases, the surface of the polymer can be treated with a gas or plasma to increase hydrophobicity. Exemplary gas or plasma for increasing hydrophilicity or hydrophobicity of the metallic surface include oxygen, nitrogen, ammonia, argon, chlorine, fluorine, bromine, iodine, astatine, hydrogen, or a combination thereof.

### Nanoelectroporation

In some cases, any cell can be electroporated by microchannel electroporation or nanochannel electroporation described herein to become extracellular vesicle donor cell to produce extracellular vesicles described herein. The extracellular vesicle donor cell can be any cell that can be genetically modified or manipulated to produce and secrete extracellular vesicle at a level that is higher than a basal level secretion of the extracellular vesicle. As such, a cell with low or negligible basal level of secretion of extracellular vesicle can also be transfected by microchannel electroporation or nanochannel electroporation to produce and secrete the extracellular vesicle described herein. In some cases, the extracellular vesicle donor cell can be an autologous cell. In such case, the extracellular vesicle donor cell is obtained from a subject who is also receiving, e.g., administered, the extracellular vesicle described herein. In some cases the donor cell is genetically modified (for example, genetically modified with targeting polypeptides on the EV surface and/or therapeutic RNAs in the EVs).

In some cases, extracellular vesicles produced and secreted from an extracellular donor call can induce pro-inflammatory alloimmune responses by T cells, thus posing a challenge for using the extracellular vesicles as therapeutics. In some cases, the extracellular vesicle donor cell can be an allogenic cell, where the extracellular vesicle donor cell is a cell obtained from a source which is of same species but genetically distinct from the subject who is receiving the extracellular vesicle described herein. In some cases, the extracellular vesicle donor cell can be a cell style that produces and secrete allogenic extracellular vesicles. For example, mesenchymal stem cells (MSCs) exhibits hypoimmunogenicity, because of lacking histocompatibility complex class II (MHC-II) and costimulatory molecule expression, allowing MSCs to serve as extracellular vesicle donor cells for producing and secreting allogenic extracellular vesicles that share similar anti-inflammatory and trophic properties as the parental MSCs that produce and secrete the allogenic extracellular vesicles.

In some cases, the extracellular vesicle donor cell to be electroporated by microchannel electroporation or nanochannel electroporation described herein can be any eukaryotic cell. In some instances, the extracellular vesicle donor cell can be cell from a cell line, a stem cell, a primary cell, or a differentiated cell. In some cases, the extracellular vesicle donor cell can be selected from the group consisting of mouse embryonic fibroblast (MEF), human embryonic fibroblast (HEF), dendritic cells mesenchymal stem cell, bone marrow-derived dendritic cell, bone marrow derived stromal cell, adipose stromal cell, endothelial cell, enucleated cell, neural stem cell, immature dendritic cell, and immune cell.

In some cases, the extracellular vesicle donor cell can be a genetically modified cell of any of cell described herein, where at least one heterologous polynucleotide is introduced into the cell. In some cases, the at least one heterologous polynucleotide is transfected into the extracellular vesicle by electroporation. In some cases, the electroporation comprises microchannel electroporation or nanochannel electroporation. In some instances, the at least one heterologous polynucleotide is transfected into the extracellular vesicle by nanochannel electroporation. In some instances, the heterologous polynucleotide transfected into the extracellular vesicle donor cell is integrated into the chromosome of the nanoelectroporated cell. In some cases, the heterologous polynucleotide transfected into the extracellular vesicle donor cell is not integrated into the chromosome of the nanoelectroporated cell. In some cases, the nanoelectroporated extracellular vesicle donor cell is stability transfected with heterologous polynucleotide. In some cases, the nanoelectroporated extracellular vesicle donor cell is transiently transfected with heterologous polynucleotide. In some cases, the transfected extracellular vesicle donor cell can be a cell derived from a cell line. In some instances, the at least one heterologous polynucleotide is a vector. In some cases, the vector is a plasmid.

In some cases, the extracellular vesicle donor cell continuously produces and secretes the extracellular vesicle at a steady or a basal rate. In some cases, the extracellular vesicle donor cell produces and secretes the extracellular vesicle at a basal rate, where additional extracellular vesicle can be produced and secreted by stimulating the cell. For example, the extracellular vesicle donor cell can be stimulated to produce and secret extracellular vesicle at a rate that is higher than the basal rate by heat shocking the extracellular vesicle donor cell or contacting the extracellular vesicle donor cell with Ca²⁺. In some cases, the extracellular vesicle donor cell can be stimulated to produce and secret extracellular vesicle at a rate that is higher than the basal rate by electroporating the at least one heterologous polynucleotide into the cell. In some cases, the extracellular vesicle donor cell can be stimulated to produce and secret extracellular vesicle at a rate that is higher than the basal rate by microchannel electroporation or nanochannel electroporation the at least one heterologous polynucleotide into the cell. In some cases, the extracellular vesicle donor cell can be stimulated to produce and secrete extracellular vesicle at a rate that is higher than the basal rate by nanochannel electroporating the at least one heterologous polynucleotide into the cell. In some instances, the extracellular vesicle donor cell stimulated by nanochannel electroporation can produce and secrete the extracellular vesicle at a rate that is at least 0.1 fold, 0.2 fold, 0.3 fold, 0.4 fold, 0.5 fold, 0.6 fold, 0.7 fold, 0.8 fold, 0.9 fold, 2 folds, 5 folds, 10 folds, 50 folds, 100 folds, 500 folds, 1,000 folds, 5,000 folds, 10,000 fold, 50,000 folds, 100.000 fold, or more higher than the basal rate of the extracellular vesicle donor cell producing and secreting the extracellular vesicle. In some cases, the extracellular vesicle donor cell stimulated by nanochannel electroporation can produce and secrete the extracellular vesicle at a rate that is at least 0.1 fold, 0.2 fold, 0.3 fold, 0.4 fold, 0.5 fold, 0.6 fold, 0.7 fold, 0.8 fold, 0.9 fold, 2 folds, 5 folds, 10 folds, 50 folds, 100 folds, 500 folds, 1,000 folds, 5,000 folds, 10,000 fold, 50,000 folds, 100.000 fold, or more higher than the rate of the extracellular vesicle donor cell stimulated by methods of transfection other than nanoelectroporation for producing and secreting the extracellular vesicle.

The extracellular vesicles produced and secreted by the extracellular vesicle donor cells can be collected and purified by centrifugation or ultracentrifugation, where extracellular vesicles are purified from other cellular debris or molecules. In some cases, the extracellular vesicles produced and secreted by the extracellular vesicle donor cells can be collected and purified by tangential flow filtration, where other cellular debris or molecules other than the extracellular vesicles described herein can be continuously removed.

In some cases, the heterologous polynucleotide transfected into the extracellular vesicle donor cell encodes at least one adapter polypeptide described herein. In some cases, the heterologous polynucleotide transfected into the extracellular vesicle donor cell encodes at least one therapeutic described herein. In some cases, the therapeutic is a therapeutic polynucleotide. In some instances, the therapeutic is a therapeutic polypeptide. In some instances, the extracellular vesicle donor cell transfected with the at least one heterologous polynucleotide produces and secretes extracellular vesicle comprising the at least one adapter polypeptide. In some instances, the extracellular vesicle donor cell transfected with at least one heterologous polynucleotide produces and secretes extracellular vesicle comprising the at least one therapeutic. In some instances, the extracellular vesicle donor cell transfected with at least one heterologous polynucleotide produces and secretes extracellular vesicle comprising the at least one adapter polypeptide and the at least one therapeutic.

In some cases, the heterologous polynucleotide transfected into the extracellular vesicle donor cell is a vector (e.g., plasmid). In some cases, the heterologous polynucleotide encodes at least one adapter polypeptide described herein. In some cases, the at least one adapter polypeptide comprises a peptide sequence of the Fc binding domain, Fc receptor, or a fragment thereof described herein. In some instances, the at least one adapter polypeptide comprises a peptide sequence of an extracellular domain. In some cases, the at least one adapter polypeptide comprises a peptide sequence of a targeting domain that is attached to the extracellular domain of the adapter polypeptide.

In some cases, the nanoelectroporated extracellular vesicle donor cell produces and secretes the extracellular vesicle comprising the at least one therapeutic that is expressed on an extracellular surface of the extracellular vesicle. In some cases, the nanoelectroporated extracellular vesicle donor cell produces and secretes the extracellular vesicle comprising the at least one therapeutic that is expressed on the surface of the extracellular vesicle by attaching the at least one therapeutic to the adapter polypeptide. In some cases, the nanoelectroporated extracellular vesicle donor cell produces and secretes the extracellular vesicle comprising the at least one therapeutic that is expressed on the surface of the extracellular vesicle by attaching the at least one therapeutic to the extracellular domain of the adapter polypeptide. In some cases, the nanoelectroporated extracellular vesicle donor cell produces and secretes the extracellular vesicle comprising the at least one therapeutic that is expressed and inserted into the membrane of the extracellular vesicle. In some cases, the nanoelectroporated extracellular vesicle donor cell produces and secretes the extracellular vesicle comprising the at least one therapeutic that is within the extracellular vesicle.

In some cases, the extracellular vesicle produced and secreted by the nanoelectroporated extracellular vesicle donor cell is any membrane-bound particle. In some cases, the extracellular vesicle produced and secreted by the nanoelectroporated extracellular vesicle donor cell is an exosome, a microvesicle, a retrovirus-like particle, an apoptotic body, an apoptosome, an oncosome, an exopher, an enveloped virus, an exomere, or other very large extracellular vesicle. In some cases, the extracellular vesicle produced and secreted by the nanoelectroporated extracellular vesicle donor cell is an exosome.

In some cases, cells grown or attached to the metallic or polymer surface can be nanoelectroporated by nanoelectroporation systems described herein. In some cases, the system comprises a fluidic chamber with an upper boundary and a lower boundary. The placement of the substrate with the cells in the fluid chamber create an upper chamber and a lower chamber. In some cases, the system further comprises at least one nanochannel. In some cases, the nanochannel can be embedded within the substrate. In some cases, the nanochannel comprises pores of the semi-permeable polymer substrate. In some embodiment, the nanochannel comprises a height from about 0.01 μm to about 500 μm. In some embodiment, the nanochannel comprises a height from about 0.01 μm to about 0.05 μm, about 0.01 μm to about 0.1 μm, about 0.01 μm to about 0.5 μm, about 0.01 μm to about 1 μm, about 0.01 μm to about 2 μm, about 0.01 μm to about 5 μm, about 0.01 μm to about 10 μm, about 0.01 μm to about 20 μm, about 0.01 μm to about 50 μm, about 0.01 μm to about 100 μm, about 0.01 μm to about 500 μm, about 0.05 μm to about 0.1 μm, about 0.05 μm to about 0.5 μm, about 0.05 μm to about 1 μm, about 0.05 μm to about 2 μm, about 0.05 μm to about 5 μm, about 0.05 μm to about 10 μm, about 0.05 μm to about 20 μm, about 0.05 μm to about 50 μm, about 0.05 μm to about 100 μm, about 0.05 μm to about 500 μm, about 0.1 μm to about 0.5 μm, about 0.1 μm to about 1 μm, about 0.1 μm to about 2 μm, about 0.1 μm to about 5 μm, about 0.1 μm to about 10 μm, about 0.1 μm to about 20 μm, about 0.1 μm to about 50 μm, about 0.1 μm to about 100 μm, about 0.1 μm to about 500 μm, about 0.5 μm to about 1 μm, about 0.5 μm to about 2 μm, about 0.5 μm to about 5 μm, about 0.5 μm to about 10 μm, about 0.5 μm to about 20 μm, about 0.5 μm to about 50 μm, about 0.5 μm to about 100 μm, about 0.5 μm to about 500 μm, about 1 μm to about 2 μm, about 1 μm to about 5 μm, about 1 μm to about 10 μm, about 1 μm to about 20 μm, about 1 μm to about 50 μm, about 1 μm to about 100 μm, about 1 μm to about 500 μm, about 2 μm to about 5 μm, about 2 μm to about 10 μm, about 2 μm to about 20 μm, about 2 μm to about 50 μm, about 2 μm to about 100 μm, about 2 μm to about 500 μm, about 5 μm to about 10 μm, about 5 μm to about 20 μm, about 5 μm to about 50 μm, about 5 μm to about 100 μm, about 5 μm to about 500 μm, about 10 μm to about 20 μm, about 10 μm to about 50 μm, about 10 μm to about 100 μm, about 10 μm to about 500 μm, about 20 μm to about 50 μm, about 20 μm to about 100 μm, about 20 μm to about 500 μm, about 50 μm to about 100 μm, about 50 μm to about 500 μm, or about 100 μm to about 500 μm. In some embodiment, the nanochannels comprise a height from about 0.01 μm, about 0.05 μm, about 0.1 μm, about 0.5 μm, about 1 μm, about 2 μm, about 5 μm, about 10 μm, about 20 μm, about 50 μm, about 100 μm, or about 500 μm. In some embodiment, the nanochannels comprise a height from at least about 0.01 μm, about 0.05 μm, about 0.1 μm, about 0.5 μm, about 1 μm, about 2 μm, about 5 μm, about 10 μm, about 20 μm, about 50 μm, or about 100 μm. In some embodiment, the nanochannels comprise a height from at most about 0.05 μm, about 0.1 μm, about 0.5 μm, about 1 μm, about 2 μm, about 5 μm, about 10 μm, about 20 μm, about 50 μm, about 100 μm, or about 500 μm. In some cases, the heights of the nanochannel can be the same. In some cases, the heights of the nanochannel can be the different. In some cases, the heights of the nanochannel should be great enough to accelerate the molecules being nanoelectroporated in the high electric field zone (e.g., inside the nanochannel), but also small enough to enable large molecules being nanoelectroporated to squeeze through in a brief electric pulse.

In some embodiment, the nanochannel comprises a diameter from about 0.01 nm to about 10,000 nm. In some embodiment, the nanochannels comprise a diameter from about 0.01 nm to about 0.1 nm, about 0.01 nm to about 0.5 nm, about 0.01 nm to about 1 nm, about 0.01 nm to about 5 nm, about 0.01 nm to about 10 nm, about 0.01 nm to about 50 nm, about 0.01 nm to about 100 nm, about 0.01 nm to about 500 nm, about 0.01 nm to about 1,000 nm, about 0.01 nm to about 5,000 nm, about 0.01 nm to about 10,000 nm, about 0.1 nm to about 0.5 nm, about 0.1 nm to about 1 nm, about 0.1 nm to about 5 nm, about 0.1 nm to about 10 nm, about 0.1 nm to about 50 nm, about 0.1 nm to about 100 nm, about 0.1 nm to about 500 nm, about 0.1 nm to about 1,000 nm, about 0.1 nm to about 5,000 nm, about 0.1 nm to about 10,000 nm, about 0.5 nm to about 1 nm, about 0.5 nm to about 5 nm, about 0.5 nm to about 10 nm, about 0.5 nm to about 50 nm, about 0.5 nm to about 100 nm, about 0.5 nm to about 500 nm, about 0.5 nm to about 1,000 nm, about 0.5 nm to about 5,000 nm, about 0.5 nm to about 10,000 nm, about 1 nm to about 5 nm, about 1 nm to about 10 nm, about 1 nm to about 50 nm, about 1 nm to about 100 nm, about 1 nm to about 500 nm, about 1 nm to about 1,000 nm, about 1 nm to about 5,000 nm, about 1 nm to about 10,000 nm, about 5 nm to about 10 nm, about 5 nm to about 50 nm, about 5 nm to about 100 nm, about 5 nm to about 500 nm, about 5 nm to about 1,000 nm, about 5 nm to about 5,000 nm, about 5 nm to about 10,000 nm, about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 50 nm to about 100 nm, about 50 nm to about 500 nm, about 50 nm to about 1,000 nm, about 50 nm to about 5,000 nm, about 50 nm to about 10,000 nm, about 100 nm to about 500 nm, about 100 nm to about 1,000 nm, about 100 nm to about 5,000 nm, about 100 nm to about 10,000 nm, about 500 nm to about 1,000 nm, about 500 nm to about 5,000 nm, about 500 nm to about 10,000 nm, about 1,000 nm to about 5,000 nm, about 1,000 nm to about 10,000 nm, or about 5,000 nm to about 10,000 nm. In some embodiment, the nanochannels comprise a diameter from about 0.01 nm, about 0.1 nm, about 0.5 nm, about 1 nm, about 5 nm, about 10 nm, about 50 nm, about 100 nm, about 500 nm, about 1,000 nm, about 5,000 nm, or about 10,000 nm. In some embodiment, the nanochannel comprises a diameter from at least about 0.01 nm, about 0.1 nm, about 0.5 nm, about 1 nm, about 5 nm, about 10 nm, about 50 nm, about 100 nm, about 500 nm, about 1,000 nm, or about 5,000 nm. In some embodiment, the nanochannel comprises a diameter from at most about 0.1 nm, about 0.5 nm, about 1 nm, about 5 nm, about 10 nm, about 50 nm, about 100 nm, about 500 nm, about 1,000 nm, about 5,000 nm, or about 10,000 nm. In some cases, the diameters of the nanochannel can be the same. In some cases, the diameters of the nanochannel can be the different.

In some cases, the nanochannels can be arranged into a nanochannel array. In some cases, the nanochannels can be arranged into a nanochannel array with spacing between the nanochannels. In some instances, the spacing between the nanochannels can be from about 0.01 μm to about 5,000 μm. In some instances, the spacing between the nanochannels can be from about 0.01 μm to about 0.05 μm, about 0.01 μm to about 0.1 μm, about 0.01 μm to about 0.5 μm, about 0.01 μm to about 1 μm, about 0.01 μm to about 5 μm, about 0.01 μm to about 10 μm, about 0.01 μm to about 50 μm, about 0.01 μm to about 100 μm, about 0.01 μm to about 500 μm, about 0.01 μm to about 1,000 μm, about 0.01 μm to about 5,000 μm, about 0.05 μm to about 0.1 μm, about 0.05 μm to about 0.5 μm, about 0.05 μm to about 1 μm, about 0.05 μm to about 5 μm, about 0.05 μm to about 10 μm, about 0.05 μm to about 50 μm, about 0.05 μm to about 100 μm, about 0.05 μm to about 500 μm, about 0.05 μm to about 1,000 μm, about 0.05 μm to about 5,000 μm, about 0.1 μm to about 0.5 μm, about 0.1 μm to about 1 μm, about 0.1 μm to about 5 μm, about 0.1 μm to about 10 μm, about 0.1 μm to about 50 μm, about 0.1 μm to about 100 μm, about 0.1 μm to about 500 μm, about 0.1 μm to about 1,000 μm, about 0.1 μm to about 5,000 μm, about 0.5 μm to about 1 μm, about 0.5 μm to about 5 μm, about 0.5 μm to about 10 μm, about 0.5 μm to about 50 μm, about 0.5 μm to about 100 μm, about 0.5 μm to about 500 μm, about 0.5 μm to about 1,000 μm, about 0.5 μm to about 5,000 μm, about 1 μm to about 5 μm, about 1 μm to about 10 μm, about 1 μm to about 50 μm, about 1 μm to about 100 μm, about 1 μm to about 500 μm, about 1 μm to about 1,000 μm, about 1 μm to about 5,000 μm, about 5 μm to about 10 μm, about 5 μm to about 50 μm, about 5 μm to about 100 μm, about 5 μm to about 500 μm, about 5 μm to about 1,000 μm, about 5 μm to about 5,000 μm, about 10 μm to about 50 μm, about 10 μm to about 100 μm, about 10 μm to about 500 μm, about 10 μm to about 1,000 μm, about 10 μm to about 5,000 μm, about 50 μm to about 100 μm, about 50 μm to about 500 μm, about 50 μm to about 1,000 μm, about 50 μm to about 5,000 μm, about 100 μm to about 500 μm, about 100 μm to about 1,000 μm, about 100 μm to about 5,000 μm, about 500 μm to about 1,000 μm, about 500 μm to about 5,000 μm, or about 1,000 μm to about 5,000 μm. In some instances, the spacing between the nanochannels can be from about 0.01 μm, about 0.05 μm, about 0.1 μm, about 0.5 μm, about 1 μm, about 5 μm, about 10 μm, about 50 μm, about 100 μm, about 500 μm, about 1,000 μm, or about 5,000 μm. In some instances, the spacing between the nanochannels can be from at least about 0.01 μm, about 0.05 μm, about 0.1 μm, about 0.5 μm, about 1 μm, about 5 μm, about 10 μm, about 50 μm, about 100 μm, about 500 μm, or about 1,000 μm. In some instances, the spacing between the nanochannels can be from at most about 0.05 μm, about 0.1 μm, about 0.5 μm, about 1 μm, about 5 μm, about 10 μm, about 50 μm, about 100 μm, about 500 μm, about 1,000 μm, or about 5,000 μm.

In some cases, the nanoelectroporating system comprises upper and lower electrode layers for generating an electric field within the fluidic chamber. In some cases, the electric field generated by the electrodes for nanoelectroporation comprises an electric field strength from about 0.1 volt/mm to about 50,000 volt/mm. In some cases, the electric field generated by the electrodes for nanoelectroporation comprises an electric field strength from about 0.1 volt/mm to about 0.5 volt/mm, about 0.1 volt/mm to about 1 volt/mm, about 0.1 volt/mm to about 5 volt/mm, about 0.1 volt/mm to about 10 volt/mm, about 0.1 volt/mm to about 50 volt/mm, about 0.1 volt/mm to about 100 volt/mm, about 0.1 volt/mm to about 500 volt/mm, about 0.1 volt/mm to about 1,000 volt/mm, about 0.1 volt/mm to about 5,000 volt/mm, about 0.1 volt/mm to about 10,000 volt/mm, about 0.1 volt/mm to about 50,000 volt/mm, about 0.5 volt/mm to about 1 volt/mm, about 0.5 volt/mm to about 5 volt/mm, about 0.5 volt/mm to about 10 volt/mm, about 0.5 volt/mm to about 50 volt/mm, about 0.5 volt/mm to about 100 volt/mm, about 0.5 volt/mm to about 500 volt/mm, about 0.5 volt/mm to about 1,000 volt/mm, about 0.5 volt/mm to about 5,000 volt/mm, about 0.5 volt/mm to about 10,000 volt/mm, about 0.5 volt/mm to about 50,000 volt/mm, about 1 volt/mm to about 5 volt/mm, about 1 volt/mm to about 10 volt/mm, about 1 volt/mm to about 50 volt/mm, about 1 volt/mm to about 100 volt/mm, about 1 volt/mm to about 500 volt/mm, about 1 volt/mm to about 1,000 volt/mm, about 1 volt/mm to about 5,000 volt/mm, about 1 volt/mm to about 10,000 volt/mm, about 1 volt/mm to about 50,000 volt/mm, about 5 volt/mm to about 10 volt/mm, about 5 volt/mm to about 50 volt/mm, about 5 volt/mm to about 100 volt/mm, about 5 volt/mm to about 500 volt/mm, about 5 volt/mm to about 1,000 volt/mm, about 5 volt/mm to about 5,000 volt/mm, about 5 volt/mm to about 10,000 volt/mm, about 5 volt/mm to about 50,000 volt/mm, about 10 volt/mm to about 50 volt/mm, about 10 volt/mm to about 100 volt/mm, about 10 volt/mm to about 500 volt/mm, about 10 volt/mm to about 1,000 volt/mm, about 10 volt/mm to about 5,000 volt/mm, about 10 volt/mm to about 10,000 volt/mm, about 10 volt/mm to about 50,000 volt/mm, about 50 volt/mm to about 100 volt/mm, about 50 volt/mm to about 500 volt/mm, about 50 volt/mm to about 1,000 volt/mm, about 50 volt/mm to about 5,000 volt/mm, about 50 volt/mm to about 10,000 volt/mm, about 50 volt/mm to about 50,000 volt/mm, about 100 volt/mm to about 500 volt/mm, about 100 volt/mm to about 1,000 volt/mm, about 100 volt/mm to about 5,000 volt/mm, about 100 volt/mm to about 10,000 volt/mm, about 100 volt/mm to about 50,000 volt/mm, about 500 volt/mm to about 1,000 volt/mm, about 500 volt/mm to about 5,000 volt/mm, about 500 volt/mm to about 10,000 volt/mm, about 500 volt/mm to about 50,000 volt/mm, about 1,000 volt/mm to about 5,000 volt/mm, about 1,000 volt/mm to about 10,000 volt/mm, about 1,000 volt/mm to about 50,000 volt/mm, about 5,000 volt/mm to about 10,000 volt/mm, about 5,000 volt/mm to about 50,000 volt/mm, or about 10,000 volt/mm to about 50,000 volt/mm. In some cases, the electric field generated by the electrodes for nanoelectroporation comprises an electric field strength from about 0.1 volt/mm, about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, about 10,000 volt/mm, or about 50,000 volt/mm. In some cases, the electric field generated by the electrodes for nanoelectroporation comprises an electric field strength from at least about 0.1 volt/mm, about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, or about 10,000 volt/mm. In some cases, the electric field generated by the electrodes for nanoelectroporation comprises an electric field strength from at most about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, about 10,000 volt/mm, or about 50,000 volt/mm.

In some instances, the electric field generated by the electrodes for nanoelectroporation comprises a plurality of pulses with pulse duration from about 0.01 millisecond/pulse to about 5,000 millisecond/pulse. In some instances, the electric field generated by the electrodes for nanoelectroporation comprises a plurality of pulses with pulse duration from about 0.01 millisecond/pulse to about 0.05 millisecond/pulse, about 0.01 millisecond/pulse to about 0.1 millisecond/pulse, about 0.01 millisecond/pulse to about 0.5 millisecond/pulse, about 0.01 millisecond/pulse to about 1 millisecond/pulse, about 0.01 millisecond/pulse to about 5 millisecond/pulse, about 0.01 millisecond/pulse to about 10 millisecond/pulse, about 0.01 millisecond/pulse to about 50 millisecond/pulse, about 0.01 millisecond/pulse to about 100 millisecond/pulse, about 0.01 millisecond/pulse to about 500 millisecond/pulse, about 0.01 millisecond/pulse to about 1,000 millisecond/pulse, about 0.01 millisecond/pulse to about 5,000 millisecond/pulse, about 0.05 millisecond/pulse to about 0.1 millisecond/pulse, about 0.05 millisecond/pulse to about 0.5 millisecond/pulse, about 0.05 millisecond/pulse to about 1 millisecond/pulse, about 0.05 millisecond/pulse to about 5 millisecond/pulse, about 0.05 millisecond/pulse to about 10 millisecond/pulse, about 0.05 millisecond/pulse to about 50 millisecond/pulse, about 0.05 millisecond/pulse to about 100 millisecond/pulse, about 0.05 millisecond/pulse to about 500 millisecond/pulse, about 0.05 millisecond/pulse to about 1,000 millisecond/pulse, about 0.05 millisecond/pulse to about 5,000 millisecond/pulse, about 0.1 millisecond/pulse to about 0.5 millisecond/pulse, about 0.1 millisecond/pulse to about 1 millisecond/pulse, about 0.1 millisecond/pulse to about 5 millisecond/pulse, about 0.1 millisecond/pulse to about 10 millisecond/pulse, about 0.1 millisecond/pulse to about 50 millisecond/pulse, about 0.1 millisecond/pulse to about 100 millisecond/pulse, about 0.1 millisecond/pulse to about 500 millisecond/pulse, about 0.1 millisecond/pulse to about 1,000 millisecond/pulse, about 0.1 millisecond/pulse to about 5,000 millisecond/pulse, about 0.5 millisecond/pulse to about 1 millisecond/pulse, about 0.5 millisecond/pulse to about 5 millisecond/pulse, about 0.5 millisecond/pulse to about 10 millisecond/pulse, about 0.5 millisecond/pulse to about 50 millisecond/pulse, about 0.5 millisecond/pulse to about 100 millisecond/pulse, about 0.5 millisecond/pulse to about 500 millisecond/pulse, about 0.5 millisecond/pulse to about 1,000 millisecond/pulse, about 0.5 millisecond/pulse to about 5,000 millisecond/pulse, about 1 millisecond/pulse to about 5 millisecond/pulse, about 1 millisecond/pulse to about 10 millisecond/pulse, about 1 millisecond/pulse to about 50 millisecond/pulse, about 1 millisecond/pulse to about 100 millisecond/pulse, about 1 millisecond/pulse to about 500 millisecond/pulse, about 1 millisecond/pulse to about 1,000 millisecond/pulse, about 1 millisecond/pulse to about 5,000 millisecond/pulse, about 5 millisecond/pulse to about 10 millisecond/pulse, about 5 millisecond/pulse to about 50 millisecond/pulse, about 5 millisecond/pulse to about 100 millisecond/pulse, about 5 millisecond/pulse to about 500 millisecond/pulse, about 5 millisecond/pulse to about 1,000 millisecond/pulse, about 5 millisecond/pulse to about 5,000 millisecond/pulse, about 10 millisecond/pulse to about 50 millisecond/pulse, about 10 millisecond/pulse to about 100 millisecond/pulse, about 10 millisecond/pulse to about 500 millisecond/pulse, about 10 millisecond/pulse to about 1,000 millisecond/pulse, about 10 millisecond/pulse to about 5,000 millisecond/pulse, about 50 millisecond/pulse to about 100 millisecond/pulse, about 50 millisecond/pulse to about 500 millisecond/pulse, about 50 millisecond/pulse to about 1,000 millisecond/pulse, about 50 millisecond/pulse to about 5,000 millisecond/pulse, about 100 millisecond/pulse to about 500 millisecond/pulse, about 100 millisecond/pulse to about 1,000 millisecond/pulse, about 100 millisecond/pulse to about 5,000 millisecond/pulse, about 500 millisecond/pulse to about 1,000 millisecond/pulse, about 500 millisecond/pulse to about 5,000 millisecond/pulse, or about 1,000 millisecond/pulse to about 5,000 millisecond/pulse. In some instances, the electric field generated by the electrodes for nanoelectroporation comprises a plurality of pulses with pulse duration from about 0.01 millisecond/pulse, about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, about 1,000 millisecond/pulse, or about 5,000 millisecond/pulse. In some instances, the electric field generated by the electrodes for nanoelectroporation comprises a plurality of pulses with pulse duration from at least about 0.01 millisecond/pulse, about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, or about 1,000 millisecond/pulse. In some instances, the electric field generated by the electrodes for nanoelectroporation comprises a plurality of pulses with pulse duration from at most about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, about 1,000 millisecond/pulse, or about 5,000 millisecond/pulse. In some cases, the nanoelectroporation comprises 1 pulse, 2 pulses, 3 pulses, 4 pulses, 5 pulses, 6 pulses, 7 pulses, 8 pulses, 9 pulses, 10 pulses, 11 pulses, 12 pulses, 13 pulses, 14 pulses, 15 pulses, 16 pulses, 17 pulses, 18 pulses, 19 pulses, 20 pulses or more.

In some cases, the methods and systems of producing the extracellular vesicles comprising the adapter polypeptides and the therapeutic polynucleotides comprise loading the nanochannels with the plurality of heterologous polynucleotides (such as vectors) to be nanoelectroporated into the cells. In some cases, molecules other than polynucleotides (e.g. proteins, biomolecules, compounds, etc) can be loaded into the nanochannels to be nanoelectroporated into the cells. In some cases, the electric field generated by the upper and the lower electrodes accelerate the vectors (e.g., plasmids) into the cells. In some cases, the electric field generated for nanoelectroporation creates pores in the cells of the membrane to allow the nanoelectroporation of the vectors (e.g., plasmids). In some cases, the pores in the membrane of the extracellular vesicle donor cells can be formed at a focal point, e.g. exit of the nanochannel where the electric field directly contacts the cell membrane.

In some cases, an nanoelectroporated extracellular vesicle donor cell can produce and secrete at least 10%, 50%, 1 fold, 5 fold, 10 fold, 50 fold, 100 fold, 500 fold, 1000 fold, 5000 fold, or more extracellular vesicles than an extracellular vesicle donor cell transfected by non-nanoelectroporation (e.g. conventional bulk electroporation, gene gun, lipofectamine transfection, etc.)

In some instances, extracellular vesicles produced and secreted by nanoelectroporated extracellular vesicle donor cell comprises at least 50%, 1 fold, 2 fold, 5 fold, 100 fold, 500 fold, 1000 fold, or more therapeutic polynucleotide compared to extracellular vesicles produced and secreted by an extracellular vesicle donor cell transfected by non-nanoelectroporation. In some cases, the therapeutic polynucleotides encapsulated by the extracellular vesicle produced and secreted by the nanoelectroporated extracellular vesicle donor cell are at least 10%, 20%, 30%, 40%, 50%, 60%, 70%, 80%, 90%, 95%, 99%, or more likely to be intact for encoding therapeutic polypeptide than therapeutic polynucleotide encapsulated by the extracellular vesicles produced and secreted by an extracellular vesicle donor cell transfected by non-nanoelectroporation.

### Vaccines

Described herein are compositions comprising an extracellular vesicle described herein. In some cases, the extracellular vesicle comprises at least one adapter polypeptide comprising a peptide sequence that is at 40%, 50%, 60%, 70%, 80%, 90%, 95%, or 99% identical to a peptide sequence of any one of the Fc receptors described herein. In some instances, the at least one adapter polypeptide comprises a peptide sequence that is at least 40%, 50%, 60%, 70%, 80%, 90%, 95%, or 99% identical to a peptide sequence of any one of the Fc receptors: FcγRI (CD64), FcγRII (CD32), or FcγRIII (CD16). In some instances, the at least one adapter polypeptide comprises a peptide sequence that is at least 40%, 50%, 60%, 70%, 80%, 90%, 95%, or 99% identical to a peptide sequence of Fc receptor FcγRI (CD64). In some cases, the extracellular vesicle comprises an antibody complexed with the adapter polypeptide. In some cases, the antibody binds to a first cell-surface marker of an immune cell. In some instances, the extracellular vesicle further comprises at least one viral mimic peptide. The at least one viral mimic peptide can trigger an immune response that results in adaptive immunity against the virus where the viral mimic peptide is derived from. In some cases, the viral mimic peptide is attached to an extracellular domain of the adapter polypeptide.

In some cases, the adapter polypeptide comprises a targeting domain attached to the extracellular domain of the adapter polypeptide. In some cases, targeting domain binds to a second cell-surface marker associated with the same immune cell. In some instance, the immune cell is a myeloid cell, a T cell such as alpha beta cytotoxic T cell, a gamma delta T cell, a regulatory T cell, a natural killer T cell, a B cell, a helper T cell, macrophages, mast cells, a phagocyte, a lymphoid cell, a granulocyte, a macrophage, or a dendritic cell. In some cases, the immune cell is a T cell, a B cell, a dendritic cell, a macrophage, or a natural killer (NK) cell.

In some instances, the first cell-surface marker comprises C5aR, CD10, CD107, CD11, CD117, CD123, CD125, CD135, CD138/Syndecan-1, CD14, CD16, CD163, CD18, CD19, CD193, CD20, CD203, CD206, CD21, CD22, CD23, CD235, CD25, CD3, CD32, CD33, CD34, CD36, CD38, CD4, CD41, CD42, CD44, CD45, CD45R, CD45RA, CD49, CD55, CD56, CD61, CD65, CD68, CD7, CD71, CD8, CD9, CD90/Thyl, CD94, Clusterin, CXCR3B-specific, F4/80, FcεRI, Glycophorin A, GP9, GZMB, HBEI-Specific, HLA-DR, IL3Ra, Integrin alpha-4, Integrin beta-1, Integrin beta-3, LILRA4, NKp4, P-selectin, Siglec-8, or VEGFR-1/FLT-1. In some cases, the first cell-surface marker comprises LILRA4, CD3, CD19, CD20, or CD28.

In some cases, the antibody complexed with the at least one adapter polypeptide is a monoclonal antibody. In some instances, the antibody is a humanized antibody. In some cases, the antibody is a humanized monoclonal antibody. In some cases, the antibody is an IgG. In some cases, the antibody is IgG1 or IgG3. In some cases, the antibody comprises a Fc region to be complexed with the adapter polypeptide comprising the Fc receptor. In some cases, the antibody is non-covalently complexed with the adapter polypeptide.

In some cases, the at least one viral mimic peptide is expressed on the extracellular surface of the extracellular vesicle. In some cases, the at least one viral mimic peptide is partially inserted into the membrane of the extracellular vesicle. In some cases, the at least one viral mimic peptide is attached to the extracellular domain of the adapter polypeptide. In some cases, both the at least one viral mimic peptide and the targeting domain are attached to the same extracellular domain of the adapter polypeptide. In some cases, the at least one viral mimic peptide is attached to the extracellular domain of a first adapter polypeptide, while the targeting domain is attached to a separate extracellular domain of a second adapter polypeptide.

In some cases, the viral mimic peptide is derived from a viral protein of a virus. The virus can be a DNA virus or an RNA virus. A DNA virus can be a single-stranded (ss) DNA virus, a double-stranded (ds) DNA virus, or a DNA virus that contains both ss and ds DNA regions. An RNA virus can be a single-stranded (ss) RNA virus or a double-stranded (ds) RNA virus. A ssRNA virus can further be classified into a positive-sense RNA virus or a negative-sense RNA virus.

In some cases, the viral mimic peptide is derived from a coronavirus protein of the Coronaviridae family. The Coronaviridae family can include alphacoronavirus, betacoronavirus, deltacoronavirus, or gammacoronavirus. In some cases, the coronavirus includes MERS-COV, SARS-COV, or SARS-COV-2. In some cases, the coronavirus protein is a SARS-COV-2 viral protein. In some instances, the viral mimic peptide is derived from a viral protein encoded by a nucleic acid sequence provided in SEQ ID NO: 1. In some instances, the viral mimic peptide is derived from the SARS-COV-2 viral protein is selected from the group consisting of: orfla, orflab, Spike protein (S protein), 3a, 3b, Envelope protein (E protein), Membrane protein (M protein), p6, 7a, 7b, 8b, 9b, Nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase).

In some instances, the viral mimic peptide is derived from a viral protein that at least about 70%, 75%, 80%, 85%, 90%, 95%, or 99% identical to any one of SEQ ID NOS: 2-5. In some instances, the viral mimic peptide comprises a peptide sequence of SEQ ID NOS: 6-10.

| | | Type of |
|---|---|---|
| # | Sequence | Sequence |
| 1 | TTGATTGGTGATTGTGCAAC | >gi\|17981 |
| | TGTACATACAGCTAATAAAT | 74254\|ref\| |
| | GGGATCTCATTATTAGTGAT | NC_0455 |
| | ATGTACGACCCTAAGACTAA | 12.2\|: |
| | AAATGTTACAAAAGAAAATG | 20989-25956 |
| | ACTCTAAAGAGGGTTTTTTC | Severe |
| | ACTTACATTTGTGGGTTTAT | acute |
| | ACAACAAAAGCTAGCTCTTG | respiratory |
| | GAGGTTCCGTGGCTATAAAG | syndrome |
| | ATAACAGAACATTCTTGGAA | coronavirus |
| | TGCTGATCTTTATAAGCTCA | 2 |
| | TGGGACACTTCGCATGGTGG | isolate |
| | ACAGCCTTTGTTACTAATGT | Wuhan- |
| | GAATGCGTCATCATCTGAAG | Hu-1, |
| | CATTTTTAATTGGATGTAAT | complete |
| | TATCTTGGCAAACCACGCGA | genome |
| | ACAAATAGATGGTTATGTCA | |
| | TGCATGCAAATTACATATTT | |
| | TGGAGGAATACAAATCCAAT | |
| | TCAGTTGTCTTCCTATTCTT | |
| | TATTTGACATGAGTAAATTT | |
| | CCCCTTAAATTAAGGGGTAC | |
| | TGCTGTTATGTCTTTAAAAG | |
| | AAGGTCAAATCAATGATATG | |
| | ATTTTATCTCTTCTTAGTAA | |
| | AGGTAGACTTATAATTAGAG | |
| | AAAACAACAGAGTTGTTATT | |
| | TCTAGTGATGTTCTTGTTAA | |
| | CAACTAAACGAACAATGTTT | |
| | GTTTTTCTTGTTTTATTGCC | |
| | ACTAGTCTCTAGTCAGTGTG | |
| | TTAATCTTACAACCAGAACT | |
| | CAATTACCCCCTGCATACAC | |
| | TAATTCTTTCACACGTGGTG | |
| | TTTATTACCCTGACAAAGTT | |
| | TTCAGATCCTCAGTTTTACA | |
| | TTCAACTCAGGACTTGTTCT | |
| | TACCTTTCTTTTCCAATGTT | |
| | ACTTGGTTCCATGCTATACA | |
| | TGTCTCTGGGACCAATGGTA | |
| | CTAAGAGGTTTGATAACCCT | |
| | GTCCTACCATTTAATGATGG | |
| | TGTTTATTTTGCTTCCACTG | |
| | AGAAGTCTAACATAATAAGA | |
| | GGCTGGATTTTTGGTACTAC | |
| | TTTAGATTCGAAGACCCAGT | |
| | CCCTACTTATTGTTAATAAC | |
| | GCTACTAATGTTGTTATTAA | |
| | AGTCTGTGAATTTCAATTTT | |
| | GTAATGATCCATTTTTGGGT | |
| | GTTTATTACCACAAAAACAA | |
| | CAAAAGTTGGATGGAAAGTG | |
| | AGTTCAGAGTTTATTCTAGT | |
| | GCGAATAATTGCACTTTTGA | |
| | ATATGTCTCTCAGCCTTTTC | |
| | TTATGGACCTTGAAGGAAAA | |
| | CAGGGTAATTTCAAAAATCT | |
| | TAGGGAATTTGTGTTTAAGA | |
| | ATATTGATGGTTATTTTAAA | |
| | ATATATTCTAAGCACACGCC | |
| | TATTAATTTAGTGCGTGATC | |
| | TCCCTCAGGGTTTTTCGGCT | |
| | TTAGAACCATTGGTAGATTT | |
| | GCCAATAGGTATTAACATCA | |
| | CTAGGTTTCAAACTTTACTT | |
| | GCTTTACATAGAAGTTATTT | |
| | GACTCCTGGTGATTCTTCTT | |
| | CAGGTTGGACAGCTGGTGCT | |
| | GCAGCTTATTATGTGGGTTA | |
| | TCTTCAACCTAGGACTTTTC | |
| | TATTAAAATATAATGAAAAT | |
| | GGAACCATTACAGATGCTGT | |
| | AGACTGTGCACTTGACCCTC | |
| | TCTCAGAAACAAAGTGTACG | |
| | TTGAAATCCTTCACTGTAGA | |
| | AAAAGGAATCTATCAAACTT | |
| | CTAACTTTAGAGTCCAACCA | |
| | ACAGAATCTATTGTTAGATT | |
| | TCCTAATATTACAAACTTGT | |
| | GCCCTTTTGGTGAAGTTTTT | |
| | AACGCCACCAGATTTGCATC | |
| | TGTTTATGCTTGGAACAGGA | |
| | AGAGAATCAGCAACTGTGTT | |
| | GCTGATTATTCTGTCCTATA | |
| | TAATTCCGCATCATTTTCCA | |
| | CTTTTAAGTGTTATGGAGTG | |
| | TCTCCTACTAAATTAAATGA | |
| | TCTCTGCTTTACTAATGTCT | |
| | ATGCAGATTCATTTGTAATT | |
| | AGAGGTGATGAAGTCAGACA | |
| | AATCGCTCCAGGGCAAACTG | |
| | GAAAGATTGCTGATTATAAT | |
| | TATAAATTACCAGATGATTT | |
| | TACAGGCTGCGTTATAGCTT | |
| | GGAATTCTAACAATCTTGAT | |
| | TCTAAGGTTGGTGGTAATTA | |
| | TAATTACCTGTATAGATTGT | |
| | TTAGGAAGTCTAATCTCAAA | |
| | CCTTTTGAGAGAGATATTTC | |
| | AACTGAAATCTATCAGGCCG | |
| | GTAGCACACCTTGTAATGGT | |
| | GTTGAAGGTTTTAATTGTTA | |
| | CTTTCCTTTACAATCATATG | |
| | GTTTCCAACCCACTAATGGT | |
| | GTTGGTTACCAACCATACAG | |
| | AGTAGTAGTACTTTCTTTTG | |
| | AACTTCTACATGCACCAGCA | |
| | ACTGTTTGTGGACCTAAAAA | |
| | GTCTACTAATTTGGTTAAAA | |
| | ACAAATGTGTCAATTTCAAC | |
| | TTCAATGGTTTAACAGGCAC | |
| | AGGTGTTCTTACTGAGTCTA | |
| | ACAAAAAGTTTCTGCCTTTC | |
| | CAACAATTTGGCAGAGACAT | |
| | TGCTGACACTACTGATGCTG | |
| | TCCGTGATCCACAGACACTT | |
| | GAGATTCTTGACATTACACC | |
| | ATGTTCTTTTGGTGGTGTCA | |
| | GTGTTATAACACCAGGAACA | |
| | AATACTTCTAACCAGGTTGC | |
| | TGTTCTTTATCAGGATGTTA | |
| | ACTGCACAGAAGTCCCTGTT | |
| | GCTATTCATGCAGATCAACT | |
| | TACTCCTACTTGGCGTGTTT | |
| | ATTCTACAGGTTCTAATGTT | |
| | TTTCAAACACGTGCAGGCTG | |
| | TTTAATAGGGGCTGAACATG | |
| | TCAACAACTCATATGAGTGT | |
| | GACATACCCATTGGTGCAGG | |
| | TATATGCGCTAGTTATCAGA | |
| | CTCAGACTAATTCTCCTCGG | |
| | CGGGCACGTAGTGTAGCTAG | |
| | TCAATCCATCATTGCCTACA | |
| | CTATGTCACTTGGTGCAGAA | |
| | AATTCAGTTGCTTACTCTAA | |
| | TAACTCTATTGCCATACCCA | |
| | CAAATTTTACTATTAGTGTT | |
| | ACCACAGAAATTCTACCAGT | |
| | GTCTATGACCAAGACATCAG | |
| | TAGATTGTACAATGTACATT | |
| | TGTGGTGATTCAACTGAATG | |
| | CAGCAATCTTTTGTTGCAAT | |
| | ATGGCAGTTTTTGTACACAA | |
| | TTAAACCGTGCTTTAACTGG | |
| | AATAGCTGTTGAACAAGACA | |
| | AAAACACCCAAGAAGTTTTT | |
| | GCACAAGTCAAACAAATTTA | |
| | CAAAACACCACCAATTAAAG | |
| | ATTTTGGTGGTTTTAATTTT | |
| | TCACAAATATTACCAGATCC | |
| | ATCAAAACCAAGCAAGAGGT | |
| | CATTTATTGAAGATCTACTT | |
| | TTCAACAAAGTGACACTTGC | |
| | AGATGCTGGCTTCATCAAAC | |
| | AATATGGTGATTGCCTTGGT | |
| | GATATTGCTGCTAGAGACCT | |
| | CATTTGTGCACAAAAGTTTA | |
| | ACGGCCTTACTGTTTTGCCA | |
| | CCTTTGCTCACAGATGAAAT | |
| | GATTGCTCAATACACTTCTG | |
| | CACTGTTAGCGGGTACAATC | |
| | ACTTCTGGTTGGACCTTTGG | |
| | TGCAGGTGCTGCATTACAAA | |
| | TACCATTTGCTATGCAAATG | |
| | GCTTATAGGTTTAATGGTAT | |
| | TGGAGTTACACAGAATGTTC | |
| | TCTATGAGAACCAAAAATTG | |
| | ATTGCCAACCAATTTAATAG | |
| | TGCTATTGGCAAAATTCAAG | |
| | ACTCACTTTCTTCCACAGCA | |
| | AGTGCACTTGGAAAACTTCA | |
| | AGATGTGGTCAACCAAAATG | |
| | CACAAGCTTTAAACACGCTT | |
| | GTTAAACAACTTAGCTCCAA | |
| | TTTTGGTGCAATTTCAAGTG | |
| | TTTTAAATGATATCCTTTCA | |
| | CGTCTTGACAAAGTTGAGGC | |
| | TGAAGTGCAAATTGATAGGT | |
| | TGATCACAGGCAGACTTCAA | |
| | AGTTTGCAGACATATGTGAC | |
| | TCAACAATTAATTAGAGCTG | |
| | CAGAAATCAGAGCTTCTGCT | |
| | AATCTTGCTGCTACTAAAAT | |
| | GTCAGAGTGTGTACTTGGAC | |
| | AATCAAAAAGAGTTGATTTT | |
| | TGTGGAAAGGGCTATCATCT | |
| | TATGTCCTTCCCTCAGTCAG | |
| | CACCTCATGGTGTAGTCTTC | |
| | TTGCATGTGACTTATGTCCC | |
| | TGCACAAGAAAAGAACTTCA | |
| | CAACTGCTCCTGCCATTTGT | |
| | CATGATGGAAAAGCACACTT | |
| | TCCTCGTGAAGGTGTCTTTG | |
| | TTTCAAATGGCACACACTGG | |
| | TTTGTAACACAAAGGAATTT | |
| | TTATGAACCACAAATCATTA | |
| | CTACAGACAACACATTTGTG | |
| | TCTGGTAACTGTGATGTTGT | |
| | AATAGGAATTGTCAACAACA | |
| | CAGTTTATGATCCTTTGCAA | |
| | CCTGAATTAGACTCATTCAA | |
| | GGAGGAGTTAGATAAATATT | |
| | TTAAGAATCATACATCACCA | |
| | GATGTTGATTTAGGTGACAT | |
| | CTCTGGCATTAATGCTTCAG | |
| | TTGTAAACATTCAAAAAGAA | |
| | ATTGACCGCCTCAATGAGGT | |
| | TGCCAAGAATTTAAATGAAT | |
| | CTCTCATCGATCTCCAAGAA | |
| | CTTGGAAAGTATGAGCAGTA | |
| | TATAAAATGGCCATGGTACA | |
| | TTTGGCTAGGTTTTATAGCT | |
| | GGCTTGATTGCCATAGTAAT | |
| | GGTGACAATTATGCTTTGCT | |
| | GTATGACCAGTTGCTGTAGT | |
| | TGTCTCAAGGGCTGTTGTTC | |
| | TTGTGGATCCTGCTGCAAAT | |
| | TTGATGAAGACGACTCTGAG | |
| | CCAGTGCTCAAAGGAGTCAA | |
| | ATTACATTACACATAAACGA | |
| | ACTTATGGATTTGTTTATGA | |
| | GAATCTTCACAATTGGAACT | |
| | GTAACTTTGAAGCAAGGTGA | |
| | AATCAAGGATGCTACTCCTT | |
| | CAGATTTTGTTCGCGCTACT | |
| | GCAACGATACCGATACAAGC | |
| | CTCACTCCCTTTCGGATGGC | |
| | TTATTGTTGGCGTTGCACTT | |
| | CTTGCTGTTTTTCAGAGCGC | |
| | TTCCAAAATCATAACCCTCA | |
| | AAAAGAGATGGCAACTAGCA | |
| | CTCTCCAAGGGTGTTCACTT | |
| | TGTTTGCAACTTGCTGTTGT | |
| | TGTTTGTAACAGTTTACTCA | |
| | CACCTTTTGCTCGTTGCTGC | |
| | TGGCCTTGAAGCCCCTTTTC | |
| | TCTATCTTTATGCTTTAGTC | |
| | TACTTCTTGCAGAGTATAAA | |
| | CTTTGTAAGAATAATAATGA | |
| | GGCTTTGGCTTTGCTGGAAA | |
| | TGCCGTTCCAAAAACCCATT | |
| | ACTTTATGATGCCAACTATT | |
| | TTCTTTGCTGGCATACTAAT | |
| | TGTTACGACTATTGTATACC | |
| | TTACAATAGTGTAACTTCTT | |
| | CAATTGTCATTACTTCAGGT | |
| | GATGGCACAACAAGTCCTAT | |
| | TTCTGAACATGACTACCAGA | |
| | TTGGTGGT | |
| 2 | MFIFLLFLTLTSGSDLDRCT | SARS- |
| | TFDDVQAPNYTQHTSSMRGV | CoV-2 |
| | YYPDEIFRSDTLYLTQDLFL | Spike |
| | PFYSNVTGFHTINHTFGNPV | Protein |
| | IPFKDGIYFAATEKSNVVRG | (S Protein) |
| | WVFGSTMNNKSQSVIIINNS | Amino |
| | TNVVIRACNFELCDNPFFAV | Acid |
| | SKPMGTQTHTMIFDNAFNCT | Sequence, |
| | FEYISDAFSLDVSEKSGNFK | GenBank |
| | HLREFVFKNKDGFLYVYKGY | Accession |
| | QPIDVVRDLPSGFNTLKPIF | P59594 |
| | KLPLGINITNFRAILTAFSP | |
| | AQDIWGTSAAAYFVGYLKPT | |
| | TFMLKYDENGTITDAVDCSQ | |
| | NPLAELKCSVKSFEIDKGIY | |
| | QTSNFRVVPSGDVVRFPNIT | |
| | NLCPFGEVENATKFPSVYAW | |
| | ERKKISNCVADYSVLYNSTF | |
| | FSTFKCYGVSATKLNDLCFS | |
| | NVYADSFVVKGDDVRQIAPG | |
| | QTGVIADYNYKLPDDFMGCV | |
| | LAWNTRNIDATSTGNYNYKY | |
| | RYLRHGKLRPFERDISNVPF | |
| | SPDGKPCTPPALNCYWPLND | |
| | YGFYTTTGIGYQPYRVVVLS | |
| | FELLNAPATVCGPKLSTDLI | |
| | KNQCVNFNFNGLTGTGVLTP | |
| | SSKRFQPFQQFGRDVSDFTD | |
| | SVRDPKTSEILDISPCSFGG | |
| | VSVITPGTNASSEVAVLYQD | |
| | VNCTDVSTAIHADQLTPAWR | |
| | IYSTGNNVFQTQAGCLIGAE | |
| | HVDTSYECDIPIGAGICASY | |
| | HTVSLLRSTSQKSIVAYTMS | |
| | LGADSSIAYSNNTIAIPTNF | |
| | SISITTEVMPVSMAKTSVDC | |
| | NMYICGDSTECANLLLQYGS | |
| | FCTQLNRALSGIAAEQDRNT | |
| | REVFAQVKQMYKTPTLKYFG | |
| | GFNFSQILPDPLKPTKRSFI | |
| | EDLLFNKVTLADAGFMKQYG | |
| | ECLGDINARDLICAQKFNGL | |
| | TVLPPLLTDDMIAAYTAALV | |
| | SGTATAGWTFGAGAALQIPF | |
| | AMQMAYRFNGIGVTQNVLYE | |
| | NQKQIANQFNKAISQIQESL | |
| | TTTSTALGKLQDVVNQNAQA | |
| | LNTLVKQLSSNFGAISSVLN | |
| | DILSRLDKVEAEVQIDRLIT | |
| | GRLQSLQTYVTQQLIRAAEI | |
| | RASANLAATKMSECVLGQSK | |
| | RVDFCGKGYHLMSFPQAAPH | |
| | GVVFLHVTYVPSQERNFTTA | |
| | PAICHEGKAYFPREGVFVFN | |
| | GTSWFITQRNFFSPQIITTD | |
| | NTFVSGNCDVVIGIINNTVY | |
| | DPLQPELDSFKEELDKYFKN | |
| | HTSPDVDLGDISGINASVVN | |
| | IQKEIDRLNEVAKNLNESLI | |
| | DLQELGKYEQYIKWPWYVWL | |
| | GFIAGLIAIVMVTILLCCMT | |
| | SCCSCLKGACSCGSCCKFDE | |
| | DDSEPVLKGVKLHYT | |
| 3 | MSDNGPQSNQRSAPRITFGG | SARS- |
| | PTDSTDNNQNGGRNGARPKQ | CoV-2 |
| | RRPQGLPNNTASWFTALTQH | Nucleo- |
| | GKEELRFPRGQGVPINTNSG | capsid |
| | PDDQIGYYRRATRRVRGGDG | Protein |
| | KMKELSPRWYFYYLGTGPEA | (N |
| | SLPYGANKEGIVWVATEGAL | Protein) |
| | NTPKDHIGTRNPNNNAATVL | Amino |
| | QLPQGTTLPKGFYAEGSRGG | Acid |
| | SQASSRSSSRSRGNSRNSTP | Sequence, |
| | GSSRGNSPARMASGGGETAL | GenBank |
| | ALLLLDRLNQLESKVSGKGQ | Accession |
| | QQQGQTVTKKSAAEASKKPR | P59595 |
| | QKRTATKQYNVTQAFGRRGP | |
| | EQTQGNFGDQDLIRQGTDYK | |
| | HWPQIAQFAPSASAFFGMSR | |
| | IGMEVTPSGTWLTYHGAIKL | |
| | DDKDPQFKDNVILLNKHIDA | |
| | YKTFPPTEPKKDKKKKTDEA | |
| | QPLPQRQKKQPTVTLLPAAD | |
| | MDDFSRQLQNSMSGASADST | |
| | QA | |
| 4 | MADNGTITVEELKQLLEQWN | SARS- |
| | LVIGFLFLAWIMLLQFAYSN | CoV-2 |
| | RNRFLYIIKLVFLWLLWPVT | Membrane |
| | LACFVLAAVYRINWVTGGIA | Protein |
| | IAMACIVGLMWLSYFVASFR | (M |
| | LFARTRSMWSFNPETNILLN | Protein) |
| | VPLRGTIVTRPLMESELVIG | Amino |
| | AVIIRGHLRMAGHSLGRCDI | Acid |
| | KDLPKEITVATSRTLSYYKL | Sequence, |
| | GASQRVGTDSGFAAYNRYRI | GenBank |
| | GNYKLNTDHAGSNDNIALLV | Accession |
| | Q | P59596 |
| 5 | MYSFVSEETGTLIVNSVLLF | SARS- |
| | LAFVVFLLVTLAILTALRLC | CoV-2 |
| | AYCCNIVNVSLVKPTVYVYS | Envelope |
| | RVKNLNSSEGVPDLLV | Protein |
| | | (E |
| | | Protein) |
| | | Amino |
| | | Acid |
| | | Sequence, |
| | | GenBank |
| | | Accession |
| | | P59637 |
| 6 | FKNIDGYFKIYSKHTPINLV | NTD of S |
| | RDLPQGFSAL | Protein |
| 7 | PTKLNDLCFTNVYADSFVIR | RBD1 of |
| | GDEVRQIAPG | S Protein |
| 8 | IRGDEVRQIAPGQTGKIADY | RBD2 of |
| | NYKLPDDFTG | S Protein |
| 9 | RLFRKSNLKPFERDISTEIY | RBD3 of |
| | QAGSTPCNGC | S Protein |
| 10 | GVEGFNCYFPLQSYGFQPTN | RBD4 of |
| | GVGYQPYRVV | S Portein |
| 11 | MWFLTTLLLWVPVDG | 1-15 |
| | | amino |
| | | acids of |
| | | UniProtKB |
| | | FCGR |
| | | 1 HUMAN |
| 12 | MAAPGSARRPLLLLLLLLLL | LAMP1 |
| | GLMHCASA | 1-28 |
| | | amino |
| | | acids |
| 13 | MVCFRLFPVPGSGLVLVCLV | LAMP2 |
| | LGAVRSYA | 1-28 |
| | | amino |
| | | acids |
| 14 | MVVMAPRTLFLLLSGALTLT | HLA-G |
| | ETWA | 1-24 |
| | | amino |
| | | acids |
| 15 | MAISGVPVLGFFIIAVLMSA | HLA- |
| | QESWA | DRA 1- |
| | | 25_amino |
| | | acids |
| 16 | MWFLTTLLLWVPVDGQVDTT | Human |
| | KAVITLQPPWVSVFQEETVT | FCGR1A |
| | LHCEVLHLPGSSSTQWFLNG | _amino |
| | TATQTSTPSYRITSASVNDS | acids; |
| | GEYRCQRGLSGRSDPIQLEI | NP_0005 |
| | HRGWLLLQVSSRVFTEGEPL | 57.1. |
| | ALRCHAWKDKLVYNVLYYRN | (Underline |
| | GKAFKFFHWNSNLTILKTNI | & bold: |
| | SHNGTYHCSGMGKHRYTSAG | signal |
| | ISVTVKELFPAPVLNASVTS | peptide) |
| | PLLEGNLVTLSCETKLLLQR | |
| | PGLQLYFSFYMGSKTLRGRN | |
| | TSSEYQILTARREDSGLYWC | |
| | EAATEDGNVLKRSPELELQV | |
| | LGLQLPTPVWFHVLFYLAVG | |
| | IMFLVNTVLWVTIRKELKRK | |
| | KKWDLEISLDSGHEKKVISS | |
| | LQEDRHLEEELKCQEQKEEQ | |
| | LQEGVHRKEPQGAT | |

Signal peptides (usually 16-30 amino acids long) which exist in front of the N-terminal of many precursor proteins can guide those proteins toward the intracellular destiny in the maturation and secretory trafficking process. For exosome-expressed human transmembrane protein CD64, the signal peptide can be MWFLTTLLLWVPVDG, 1-15 amino acids of UniProtKB_FCGR1_HUMAN (SEQ ID NO:11). Similar signal peptides which can guide human target protein expression on exosome surface also include LAMP1_1-28_amino acids: MAAPGSARRPLLLLLLLLLLGLMHCASA (SEQ ID NO:12); LAMP2_1-28 amino acids: MVCFRLFPVPGSGLVLVCLVLGAVRSYA (SEQ ID NO:13); HLA-G_1-24 amino acids: MVVMAPRTLFLLLSGALTLTETWA (SEQ ID NO:14); and HLA-DRA_1-25_amino acids: MAISGVPVLGFFIIAVLMSAQESWA (SEQ ID NO:15). Introduction of such signal peptide motifs both can protect the synthesized protein from being consumed in the cytosol and increases the protein expression on the exosome surface.

In some cases, the composition comprising the extracellular vesicle can further comprise an immune modulator or an adjuvant to enhance immune response triggered by the viral mimic peptide contacting the immune cell. Exemplary immune modulator includes pathogen-associated molecular patterns (PAMPs) molecule, damage-associated molecular patterns (DAMPs) molecule, Toll-like receptor agonist, STING agonist, RIG-I agonist, tumor necrosis factor (TNF) ligand, or cytokine (such as IL-2, IL-12, 1L-15 or IL21). Exemplary adjuvant includes inorganic compounds (e.g. alum, aluminum hydroxide, aluminum phosphate, or calcium phosphate hydroxide), mineral oil, paraffin oil, peanut oil, bacterial products such as inactivated Bordetella pertussis, nonbacterial organics like squalene, plant saponins, Freund's complete adjuvant, or Freund's incomplete adjuvant.

Described herein, in some instances, are methods of vaccinating a subject in need thereof, said method comprising administering a therapeutically effective amount of a pharmaceutical composition comprising the extracellular vesicle comprising the viral mimic peptide. In some cases, the pharmaceutical composition comprising the viral mimic peptide is administered to the subject at least once per day, at least once per week, at least once per month, at least once per year, or at least once per a period of time that is longer than one year.

Once neutralizing antibody against the viral mimic peptide is observed in the subject, the administration can be stopped. Alternatively, a maintenance dose or a booster dose of the pharmaceutical composition comprising the extracellular vesicle comprising the viral mimic peptide is administered if necessary. Subsequently, the dosage or the frequency of administration, or both, can be reduced, as a function of the level of neutralizing antibody detected in the subject.

Described herein, in some cases, are methods of producing the extracellular vesicle comprising the viral mimic peptide. In some cases, the method comprises introducing at least one heterologous polynucleotide into an extracellular vesicle donor cell. In some cases, the at least one heterologous polynucleotide is a vector (e.g., plasmid). In some instances, the at least one heterologous polynucleotide introduced into the extracellular vesicles cells encodes at least one adapter polypeptide described herein. In some cases, the at least one heterologous polynucleotide encodes at least one targeting domain. In some instances, the at least one heterologous polynucleotide encodes the viral mimic peptide.

In some cases, the heterologous polynucleotide can be introduced into the cell via the use of expression vectors. In the context of an expression vector, the vector can be readily introduced into the cell described herein by any method in the art. For example, the expression vector can be transferred into the cell by biological, chemical, or physical methods of transfection described here. In some cases, the heterologous polynucleotide is transfected into the extracellular donor cell by nanoelectroporation as described herein.

### Pharmaceutical Compositions

In some cases, the extracellular vesicles can be formulated into pharmaceutical composition. In some cases, the pharmaceutical composition comprising the extracellular vesicle comprises at least one pharmaceutically acceptable excipient. In some cases, the pharmaceutical composition comprising the extracellular vesicle can be administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration. In some cases, the pharmaceutical composition and formulations described herein are administered to a subject by intravenous, subcutaneous, and intramuscular administration. In some cases, the pharmaceutical composition and formulations described herein are administered to a subject by intravenous administration. In some cases, the pharmaceutical composition and formulations described herein are administered to a subject by administration. In some cases, the pharmaceutical composition and formulations described herein are administered to a subject by intramuscular administration.

### Kits/Article of Manufactures

Disclosed herein, in certain cases, are kits and articles of manufacture for use with one or more methods and compositions described herein. Also described herein are systems of manufacturing the extracellular vesicles described herein. In some cases, the system comprises components to nanoelectroporate extracellular vesicle donor cell to stimulate the production and secretion of extracellular vesicles comprising the adapter polypeptides and the therapeutic described herein.

In some cases, the kit can include a carrier, package, or container that is compartmentalized to receive one or more containers such as vials, tubes, and the like, each of the container (s) comprising one of the separate elements to be used in the methods described herein. Suitable containers include, for example, bottles, vials, syringes, and test tubes. In some cases, the containers can be formed from a variety of materials such as glass or plastic. A kit typically includes labels listing contents and/or instructions for use, and package inserts with instructions for use. A set of instructions can also be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain cases, the extracellular vesicle comprising the adapter polypeptide and the therapeutic can be presented in a pack or dispenser device which contains one or more unit dosage forms containing a compound provided herein. In certain cases, the extracellular vesicle comprising the adapter polypeptide complexed with any one of the antibodies described herein and the therapeutic can be presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, the extracellular vesicle comprising the adapter polypeptide and the therapeutic containing provided herein formulated in a compatible pharmaceutical carrier are also prepared, placed in an appropriate container, and labeled for treatment of an indicated condition. In some cases, the kit comprises articles of manufacture that are useful for developing vaccines, therapeutics, adoptive therapies, and methods of treatment described herein.

### EXAMPLES

The following illustrative examples are representative of aspects of the stimulation, systems, and methods described herein and are not meant to be limiting in any way.

### Example 1. Design and Testing of THP-CD64 Plasmid DNA

A new platform of antibody and peptide linked extracellular vesicles (“EVs”), particularly exosomes, was established by transfecting donor cells with plasmid DNA expressing CD64, CD64-peptide, or other Fc receptors on the EV and exosome surface. CD64 is also known as Fc-gamma receptor 1 (FcγR1), which binds to the hinge of the Fc region of IgG1 and IgG3 by its extracellular D1 and D2 domains with high affinity with a dissociation constant, K_{d}, at nanomolar (nM) levels. Donor cells were also co-transfected with plasmid DNAs in order to add endogenous RNAs and proteins into the EVs and exosomes to function as therapeutics. These therapeutic EVs (tEVs) and exosomes (tExos) served as targeted drug delivery vehicles to cancer cells and tumors, non-cancer lesions, and damaged tissues. They were also designed for vaccine development and other medical treatments.

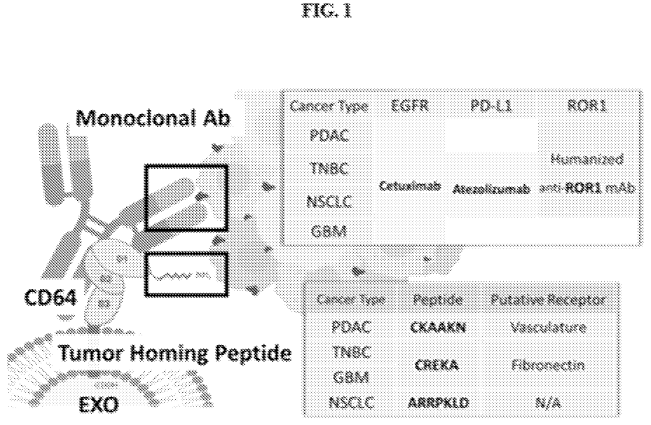

Tumor homing peptides (“THPs”) designed to encode a FLAG tag, CKAAKN (CK), CREKA (CR), or ARRPKLD (AR) were added to the N-terminus of CD64, and various humanized monoclonal antibodies (mAbs) bound onto the D1 and D2 domains of CD64 to achieve the dual targeting ability as shown in FIG. 1. The extracellular vesicles (“EVs”) with CD64 or THP-CD64 were generated by transfection of donor cells with human CD64 plasmid DNA or human THP-CD64 plasmid DNA expressing either human CD64 or human THP-CD64 on the surface of EVs (such as exosomes) secreted from the transfected donor cells. CD64 served as a biological anchor for binding a humanized monoclonal antibody (“hmAb”). The extracellular D1 and D2 domains of human CD64 bound to the lower hinge region of Fc in human IgG1 with high affinity, e.g., a dissociation constant (K_{d}) at the nanomolar level. In addition to the specific recognition ability of the bound hmAb, targeting by small tumor homing peptides (THPs) was also engineered to the N-terminal of CD64. Dual targeting of both the hmAb and the THP on the EV (or exosome) surface enhanced targeting of the EV (or exosome) delivery to tumors and other lesions in vivo.

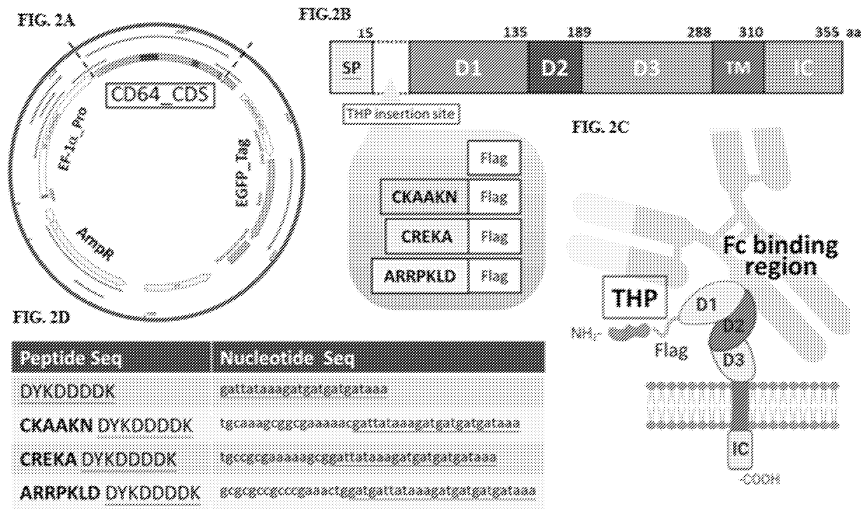

Plasmids were constructed with a vector containing genes for Ampicillin resistance (AmpR) and the EGFR marker for transformation and transfection, respectively. The functional CD64 was encoded by the coding sequence of CD64 (CD64_CDS) driven by the EF1-promoter (FIG. 2A). The CD64_CDS (355 amino acids) consisted of (i) signal peptide (SP), (ii) extracellular (D1, D2, and D3) domains, (iii) transmembrane (TM) domain, and (iv) intracellular (IC) domain, and the THPs were inserted into the gap of signal peptide and extracellular D1 to be expressed on the N-terminus of CD64 (FIG. 2B). THPs were connected by a Flag (DYKDDDK) linker to the N-terminus of extracellular D1, limiting the conformational block on the Fc binding region at D1-D2 hinge of CD64 (FIG. 2C). The peptide and nucleotide sequences of the THPs: Flag_control, CKAAKN (CK), CREKA (CR), and ARRPKLD (AR) are listed in FIG. 2D.

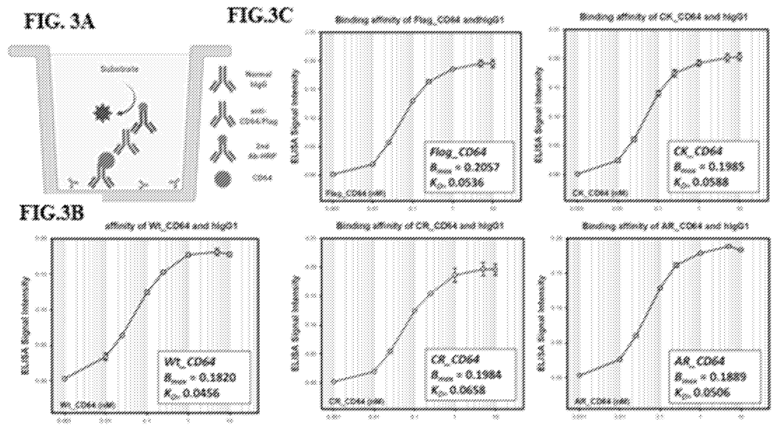

To test whether tumor homing peptides (“THPs”) linked to the N-terminal of CD64 would change the interaction of CD64 with human immunoglobulin G (hIgG), engineered CD64 proteins with different THPs were purified and reacted with immobilized hIgG (coated onto a 96-well plate) in order to perform a sandwich Enzyme-Linked Immunosorbent Assay (ELISA) (FIG. 3A). The bound CD64 proteins with different THPs were reacted with anti-CD64/Flag and 2nd HRP antibodies, followed with ELISA substrates (Tetramethylbenzidine). The absorbance at 450 nm revealed the concentration of titration with engineered CD64 proteins. The binding titration curves were fitted to the monovalent modeling between CD64 and hIgG to determine the dissociation constant K_{d} (O.D.=[Bₘₐₓ×Con]/[K_{d}+Con] where O.D. was the optical density of absorbance, Bₘₐₓ was the maximum of binding in the unit of O.D, and Con was concentration). The K_{d} of hIgG and recombinant wild-type CD64 (wt_CD64) was first measured as a reference (K_{d}=0.0456 nM, FIG. 3B). The affinity index K_{d} of different engineered THP-CD64 proteins with hIgG was determined as Flag-CD64 (K_{d}=0.0536 nM), CK-CD64 (K_{d}=0.0588 nM), CR-CD64 (K_{d}=0.0658 nM), and AR-CD64 (K_{d}=0.0506 nM) (FIG. 3C). These results indicated that the engineered CD64 with different THPs (Flag, CK, CR, or AR) did not affect the high binding affinity to mAb in nM-levels, as compared to wt CD64.

### Example 2. Nanochannel Electroporation (NEP) Triggered EV and Exosome Release from Transfected Cells

THP-CD64 containing EVs and exosomes were produced by using a nanochannel electroporation (“NEP”) system. The donor cells were cultured on the chip surface. After culturing for one day, plasmids pre-loaded in the cargo chamber were injected into individual cells via nanochannels using a 25˜250 V electric field (depending on cell type and source) with 10 pulses at 10 ms per pulse at a 0.1 s interval. After cell transfection, released EVs (including exosomes) which contained functional RNAs and surface receptors were purified from collected culture medium via centrifuge first to remove cells and large cell debris, followed by tangential flow filtration (TFF).

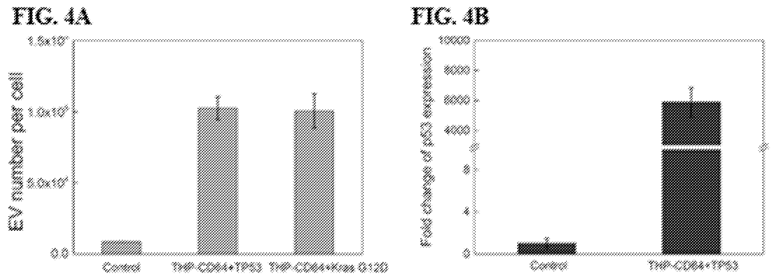

FIG. 4 shows EV number and endogenous RNA content from NEP transfected mouse embryonic fibroblasts (MEFs) with THP-CD64 and therapeutic RNA plasmids. After 24 hours of NEP treatment, cell culture medium was collected for purification and recovery of EVs through centrifuge and TFF. After purification, the engineered EVs were further purified into a high concentration with a volume about 200 μL using a spinning column. As shown in FIG. 4A, EV number of both human THP-CD64+human TP53 group and human THP-CD64+shKRAS G12D mutation group showed around 10-fold increase after NEP treatment when compared with the control group (i.e. without NEP treatment). RT-qPCR of TP53 mRNA expression in FIG. 4B revealed that EVs produced by NEP contained a high quantity of transcribed mRNAs comparing to the control group without NEP treatment, an estimated ˜6,000 fold increase based on a Ct value of 27.5 vs. undetermined at 40.

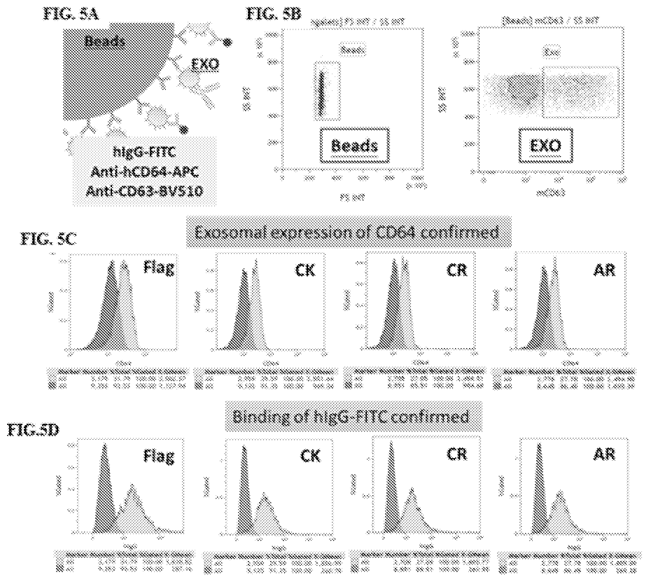

Purified exosomes with engineered THP-CD64 were captured by latex beads and incubated with anti-CD64-APC, anti-CD63-BV510, and FITC-conjugated hIgG for flow cytometry assay (FIG. 5A). Profiling of surface expression followed the standard protocol to gate the singlet bead and CD63⁺ exosome population in order to determine the mean fluorescence intensity (MFI) of CD64 expression and hIgG affiliation as shown in FIG. 5B. Surface co-expression of CD64 within the CD63⁺ exosomal population was determined by MFI of FITC and confirmed the exosomal expression of engineered CD64 with either Flag, CK, CR or AR THP as shown in FIG. 5C. Surface co-expression of hIgG and CD64 within the CD63⁺ exosomal population was determined by MFI of FITC and confirmed the high binding affinity of hIgG on exosomes expressing CD64 with either Flag, CK, CR or AR THP as shown in FIG. 5D.

### Example 3. Uptake of THP-CD64 Containing Exosomes with or without hmAb in PANC-1 Spheroids

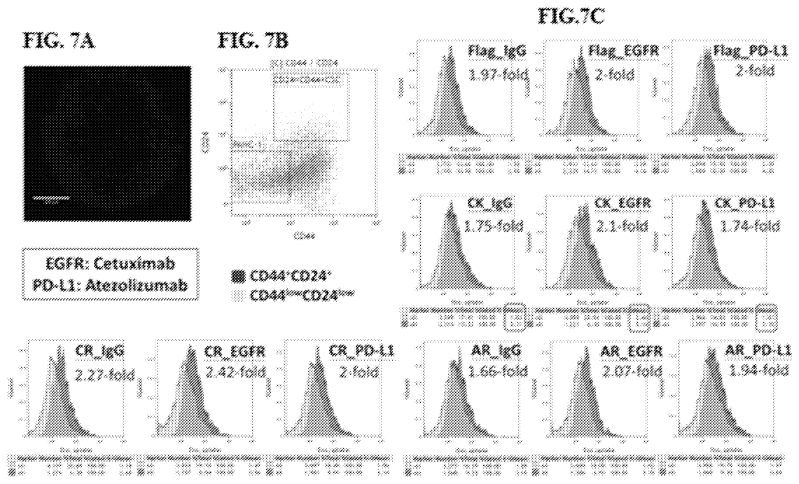

Spheroids of a pancreatic cancer cell line, PANC-1 were formed by hanging drop method using cellulose and collagen type 1 and cultured for a week to reach a diameter of ˜500-600 μm as shown in FIG. 7A. Pancreatic cancer stem cells (CSCs) are commonly defined by their surface expression of CD44 and CD24. Spheroids exceeding 400 μm in diameter develop a hypoxic core, and this hypoxic microenvironment activates survival signaling pathways and reprograming to maintain cell viability. As PANC-1 cells were cultured stably in spheroids, the CD44⁺CD24⁺ population gradually increased.

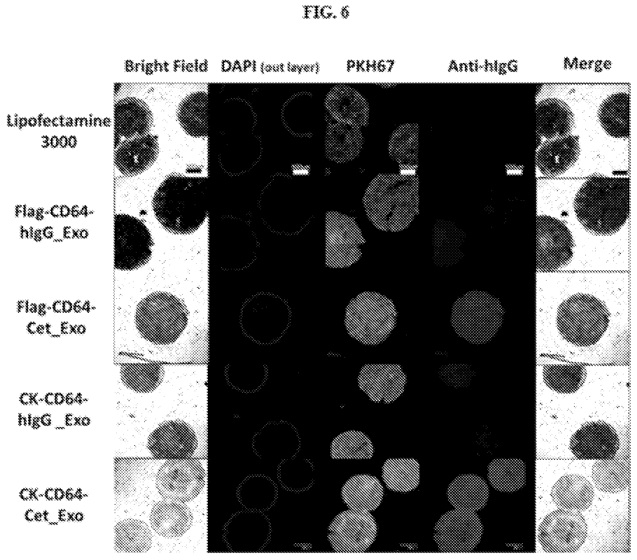

The purified EVs released from mouse embryonic fibroblast (MEF) cells after transfection of either Flag-CD64 or CK-CD64 plasmid DNA (CK-CD64) were formulated with either humanized anti-EGFR mAb (Cetuximab) or hIgG. The cancer spheroids formed from the human pancreatic cancer cell line PANC-1 were treated with PKH67 (green)-labeled liposome (lipofectamine 3000) or various EVs for 24 h, and subsequently processed by fixation, permeation, and staining with anti-hIgG-TRITC (red) and DAPI (blue). The cross section of cancer spheroids was imaged under confocal microscopy. Cancer spheroid treatment with various EVs all showed better spheroid uptake than the commercial lipofectamine 3000 based on fluorescence intensity and distribution. Among various EVs, the dual targeting exosome (CK-CD64-Cet_Exo) revealed the highest spheroid uptake as shown in FIG. 6.

To further evaluate cellular uptake of various THP-CD64 EVs with or without human monoclonal antibodies (hmAb), the treated spheroids were disassembled into single-cell suspension to identify the subpopulations by CD24 and CD44 expression using flow cytometry as shown in FIG. 7B. The mean fluorescence intensity of PKH67 measured in CD24^{low}CD44^{low} or CD24⁺CD44⁺ subpopulations represented their EV uptake. The engineered EVs containing Flag-CD64, CK-CD64, CR-CD64, or AR-CD64 with humanized antibody affiliation (Cetuximab: anti-EGFR, Atezolizumab: anti-PD-L1, or hIgG) all showed good cellular uptake, particularly for the CD44⁺CD24⁺ subpopulation as shown in FIG. 7C. The dual targeting EVs with anti-hEGFR (Cetuximab) and CK-CD64 provided the best cellular uptake for both PANC-1 cell subpopulations.

### Example 4. Uptake of CK-CD64/Anti-ROR1-Containing Exosomes in PANC-1 Spheroids and an Orthotopical Mouse Model

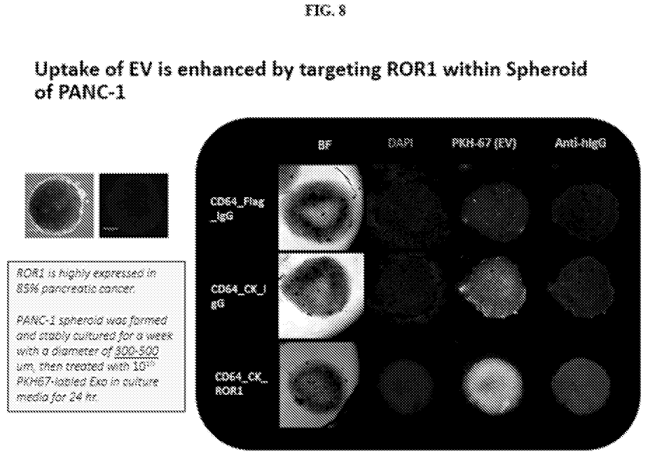

The purified EVs released from mouse embryonic fibroblast (MEF) cells after transfection of either Flag-CD64 or CK-CD64 plasmid DNA (CK-CD64) were formulated with humanized anti-ROR1. The cancer spheroids formed from the human pancreatic cancer cell line PANC-1 were treated with PKH67 (green)-labeled liposome (lipofectamine 3000) or various EVs for 24 h, and subsequently processed by fixation, permeation, and staining with anti-hIgG-TRITC (red) and DAPI (blue). The cross section of cancer spheroids was imaged under confocal microscopy. Among various EVs, the dual targeting exosome (CK-CD64-ROR1_Exo) revealed the highest spheroid uptake as shown in FIG. 8.

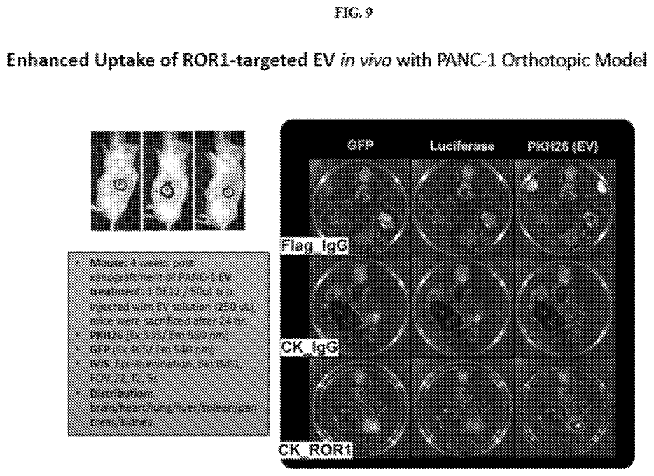

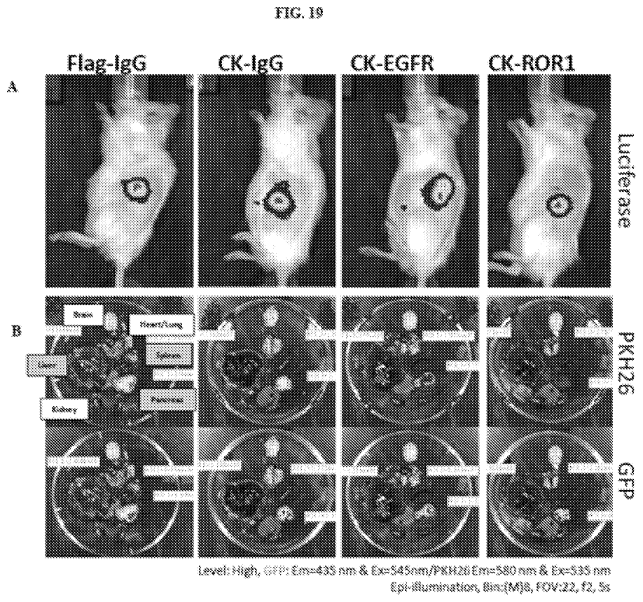

The PANC-1 cells were transduced with GP and luciferase for tracing their localization in vivo. Various EVs were tail vein injected into NOD scid gamma (NSG) mice 4 weeks after xenografted orthotopically with PANC-1 cancer cells. Biodistribution of EVs in brain, heart, lung, liver, spleen, kidney and pancreas 24 hours after EV delivery was examined by PKH26 staining of the EV lipid bilayer. EV concentration was ˜. 10E12/50 uL each and donor cell was MEF. FIG. 9 and FIG. 19A-B show that CK-CD64-ROR1_Exo revealed the highest EV accumulation in the pancreas. The colocalization of PKH26, GFP, and luciferase intensity reflected the accuracy for the CK-CD64-ROR1_Exo delivered to pancreatic tumor lesions.

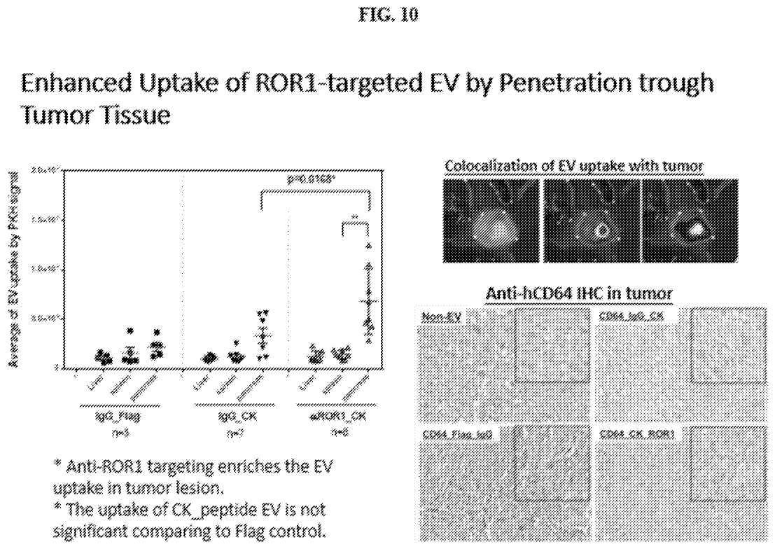

FIG. 10 compares the average of EV uptake in liver, spleen and pancreas of various EVs by PKH staining and EV distribution in tumor tissue. It is clear that CK-CD64-ROR1 Exo could provide excellent pancreas targeting and tumor tissue uptake of EVs with CK-CD64-ROR1 targeting.

### Example 5. Design Concept of Vacosomes for Vaccine Development

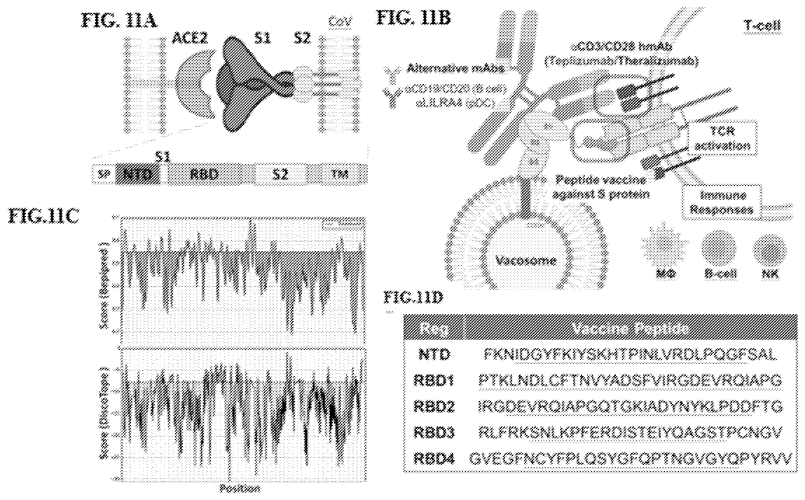

The Coronaviruses (CoV) are associated with significant risk to global health as evidenced by the various epidemics seen with several subtypes including SARS-COV-2, the causative agent of the current COVID-19 pandemic. The Spike (S) protein is an integral structural component of the viral envelope and is a strategic target for vaccine development. Exosomes that overexpress various viral S-protein fragments fused to CD64 on the exosomal surface can serve as a vaccine (designated ‘vacosome’) (FIG. 11). A strong vaccination through T-cell receptor (TCR) complex can be synergistically achieved by vaccination peptides on the N-terminal of CD64 and co-stimulation by the preloaded anti-αCD3/CD28 mAb on the hinge D1-D2 of CD64. The formation of an immunological synapse between the engineered CD64 and TCR can be confirmed by the fluorescent tag and T-cell surface markers staining using fluorescence-activated cell sorter (FACS). Similarly, the co-loading of mAb targeting antigen presenting cells (APCs) such as B cells (anti-αCD19/CD20) and dendritic cells (DCs) (anti-αLILRA4) should enhance APC-T cell responses. Five fusion S-protein fragment candidates that have high potential to serve as a vaccine peptide for COVID-19 are selected from the epitope and structural predictions. They can be expressed on vacosomes generated via NEP transfected donor cells such as human mesenchymal stem cells (MSCs) and DCs.

### Example 6. Binding Affinity Strength of Human Immunoglobins and Classical Fc Receptors

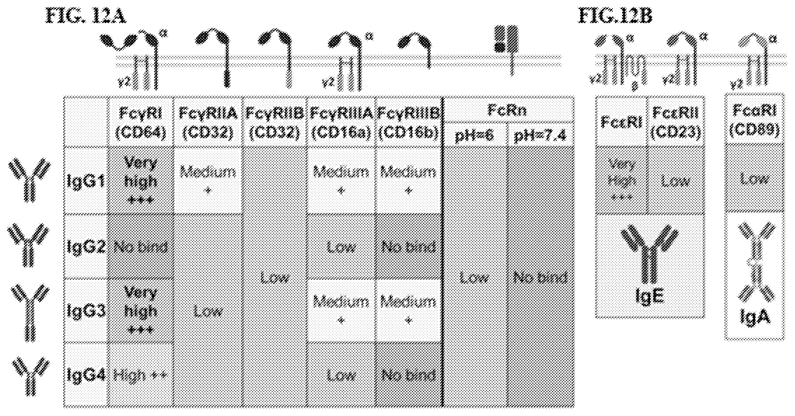

In addition to Fcγ-IgG binding, there are other human immunoglobins and Fc receptors. Fc receptors embedded in the plasma membrane contain intracellular domains or subunits that can trigger a downstream activation or suppression. IgG affinity-altering variants are highlighted in FIG. 12A with respective human Fcγ receptor members, from very high (deep orange), high (orange), medium (yellow), low (light blue), to no binding (dark blue). FcRn receptor binds to IgG subclasses under acidic conditions (e.g. pH-6) but decrease the binding ability in physiological conditions, pH=7.4. FIG. 12B shows that IgE has very high binding affinity with FcεRI receptor, but low affinity with FcεRII receptor. IgA has low binding affinity with FcαRI receptor.

### Example 7. Construction of KRAS^{G12D} siRNA/CD64 and TP53 mRNA/CD64 Targeting EVs (tEVs) Through Optimized NEP

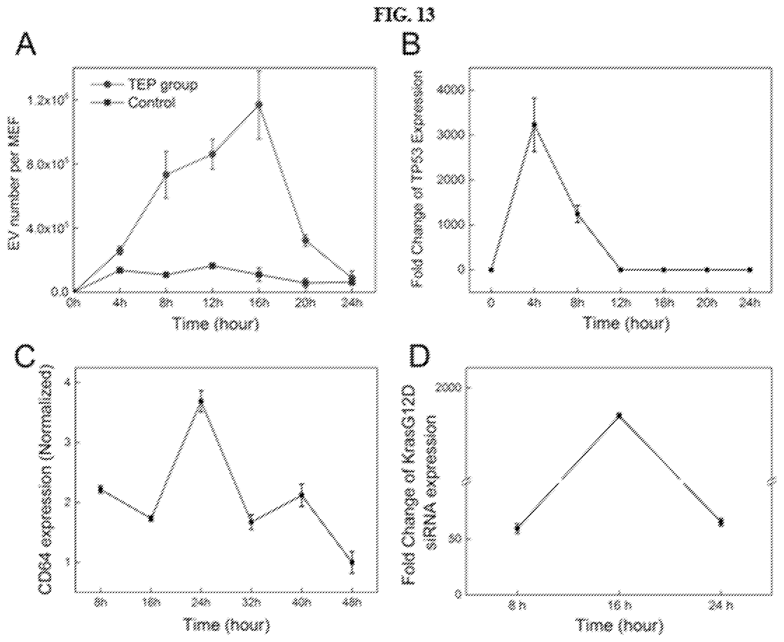

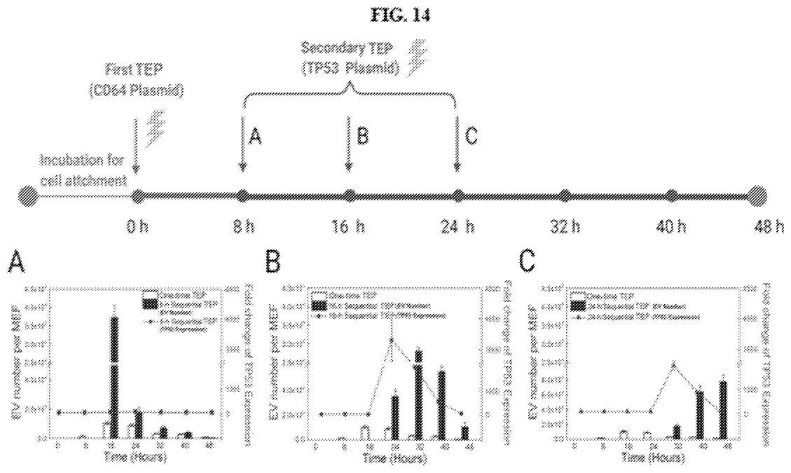

In order to design engineered EVs for an efficient targeting delivery in PDAC, the dynamics of EV release triggered by cell stimulation, and the loading profiles of therapeutics (Kras^{G12D}-specific shRNA and hTP53 mRNA) in secreted EVs as well as CD64-protein expression on EVs surface were investigated. As shown in FIG. 13, EVs secreted from MEFs significantly increased and peaked around 16 h after NEP using the 1 μm pored Transwell at 150V with ten 10 ms pulses and then dropped quickly, while the expression level of TP53-mRNA was quickly peaked around 4 and 8 h, and then the expression dropped to a very low level 12 h after NEP. The expression trend of shRNA targeting KRAS^{G12D} (FIG. 13D) is similar to that of the EV secretion profile, i.e. significantly increased and peaked around 16 h. In comparison to the EV secretion and expression of nucleotides encapsulated in EVs over time, CD64 protein on EV surface could be expressed at a high level over a long time period after NEP with a peak around 24 h after NEP. Release profiles are considered to produce EVs with both CD64 proteins and desired nucleotides in EVs. Since there is a large peak time difference between TP53 expression and EVs secretion, a sequential transfection approach of parent cells is adopted. Three types of sequential transfection designs were compared where CD64 plasmid was transfected first followed with TP53 plasmid delivery with a time lag of 8, 16 and 24 h. As shown in FIG. 14, the 8 h case showed a dramatically increased EVs secretion after the second cell stimulation, however, the TP53 mRNA expression within the EVs was very low, which could be attributed to the excessive cell stimulation within a short time period causing poor cell viability. In contrast, the 16 h and the 24 h cases resulted in much higher expressions of TP53 mRNA. Between the two, the 16 h case showed both the highest TP53 mRNA expression and very high EVs secretion. Therefore, TP53 mRNA/CD64 EVs were produced through a sequential NEP with the TP53 plasmid delivered 16 h after the first transfection of the CD64 plasmid. For KRAS^{G12D} shRNA/CD64 EVs, simultaneous delivery of both CD64 and KRAS^{G12D} shRNA plasmids in NEP worked well.

### Example 8. Characterization of the as-Prepared Targeting EVs (tEVs)

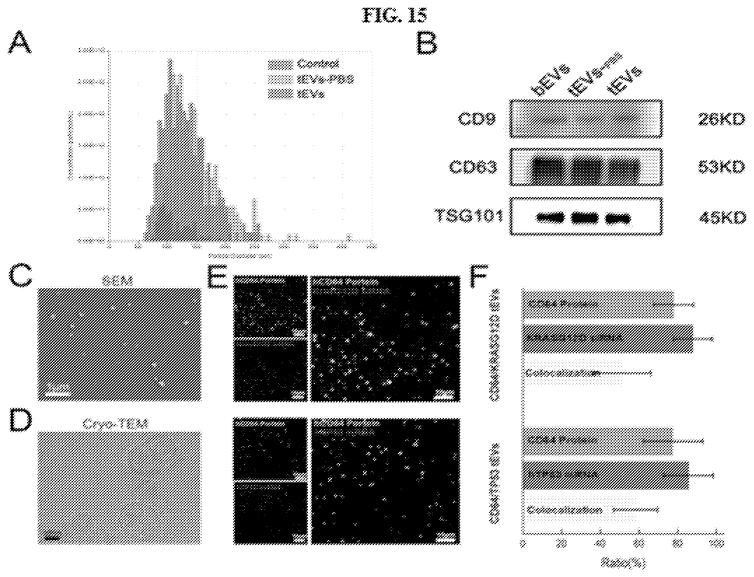

qNANO, SEM, cryo-TEM, Western Blot and immunolipoplex nanoparticle (ILN) biochip assay were conducted to observe and characterize the generated tEVs. The qNANO results (FIG. 15A) indicate that the average diameter of blank EVs was approximately 110 nm, while the engineered EVs including EVs with no cargos (PBS only) and with therapeutics (KRAS^{G12D} shRNA/CD64 EVs was taken as representative) showed a larger diameter, which suggested a large quantity of nucleotides and other biomolecules being encapsulated in EVs. FIG. 15B shows that typical exosome proteins such as CD63, CD9 and TSG101 are highly expressed in as-prepared EVs. As shown in the SEM and cryo-TEM images (FIGS. 15C-D), these engineered vesicles remained spherical. In order to confirm the encapsulated nucleic acids, surface proteins, and their colocalization within the tEVs, an ILN biochip on a TIRFM microscope was utilized for the single EV capture and detection. According to the calculated TIRFM results (FIGS. 15E-F), the colocalization ratios of encapsulated nucleotides (TP53 mRNA and Kras^{G12D} shRNA) and CD64-CK surface protein within tEVs were 51.19% and 58.31% respectively.

### Example 9. Sequential Transwell Electroporation (STEP) Protocol

Mouse embryonic fibroblast (MEF) cells, human bone marrow-derived stem cells (hBMSCs), and other cell types can be used for the therapeutic EV (tEV) production. Described herein is MEF as an example.

Mouse embryonic fibroblast (MEF) cells (obtained from Millipore Sigma) are used to generate the cell clones for therapeutic EV (tEV) production. For conventional cell culture using tissue culture flasks (Fisherbrand, Cat #FB012937), MEF cells are maintained in Dulbecco's minimal essential medium (DMEM) supplemented with 10% fetal bovine serum (FBS), 100 U/mL penicillin/streptomycin and incubated at 37° C. in 5% CO₂. MEF cells are seeded on Transwell electroporation (TEP) insert (e.g. 12 mm) with 200,000-300,000 cells per insert. When cells are grown to 80% confluency, the cells are washed 3 times with 1×DPBS and replaced in serum-free media for TEP treatment. The cells are treated by TEP using an electroporation system (Gene Pulser Xcell from Bio-Rad) with consistent electroporation parameters. For optimization, the amount of EV releasing triggered by TEP, loading profiles of therapeutics (CD64 protein, KRAS^{G12D} siRNA and hTP53 mRNA) can be screened over time through hCD64 ELISA kit (From Biocompare) and qRT-PCR. Cell culture conditioned media (CCM) are collected over time after TEP treatment and are centrifuged at 200×g for 5 minutes to remove cells and debris. The qNANO System (Izon Science) utilizes a tunable resistive pulse sensing (TRPS) method to count nanoparticle ranging from 50-330 nm in CCM solution. To measure CD64 protein, EVs are lysed by RIPA buffer (Thermo Scientific™ 89900) and hCD64 ELISA kit (From Biocompare) and then are used following the protocol of manufacture. To measure the expression level of TP53 mRNA and siRNA targeting KRAS^{G12D} within EVs, EVs are purified from cell culture medium by Total Exosome Isolation (TEI) kit (from Invitrogen). Total RNA is then isolated from an RNA Purification Kit (from Norgen). The relative expression levels of human TP53 mRNA and KRASG12D siRNA can be measured by RT-PCR and then calculated by Livak method using 2ΔΔ^{Ct} (see qRT-PCR for tEV RNAs, below). The KRAS^{G12D} shRNA/CD64 therapeutic EVs are harvested after one-time TEP treatment, while TP53 mRNA/CD64 therapeutic EVs are harvested after sequential TEP treatment. For KRAS^{G12D} shRNA/CD64 therapeutic EVs, the cells treated by one-time TEP with KRAS^{G12D} shRNA and CD64 plasmid mixture are incubated for 16 hours in serum-free media. For TP53 mRNA/CD64 therapeutic EV, the cells are first treated by TEP with CD64 plasmid and are incubated for 16 hours. Then, the cells are treated by TEP with TP53 mRNA plasmid and are incubated for another 16 hours in serum-free media. Cell culture conditioned media (CCM) is collected accordingly and is centrifuged at 200×g for 5 minutes to remove cells and debris. Centrifuge the cell removed CCM at 2000× g for 30 minutes to remove cell debris. Transfer the cell debris removed CCM to a new tube without disturbing the pellet. CCM is stored at 4° C. or is frozen and stored at −80° C. for downstream tEV isolation and characterization.

qRT-PCR for tEV RNAs. Centrifuge the cell media at 2000× g for 30 minutes to remove cells and debris. Transfer the supernatant containing the cell-free culture media to a new tube without disturbing the pellet. After NTA measurement of electroporated cell culture media, the samples are purified using total exosome isolation reagent (from cell culture media, Invitrogen, Cat #4478359). If EV concentration from NTA data is less than 2e9/mL, the samples are purified using total exosome isolation reagent (from serum, Invitrogen, Cat #) (go to step of discarding the supernatant using a pipette, below). Transfer the required volume (1 mL) of cell-free culture media to a new tube and add 0.5 volumes (0.5 mL) of the Total Exosome Isolation (TEI from cell culture media) reagent. Mix the culture media/reagent mixture well by vortexing and pipetting up and down until there is a homogenous solution. Incubate samples at 4° C. overnight. After incubation, centrifuge the samples at 10,000×g for 1 hour at 4° C. Discard the supernatant using pipet. EVs are contained in the pellet at the bottom of the tube (not visible in most cases). Resuspend the pellet in a convenient volume (100 uL) of RNase free 1×DPBS by vortexing and pipetting up and down until the solution become clear again. If the starting volume is 2 mL of culture media, transfer the resuspended solution (100 uL) back to the other tube with the pellet. For EV RNA extraction, use Plasma/Serum RNA purification Mini kit (Norgen Biotek, Cat #55000). Warm up Lysis Buffer A at 60° C. for 20 minutes and mix well until the solution become clear again if precipitates are present. Place 100 uL of TEI reagent treated sample in a 1.5 mL tube and add 300 uL of Lysis Buffer A. Mix well by vortexing for 10 seconds. Add 400 uL of 100% ethanol (200 proof). Mix well by vortexing for 10 seconds. Transfer 400 μL of the mixture from the step of placing 100 uL of TEI reagent treated sample into a Micro Spin column. Centrifuge the mixture at 3,300×g and room temperature (RT) for 2 minutes. Discard the flowthrough and reassemble the spin column with its collection tube. Repeat the steps of adding 400 uL of 100% ethanol, mixing, and transferring one more time to transfer the sample mixture into the spin column. Apply 400 uL of Wash Solution A to the column and centrifuge at 3,300×g and RT for 1 minute. Discard the flowthrough and reassemble the spin column with its collection tube. Repeat the steps of discarding the flowthrough, reassembling, and adding ethanol two more times for a total of three (3) washes. Spin the empty column at 13,000×g and RT for 2 minutes and discard the collection tube. Transfer the spin column to a fresh 1.7 mL Elution tube. Apply from 12.5 uL of Elution Solution A of to the column and let stand RT for 2 minutes. Centrifuge the spin column with the Elution tube at 400×g for 1 minute, followed by at 5,800×g for 2 minutes. For maximum recovery, transfer the eluted buffer back to the spin column and let stand at RT for 2 minutes. Centrifuge again at 400×g for 1 minute, followed by at 5,800×g for 2 minutes. To measure concentration of the extracted total RNA solution (about 10 uL), use NanoDrop 2000C spectrophotometer (Thermo Scientific). The measuring volume is recommended with 1 uL for aqueous solutions of nucleic acids. Select RNA mode (RNA-40) to measure total RNA concentration. Raise the sampling arm and pipette the 1×DPBS onto the lower measurement pedestal. Lower the sampling arm and initiate a spectral measurement using the software on the PC. Click Blank to measure and store the reference spectrum. Analyze a fresh replicate of the blank as though it were a sample by choosing Measure. The result should be a spectrum that varies no more than 0.04 A (10 mm absorbance equivalent). Raise the sampling arm and pipette the sample onto the lower measurement pedestal. Lower the sampling arm and initiate a spectral measurement using the software on the PC. When the measurement is complete, raise the sampling arm and wipe the sample from both the upper and lower pedestals using a dry, lint-free laboratory wipe. Confirm 260/280-ratio of absorbance at 260 nm and 280 nm. The ratio of absorbance at 260 and 280 nm is used to assess the purity of DNA and RNA. A ratio of ˜1.8 is generally accepted as “pure” for DNA; a ratio of ˜2.0 is generally accepted as “pure” for RNA. If the ratio is appreciably lower in either case, it may indicate the presence of protein, phenol or other contaminants that absorb strongly at or near 280 nm. Confirm 260/230-ratio of absorbance at 260 nm and 230 nm. This is a secondary measure of nucleic acid purity. The 260/230 values for a “pure” nucleic acid are often higher than the respective 260/280 values and are commonly in the range of 1.8-2.2. If the ratio is appreciably lower, this may indicate the presence of copurified contaminants. For normalization of RNA samples prior to RT-PCR, adjust sample volume from total RNA concentration. Don't exceed the total RNA amount of 1000 ng (10 uL of 100 ng per uL). Prepare duplicate tubes if positive and negative reverse transcriptase (RNA) samples are to be used in the amplification reaction. Add the following to an RNase-free, 0.5-ml microcentrifuge tube on ice. Use DNase I, Amplification Grade (Invitrogen, Cat #18-068-015); 1 uL of 10× DNase I Reaction buffer; 1 μl of DNase I, Amp Grade, 1 U per uL; 1-8 uL of the extracted total RNA sample with adjusted volume for total RNA normalization. Usually, add total RNA of 100 ng with 5 uL of 20 ng/uL; DEPC-treated RNase free water up to 10 uL. Incubate tube(s) for 15 min at room temperature. Inactivate the DNase I by the addition of 1 μl of 25 mM EDTA solution to the reaction mixture. Heat for 10 min at 65° C. The RNA sample is ready to use in reverse transcription, prior to PCR amplification. For Reverse Transcription (RT) process, use High Capacity cDNA Reverse Transcription kit with RNase Inhibitor (Applied Biosystems, Cat #43-749-66). Quantitatively converting up to 2 μg (for a 20-μL reaction) of total RNA to cDNA Prepare the 2×Reverse Transcription Master Mix: 2.0 uL of 10×RT Buffer; 0.8 uL of 25×dNTP Mix (100 mM); 2.0 uL of 10×RT Oligo (dT) Primer or Random primer; 1.0 uL of RNase Inhibitor; 1.0 uL of MultiScribe™ Reverse Transcriptase; 3.2 uL of Nuclease-free H₂O; Add DNase treated total RNA of 10 uL to the 2×RT Master Mix of 10 uL to create a 1×mix of 20 uL. Vortex briefly to mix. Centrifuge briefly to bring the reaction mix to the bottom of the tube and eliminate air bubbles. Perform reverse transcription with Oligo dT or random hexamer method in a thermal cycler for 2 hour 15 minutes. Prepare reaction mixture for qRT-PCR experiment: TaqMan® Fast Advanced Master Mix is supplied at a 2×concentration and contains: AmpliTaq™ Fast DNA Polymerase; Uracil-N glycosylase (UNG); dNTPs with dUTP; ROX™ dye (passive reference); Optimized buffer components. Keep the TaqMan® Fast Advanced Master Mix on ice. Thaw TaqMan® Assays on ice, then vortex and briefly centrifuge to resuspend. Transfer the appropriate volume of PCR reaction mix to each well of an optical reaction plate (96-well plate). 10 uL of 2×Master Mix (Applied Biosystems, TaqMan Fast Advanced Master Mix, Cat #4444557); 1 uL of 20×TaqMan assay mix with target probe and primers for TP53, VEGFA and COLIAl genes; 2 uL of cDNA template (20 ng with 2 uL of 10 ng/uL in RT sample; 7 uL of Nuclease-free H₂O. Seal the reaction plate with optical adhesive film, then centrifuge briefly to bring the PCR reaction mix to the bottom of the well and eliminate air bubbles. Open the plate document or experiment file that corresponds to the reaction plate in the system software. Load the reaction plate to the real-time PCR system. Start the run on the qPCR reaction. After all reaction is completed, view the amplification plots for the reactions. Use auto baseline and auto threshold setting to determine the threshold cycles (Ct) for the amplification curves. Use the comparative Ct (ΔC_{T}) method with GAPDH reference to analyze data. Analyze the qPCR data using ΔΔC_{T} method.

### Example 10. Enriched EV Internalization to PANC-1 Pancreatic Cancer Cells

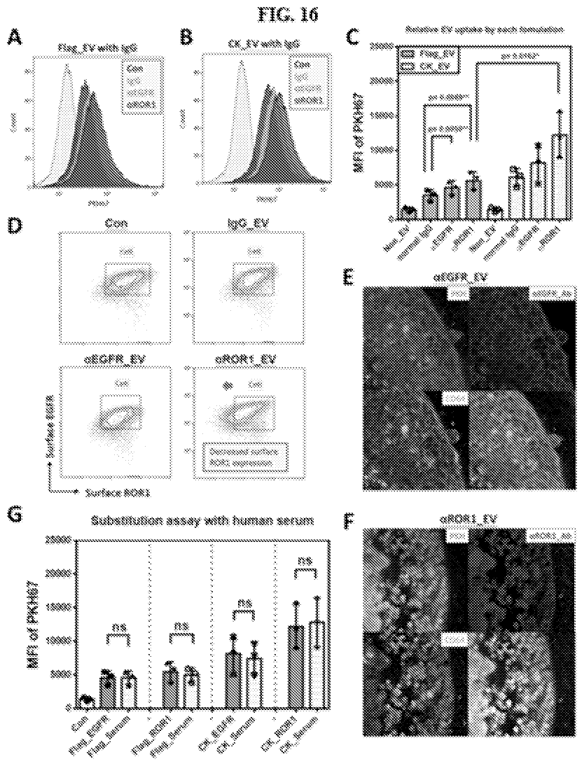

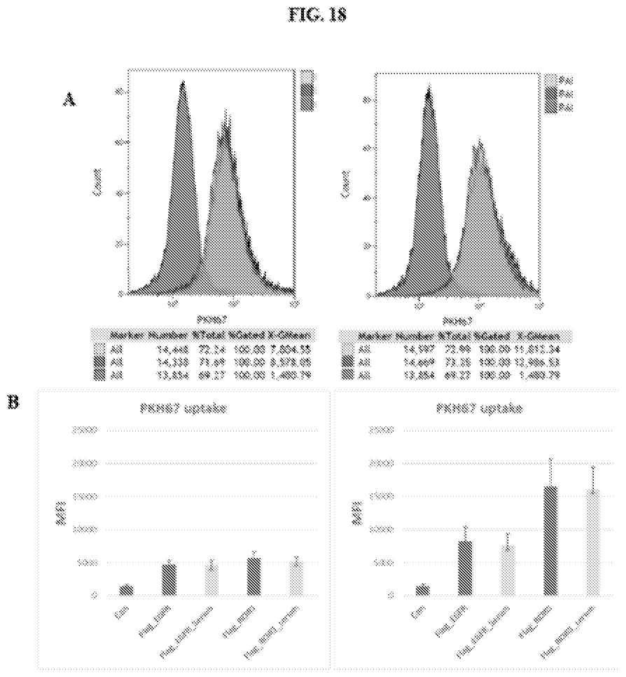

In addition to targeting, the disclosed CD64-enriched EVs comprising humanized monoclonal antibodies (hmAbs) and tissue homing peptides (THPs) on the EV surface can substantially enhance cellular internalization and tissue penetration (including transcytosis). PANC-1 cells were used as a pancreatic cancer model because of high surface expression of EGFR (Epidermal Growth Factor Receptor) and ROR1 (Receptor Tyrosine Kinase Like Orphan Receptor 1). The PANC-1 cells were cultured and incubated with CD64/EVs with one of the following targeting formulations: (i) flag-control (no targeting moiety), (ii) CK (CKAAKNK)-peptide without IgG, (iii) CK-peptide with normal IgG (IgG without specificity), (iv) CK-peptide and αhEGFR_IgG, and (v) CK-peptide and αhROR1_IgG. CD64/EVs were pre-mixed with individual hmAbs for an hour at room temperature and the unbonded hmAbs were then washed away. PANC-1 cells in monolayer culture were treated with formulated CD64/EVs with each hmAb for 24h, and then washed and suspended for flow cytometry. The flow assay was conducted to quantify the amount of internalized EVs which were fluorescence labelled with PKH67. The first comparison was the uptake efficiency of flag-(FIG. 16A) or CK-peptide (FIG. 16B) expressed EVs loaded with different hmAbs. The fluorescence intensity revealed that αhROR1-targeted-EVs were taken-up better than αhEGFR-targeted-EVs or IgG-control due to the selectivity against surface ROR1 or EGFR on PANC-1 cells. Binding the clinically available hmAbs on CD64 at the EV surface, αhROR1 in particular, could increase the amount of EVs internalized to PANC-1 cancer cells by ˜60% for αhROR and ˜30% for αhEGFR comparing to the non-targeted IgG_EVs (FIG. 16C). Moreover, additional CK-peptides on the EV surface can nearly double the uptake of αhROR1_EVs in PANC-1 cells (as shown in mean fluorescence intensity [MFI], Flag_αROR1: 5585±755.9; CK_αROR1: 112209±1914). FIG. 16C summarizes the quantitative results from the flow cytometry assay. To further quantify the hmAbs-assisted enrichment of EV uptake, the surface EGFR and ROR1 expression on PANC-1 cell was stained with or without the hmAb-targeting formulation. From the staining of PANC-1 cells with αROR1_EV treatment for 4 hours, a decrease of surface ROR1 expression was observed, implying that the αhROR1 can induce a stronger EV internalization than αhEGFR for enhanced drug delivery (FIG. 16D). An EV uptake assay on 3D tumor spheroids of PANC-1 cells was then performed. Consistently, αhROR1_EVs showed stronger surface ROR1 internalization leading to enriched EV uptake, while αhEGFR_EVs could target PANC-1 surface well but with less internalization after 6-hour incubation (FIGS. 16E and 16F). Since human IgG naturally exists in human serum at a concentration of 6-16 g/L, a substitution assay was performed to evaluate the stability of αhROR1 and αEGFR on EVs. EVs were first loaded with the targeting antibodies (i.e. αhEGFR and αhROR1) and then incubated with human serum (50%) for 6 hours at 37° C. The purpose of this substitution assay was to understand whether pre-loaded hmAbs could be replaced by serum human IgG in blood circulation. After comparing the targeting ability of EVs before and after substitution assay, no significant loss of the targeting ability was observed (FIG. 16G and FIG. 18A-B). This data supports the stability of the humanized antibody on the CD64/EVs in clinic uses.

### Example 11. Enhanced Tissue Penetration of Targeting EVs

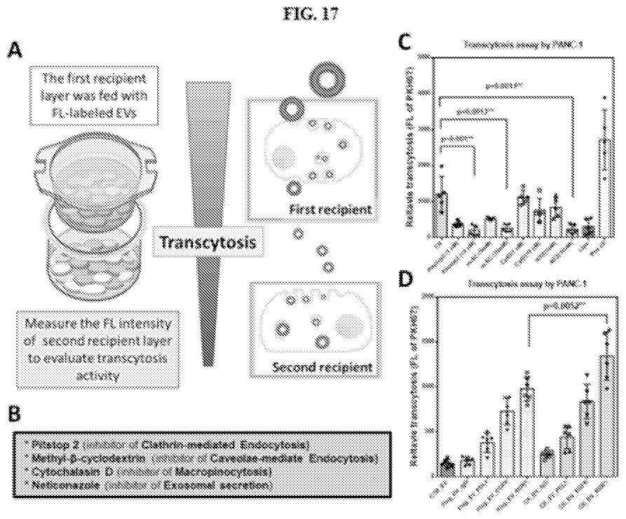

For each EV formulation with different targeting designs, a transwell-based assay was established to quantify the penetrative ability via multiple layers of PANC-1 cells. The activity of transcytosis was determined by the EV exchange between the upper and bottom layers of PANC-1 cells separated by a 5 μm pored Transwell® membrane (FIG. 17A). The upper layer was comprised of PANC-1 cells over 90% confluence in monolayer culture to mimic the tight junction of human pancreatic duct epithelial cells. The EVs were fluorescence labelled with PKH67 and incubated with upper layer cells as the first recipient. As transcytosis gradually occurred, the fluorescence labelled EVs were up taken by the first recipient cells but many would secrete into extracellular region via exocytosis. Some of the fluorescence labelled EVs in the intermediary area would later be up taken by the second recipient cells on the bottom layer, leading to detectable fluorescence signals as an indicator. A commercial PEGylated liposome, Invivofectamine (Thermofisher) was synthesized according to manufacturer instruction as a control. It was found that EVs could enter/exit the upper cell monolayer and be up taken by the bottom cell monolayer 2˜3 folds better than liposomes (FIG. 17C). Various inhibitors selected to block endocytosis and EV secretion are given in FIG. 17B. Inhibiting clathrin- and caveolae-mediated endocytosis significantly reduced the EV transcytosis, suggesting that EV entry into recipient cells (PANC-1 in this case) was mainly mediated by clathrin- and caveolae-mediated endocytosis. Interestingly, the inhibition of EV secretion of upper PANC-1 cells by neticonazole strongly decreased the transcytosis activity (FIG. 17C). With targeting hmAbs on the EV surface, the transcytosis activity could be enriched 3˜4 folds comparing to that of non-targeted EVs (FIG. 17D). Again, αhROR1-loaded CD64/EV surface could best enhance transcytosis of PANC-1 cells, and a combination of hmAb and CK-peptide on CD64/EVs would further improve transcytosis (FIG. 17D).

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A composition comprising at least one extracellular vesicle, said extracellular vesicle comprising:
a. at least one adapter polypeptide comprising a peptide sequence that binds to an Fc region of an antibody with a dissociation constant (K_{d}) of less than or equal to 10⁻⁹M, wherein said adapter polypeptide comprises an extracellular domain;
b. said antibody complexed with said adapter polypeptide, wherein said antibody binds a first cell-surface marker associated with a diseased cell; and
c. at least one therapeutic.

2. The composition of claim 1, wherein the peptide sequence that binds to an Fc region of an antibody is at least 70% identical to an Fc receptor.

3. The composition of claim 2, wherein said Fc receptor is a Fc-gamma receptor, Fc-alpha receptor, or Fc-epsilon receptor.

4. The composition of claim 2, wherein said Fc receptor comprises FcγRI (CD64), FcγRII (CD32), or FcγRIII (CD16).

5. The composition of claim 4, wherein said Fc receptor is CD64.

6. The composition of claim 1, wherein said adapter polypeptide further comprises a targeting domain that binds a second cell-surface marker associated with said diseased cell, wherein said targeting domain is attached to said extracellular domain of said adapter polypeptide.

7. The composition of claim 6, wherein said targeting domain is selected from the group consisting of a tumor homing peptide, a tumor targeting domain, a tissue-targeting domain, a cell-penetrating peptide, a viral membrane protein, and any combination or fragment thereof.

8. The composition of claim 1, wherein said diseased cell is a cancer cell or a non-cancerous lesion cell.

9. The composition of claim 1, wherein said first cell-surface marker comprises EGFR, PD-L1, or ROR1.

10. The composition of claim 6, wherein said first cell-surface marker and said second cell-surface marker are different.

11. The composition of claim 6, wherein said first cell-surface marker and said second cell-surface marker are identical.

12. The composition of claim 1, wherein said antibody is a humanized monoclonal antibody.

13. The composition of claim 1, wherein said antibody is selected from the group consisting of humanized anti-EGFR antibody clone C225, humanized anti-ROR1 antibody clone 2A2, and humanized anti-PD-L1 antibody clone SP142.

14. The composition of claim 12, wherein said humanized monoclonal antibody comprises an IgG.

15. The composition of claim 14, wherein said humanized monoclonal antibody comprises an IgG1 or IgG3.

16. The composition of claim 1, wherein said antibody is non-covalently complexed with said adapter polypeptide.

17. The composition of claim 16, wherein said Fc region of said antibody is configured to complex to said adapter polypeptide in an acidic environment.

18. The composition of claim 16, wherein said Fc region of said antibody is configured to be released from complexed to said adapter polypeptide in an acidic environment.

19. The composition of claim 1, wherein said at least one therapeutic is within said extracellular vesicle.

20. The composition of claim 1, wherein said at least one therapeutic is expressed on an extracellular surface of said extracellular vesicle.

21. The composition of claim 1, wherein said at least one therapeutic is attached to said extracellular domain.

22. The composition of claim 1, wherein said at least one therapeutic comprises a therapeutic polynucleotide, a therapeutic polypeptide, a therapeutic compound, a cancer drug, or a combination thereof.

23. The composition of claim 22, wherein said therapeutic polynucleotide comprises a messenger RNA, a microRNA, a shRNA, or a combination thereof.

24. The composition of claim 1, wherein said extracellular vesicle is an exosome, a microvesicle, or an apoptotic body.

25. The composition of claim 24, wherein said extracellular vesicle is an exosome.

26. A method of treating a subject, said method comprising administering a therapeutically effective amount of a pharmaceutical composition to said subject, wherein said pharmaceutical composition comprises said composition of any one of claims 1-25.

27. The method of claim 26, wherein said pharmaceutical composition comprises at least one pharmaceutically acceptable excipient.

28. The method of claim 26, wherein said subject has cancer or a non-cancerous lesion.

29. The method of claim 28, wherein said subject has glioma.

30. The method of claim 26, wherein said subject has muscular dystrophy.

31. The method of claim 30, wherein said muscular dystrophy is selected from the group consisting of Duchenne muscular dystrophy, Becker muscular dystrophy, facioscapulohumeral muscular dystrophy, congenital muscular dystrophy, and myotonic dystrophy.

32. The method of claim 26, wherein said subject has a retinal disease.

33. The method of claim 32, wherein said retinal disease is retinitis pigmentosa or Leber's congenital amaurosis.

34. The method of claim 26, wherein said subject is administered said therapeutically effective amount of said pharmaceutical composition at a frequency of at least once per day, once per week, once per month, or once per year.

35. The method of claim 26, wherein said pharmaceutical composition is an aqueous formulation.

36. The method of claim 26, wherein said pharmaceutical composition is formulated for injection.

37. The method of claim 26, wherein said pharmaceutical composition is administered to said subject intrathecally, intraocularly, intravitreally, retinally, intravenously, intramuscularly, intraventricularly, intracerebrally, intracerebellarly, intracerebroventricularly, intraperenchymally, subcutaneously, intranasally, or a combination thereof.

38. A method of producing a composition, said method comprising:
a. transfecting an extracellular vesicle donor cell with at least one heterologous polynucleotide encoding an adapter polypeptide, wherein said adapter polypeptide comprises a peptide sequence that is at least 70% identical to a Fc receptor, wherein said Fc receptor recognizes a Fc region of an antibody;
b. collecting an extracellular vesicle released from said extracellular vesicle donor cell, wherein said extracellular vesicle released from said extracellular vesicle donor cell expresses said adapter polypeptide, wherein said adapter polypeptide comprises an extracellular domain, and wherein said extracellular vesicle comprises at least one therapeutic; and
c. complexing said antibody to said extracellular domain, wherein said antibody binds a first cell-surface marker associated with a diseased cell.

39. The method of claim 38, wherein said Fc receptor is a Fc-gamma receptor, Fc-alpha receptor, or Fc-epsilon receptor.

40. The method of claim 39, wherein said Fc receptor is FcγRI (CD64), FcγRII (CD32), or FcγRIII (CD16).

41. The method of claim 40, wherein said Fc receptor is CD64.

42. The method of claim 38, wherein said adapter polypeptide further comprises a targeting domain that binds a second cell-surface marker associated with said diseased cell, wherein said targeting domain is attached to said extracellular domain.

43. The method of claim 42, wherein said first cell-surface marker or said second cell-surface marker is associated with a cancer cell or a non-cancerous lesion cell.

44. The method of claim 43, wherein said first cell-surface marker comprises EGFR, PD-L1, or ROR1.

45. The method of claim 43, wherein said first cell-surface marker and said second cell-surface marker are different.

46. The method of claim 43, wherein said first cell-surface marker and said second cell-surface markers are identical.

47. The method of claim 42, wherein said targeting domain is selected from the group consisting of a tumor homing peptide, a tumor targeting domain, a tissue-targeting domain, a cell-penetrating peptide, a viral membrane protein, and any combination or fragment thereof.

48. The method of claim 38, wherein said at least one therapeutic is within said extracellular vesicle.

49. The method of claim 38, wherein said at least one therapeutic is expressed on an extracellular surface of said extracellular vesicle.

50. The method of claim 38, wherein said at least one therapeutic is attached to said extracellular domain.

51. The method of claim 38, wherein said at least one therapeutic comprises a therapeutic polynucleotide, a therapeutic polypeptide, a therapeutic compound, a cancer drug, or a combination thereof.

52. The method of claim 51, wherein said therapeutic polynucleotide comprises a messenger RNA, a microRNA, a shRNA, or a combination thereof.

53. The method of claim 38, wherein said extracellular vesicle released from said extracellular vesicle donor cell is an exosome, a microvesicle, or an apoptotic body.

54. The method of claim 53, wherein said extracellular vesicle released from said extracellular vesicle donor cell is an exosome.

55. The method of claim 38, wherein transfecting said extracellular vesicle donor cell comprises electroporation, microfluidic electroporation, microchannel electroporation, or nanochannel electroporation.

56. The method of claim 55, wherein said microchannel electroporation or said nanochannel electroporation comprises use of micropore patterned silicon wafers, nanopore patterned silicon wafers, track etch membranes, ceramic micropore membranes, ceramic nanopore membranes, other porous materials, or a combination thereof.

57. The method of claim 56, wherein transfecting said extracellular vesicle donor cell comprises nanochannel electroporation, and wherein said at least one heterologous polynucleotide is nanoelectroporated into said extracellular vesicle donor cell via a nanochannel located on a biochip.

58. The method of claim 38, wherein transfecting said extracellular vesicle donor cell comprises use of a gene gun, micro-needle array, nano-needle array, sonication, or chemical permeation.

59. The method of claim 38, wherein said at least one heterologous polynucleotide is a plasmid.

60. A composition comprising at least one extracellular vesicle, comprising:
a. at least one adapter polypeptide comprising a peptide sequence that is at least 70% identical to a Fc receptor that binds to an Fc region of an antibody, wherein said adapter polypeptide comprises an extracellular domain;
b. said antibody complexed with said adapter polypeptide, wherein said antibody specifically binds a first cell-surface marker associated with an immune cell; and
c. at least one viral mimic peptide.

61. The composition of claim 60, wherein said Fc receptor is a Fc-gamma receptor, Fc-alpha receptor, or Fc-epsilon receptor.

62. The composition of claim 61, wherein said Fc receptor comprises FcγRI (CD64), FcγRII (CD32), or FcγRIII (CD16).

63. The composition of claim 62, wherein said Fc receptor is CD64.

64. The composition of claim 60, wherein said adapter polypeptide further comprises a targeting domain that binds a second cell-surface marker associated with said immune cell, wherein said targeting domain is attached to said extracellular domain of said adapter polypeptide.

65. The composition of claim 60, wherein said immune cell is a T cell, a B cell, a dendritic cell, a macrophage, or a natural killer (NK) cell.

66. The composition of claim 60, wherein said first cell-surface marker comprises LILRA4, CD3, CD19, CD20, or CD28.

67. The composition of claim 64, wherein said first cell-surface marker and said second cell-surface marker are different.

68. The composition of claim 64, wherein said first cell-surface marker and said second cell-surface marker are identical.

69. The composition of claim 60, wherein said antibody is a humanized monoclonal antibody.

70. The composition of claim 69, wherein said humanized monoclonal antibody is an IgG.

71. The composition of claim 70, wherein said IgG is an IgG1 or IgG3.

72. The composition of claim 60, wherein said antibody is non-covalently complexed with said adapter polypeptide.

73. The composition of claim 70, wherein said Fc region of said antibody is configured to complex to said adapter polypeptide in an acidic environment.

74. The composition of claim 70, wherein said Fc region of said antibody is configured to be released from complexed to said adapter polypeptide in an acidic environment.

75. The composition of claim 60, wherein said at least one viral mimic peptide is expressed on an extracellular surface of said extracellular vesicle.

76. The composition of claim 60, wherein said at least one viral mimic peptide is attached to said extracellular domain.

77. The composition of claim 60, wherein said at least one viral mimic peptide comprises a peptide sequence that is at least 70% identical with a SARS-COV-2 viral protein.

78. The composition of claim 77, wherein said SARS-COV-2 viral protein comprises an Envelopment (E) protein, a Nucleocapsid (N) protein, a Membrane (M) protein, or a Spike (S) protein.

79. The composition of claim 78, wherein said SARS-COV-2 viral protein is said S protein.

80. The composition of claim 60, wherein said extracellular vesicle comprises an exosome, a microvesicle, or an apoptotic body.

81. The composition of claim 80, wherein said extracellular vesicle is an exosome.

82. A method of vaccinating a subject, said method comprising administering a therapeutically effective amount of a pharmaceutical composition to said subject, wherein said pharmaceutical composition comprises said composition of any one of claims 60-81.

83. The method of claim 82, wherein said pharmaceutical composition comprises at least one pharmaceutically acceptable excipient.

84. The method of claim 82, wherein said subject is administered said therapeutically effective amount of said pharmaceutical composition at a frequency of at least once per day, once per week, once per month, or once per year.

85. The method of claim 82, wherein said pharmaceutical composition is an aqueous formulation.

86. The method of claim 82, wherein said pharmaceutical composition is formulated for injection.

87. The method of claim 82, wherein said pharmaceutical composition is administered to said subject intrathecally, intraocularly, intravitreally, retinally, intravenously, intramuscularly, intraventricularly, intracerebrally, intracerebellarly, intracerebroventricularly, intraperenchymally, subcutaneously, intranasally, or a combination thereof.

88. A method of producing a composition, said method comprising:
a. transfecting an extracellular vesicle donor cell with at least one heterologous polynucleotide encoding an adapter polypeptide, wherein said adapter polypeptide comprises a peptide sequence that is at least 70% identical to a Fc receptor, wherein said Fc receptor recognizes a Fc region of an antibody;
b. collecting an extracellular vesicle released from said extracellular vesicle donor cell, wherein said extracellular vesicle released from said extracellular vesicle donor cell expresses said adapter polypeptide, wherein said adapter polypeptide comprises an extracellular domain, and wherein said extracellular vesicle comprises at least one viral mimic peptide; and
c. complexing said antibody to said extracellular domain, wherein said antibody binds a first cell-surface marker associated with an immune cell.

89. The method of claim 88, wherein said Fc receptor is a Fc-gamma receptor, Fc-alpha receptor, or Fc-epsilon receptor.

90. The method of claim 89, wherein said Fc receptor comprises FcγRI (CD64), FcγRII (CD32), or FcγRIII (CD16).

91. The method of claim 90, wherein said Fc receptor is CD64.

92. The method of claim 88, wherein said adapter polypeptide further comprises a targeting domain that binds a second cell-surface marker associated with said immune cell, wherein said targeting domain is attached to said extracellular domain.

93. The method of claim 88, wherein said immune cell is a T cell, a B cell, a dendritic cell, a macrophage, or a natural killer (NK) cell.

94. The method of claim 88, wherein said first cell-surface marker comprises LILRA4, CD3, CD19, CD20, or CD28.

95. The method of claim 92, wherein said first cell-surface marker and said second cell-surface marker are different.

96. The method of claim 92, wherein said first cell-surface marker and said second cell-surface markers are identical.

97. The method of claim 92, wherein said antibody comprises a humanized monoclonal antibody.

98. The method of claim 97, wherein said antibody is an IgG.

99. The method of claim 98, wherein said IgG is an IgG1 or IgG3.

100. The method of claim 88, wherein said antibody is non-covalently complexed with said adapter polypeptide.

101. The method of claim 100, wherein said Fc region of said antibody is configured to complex to said adapter polypeptide in an acidic environment.

102. The method of claim 100, wherein said Fc region of said antibody is configured to be released from complexed to said adapter polypeptide in an acidic environment.

103. The method of claim 88, wherein said at least one viral mimic peptide is expressed on an extracellular surface of said extracellular vesicle.

104. The method of claim 88, wherein said at least one viral mimic peptide is attached to said extracellular domain.

105. The method of claim 88, wherein said at least one viral mimic peptide comprises a peptide sequence that is at least 70% identical with a SARS-COV-2 viral protein.

106. The method of claim 105, wherein said SARS-COV-2 viral protein comprises an Envelopment (E) protein, a Nucleocapsid (N) protein, a Membrane (M) protein, or a Spike (S) protein.

107. The method of claim 106, wherein said SARS-COV-2 viral protein is said S protein.

108. The method of claim 88, wherein said extracellular vesicle comprises an exosome, a microvesicle, or an apoptotic body.

109. The method of claim 108, wherein said extracellular vesicle is an exosome.

110. The method of claim 88, wherein transfecting said extracellular vesicle donor cell comprises electroporation, microfluidics electroporation, microchannel electroporation, or nanochannel electroporation.

111. The method of claim 110, wherein said microchannel electroporation or said nanochannel electroporation comprises use of micropore patterned silicon wafers, nanopore patterned silicon wafers, track etch membranes, ceramic micropore membranes, ceramic nanopore membranes, other porous materials, or a combination thereof.

112. The method of claim 111, wherein transfecting said extracellular vesicle donor cell comprises nanochannel electroporation, and wherein said at least one heterologous polynucleotide is nanoelectroporated into said extracellular vesicle donor cell via a nanochannel located on a biochip.

113. The method of claim 88, wherein transfecting said extracellular vesicle donor cell comprises a use of a gene gun, micro-needle array, nano-needle array, sonication, or chemical permeation.

114. The method of claim 88, wherein said at least one heterologous polynucleotide is a plasmid.

115. The method of claim 88, wherein the extracellular vesicle donor cell is selected from the group consisting of a mouse embryonic fibroblast (MEF), a human embryonic fibroblast (HEF), a dendritic cell, a mesenchymal stem cell, a bone marrow-derived dendritic cell, a bone marrow derived stromal cell, an adipose stromal cell, an endothelial cell, an enucleated cell, a neural stem cell, an immature dendritic cell, and an immune cell

116. The method of claim 88, wherein the extracellular vesicle donor cell is selected from the group consisting of an animal primary cell, a human primary cell, an animal cell line, and a human cell line.

117. The method of claim 88, wherein the extracellular vesicle donor cell is selected from the group consisting of a genetically modified animal primary cell, a genetically modified human primary cell, a genetically modified animal cell line, and a genetically modified human cell line.
